(19) United States
(12) Patent Application Publication
(10) Pub. No.: US 2026/0226477 A1
(43) Pub. Date: Aug. 6, 2026

(54) Transferrin Receptor (TFR) Aptamers and Use Thereof

(71) Applicant: Creyon Bio, Inc., Carlsbad, CA (US)

(72) Inventors: Matthew John Warner, Chapel Hill, NC (US); Linsley Kelly, Durham, NC (US); Matthew Levy, Chapel Hill, NC (US)

(21) Appl. No.: 19/566,865

(22) Filed: Mar. 13, 2026

Related U.S. Application Data
(63) Continuation of application No. PCT/US2025/036528, filed on Jul. 3, 2025.
(60) Provisional application No. 63/667,581, filed on Jul. 3, 2024.

(51) Int. Cl. C12N 15/115; C12N 15/113; C12N 15/88

(52) U.S. Cl. CPC C12N 15/115; C12N 15/113; C12N 15/88; C12N 2310/11; C12N 2310/14; C12N 2310/16; C12N 2310/321; C12N 2310/322; C12N 2310/3515; C12N 2310/3519

(57) ABSTRACT

The invention relates to compounds comprising advantageous novel aptamers and aptamer conjugates, as well as compositions comprising the same, and their use in therapy or diagnosis. In particular, disclosed herein are aptamers and aptamer conjugates that bind to the human transferrin receptor 1 (hTfR1) without competing with human transferrin (hTf) for binding to hTfR1.

### CROSS REFERENCE TO RELATED APPLICATIONS

This application is a continuation of PCT Application No. PCT/US2025/036528, filed Jul. 3, 2025, which application claims the benefit of U.S. Provisional Application No. 63/667,581, filed Jul. 3, 2024, all which is incorporated by reference in their entirety.

### INCORPORATION-BY-REFERENCE OF SEQUENCE LISTING

The instant application contains a sequence listing which has been submitted via Patent Center and is hereby incorporated by reference in its entirety. Said XML copy, created on Sep. 26, 2025, is named “63148WO_CRF_sequencelisting.xml”, and is 198,374 bytes in size.

### FIELD OF THE INVENTION

This invention relates to compounds comprising advantageous novel aptamers and aptamer conjugates, as well as compositions comprising the same, and their use in therapy or diagnosis. In particular, disclosed herein are aptamers and aptamer conjugates that bind to the human transferrin receptor 1 (hTfR1) without competing with human transferrin (hTf) for binding to hTfR1. In some aptamers disclosed herein, the guanosine nucleosides each comprise a 2′-fluoro-modified ribose sugar moiety, and the adenosine nucleosides, the cytosine nucleosides and the uridine nucleosides each comprise a 2′-O-methyl-modified ribose sugar moiety.

### BACKGROUND

Aptamers are folded oligonucleotide molecules that can bind to a target with high affinity and specificity. Like antibodies, this binding is mediated by the three-dimensional structure of the aptamer, which is determined by its nucleotide sequence. However, aptamers have many advantages over antibodies, including easier synthesis, lower immunogenicity, higher stability, and superior refoldability.

Human transferrin receptor 1 (hTfR1) is a cell-surface receptor. It is generally expressed at low levels, but is upregulated on cells with high-proliferation rates including activated immune cells and cancerous cells. hTfR1 is abundantly expressed on the surface of muscle cells and plays an important role in muscle development and denervation-induced muscular atrophy. hTfR1 is also expressed on brain microvascular endothelial cells and targeting hTfR1 has been shown to enhance the delivery of macromolecular and nanoparticle cargoes across the blood-brain barrier (BBB). hTfR1 is therefore a potentially important target for diagnostic techniques and delivery of therapeutics. The natural hTfR1 ligand, human transferrin (hTf), is known to be a useful targeting ligand. However, the use of hTf as a targeting ligand requires that it must compete with endogenous hTf for hTfR1 binding, and the high concentration of hTf in serum means that hTfR1 on endothelial cells is typically saturated with endogenous hTf under physiological conditions. Also, since hTf plays a central role in cellular iron metabolism, blocking hTf binding to hTfR1 may have an unwanted effect on iron homeostasis.

Previous studies have identified RNA aptamers that bind to hTfR1. Wilner et al. identified the C2 and C2.min aptamers, which share a core hTfR1 binding motif (Wilner et al., Mol Ther Nucl Acids (2012), 1, e12). C2 and C2.min are 50 and 43 nucleosides in length, respectively, and are made up of 2′-fluoro-modified pyrimidine nucleosides and unmodified (2′-hydroxyl) purine nucleosides. Maier et al. identified the aptamer Waz (Maier et al., Mol Ther Nucl Acids (2016), 5, e321). Waz is a 48-nucleotide aptamer and is also made up of 2′-fluoro-modified pyrimidine nucleosides and unmodified purine nucleosides. C2 and C2.min compete with hTf for binding to hTfR1, while Waz binds to hTfR1 outside of the hTf binding site and so does not compete with endogenous hTf. These aptamers have been developed for various biomedical applications, including targeted drug delivery and imaging. However, each of these aptamers has specific limitations that can impact their efficacy and applicability, such as low binding affinity and specificity or low stability.

Accordingly, there is a need to develop an aptamer that has improved stability, binding affinity and specificity that allow it to bind to hTfR1 but not to compete with hTf for binding to hTfR1.

### SUMMARY OF THE INVENTION

The aptamers disclosed herein bind to hTfR1 but do not compete with hTf for binding to hTfR1. In some aptamers disclosed herein, the guanosine nucleosides each comprise a 2′-fluoro-modified ribose sugar moiety, and the adenosine nucleosides, the cytosine nucleosides and the uridine nucleosides each comprise a 2′-O-methyl-modified ribose sugar moiety. Some aptamers disclosed herein benefit from surprisingly advantageous combinations of properties that enhance their suitability for their intended uses, such as their use in therapeutic or diagnostic aptamer conjugates.

Some aptamers disclosed herein are advantageous for example because they bind to hTfR1 without competing with hTf for binding to hTfR1, and because they benefit from improved in vivo stability relative to known hTfR aptamers resulting in part because all of the sugar moieties of the nucleosides in the aptamer are chemically modified. Disclosed herein are compounds comprising these advantageous aptamers. In some instances, the compound consists of an aptamer as disclosed herein.

Disclosed herein is a compound comprising an aptamer that binds to the human transferrin receptor 1 (hTfR1) and does not compete with human transferrin (hTf) for binding to hTfR1, wherein the aptamer comprises the sequence of linked nucleosides:

(SEQ ID NO: 71)
5′-XYGURGAAAKGUAAMGAUCRUUGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
Y is a nucleoside comprising a C or U nucleobase,
R is a nucleoside comprising an A or G nucleobase,
K is a nucleoside comprising a G or U nucleobase,
M is a nucleoside comprising an A or C nucleobase,
X and Z are each independently a sequence of 4 to 12 linked nucleosides comprising A, C, G or U nucleobases, and
a plurality of the underlined nucleosides interact to form a stem structure in a
computationally predicted secondary structure of the folded aptamer;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, the stem structure comprises a bulge.

In some instances, X and Z are the same length as each other. In some instances, all of the underlined nucleosides interact to form the stem structure. In some such instances, the nucleoside sequence of X is the reverse complement of the nucleoside sequence of Z.

In some instances, this compound comprises an aptamer that comprises the sequence of linked nucleosides:

(SEQ ID NO: 56)
5′-X₁UUX₂YGURGAAAKGUAAMGAUCRUUGZ₂Z₁-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
Y is a nucleoside comprising a Cor U nucleobase,
R is a nucleoside comprising an A or G nucleobase,
K is a nucleoside comprising a G or U nucleobase,
M is a nucleoside comprising an A or C nucleobase,
X₁ and Z₁ are each independently a sequence of 3 to 9 linked nucleosides comprising A, C, G or U nucleobases and X₁ and Z₁ interact with each other in a stem structure in a computationally predicted secondary structure of the folded aptamer,
X₂ and Z₂ are each independently a nucleoside comprising an A, C, G or U nucleobase and X₂ and Z₂ interact with each other in the stem structure in the computationally predicted secondary structure of the folded aptamer, and a plurality of the underlined nucleosides interact to form the stem structure in the computationally predicted secondary structure of the folded aptamer;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X₁ and Z₁ are the same length as each other. In some instances, all of the underlined nucleosides interact to form the stem structure. In some such instances, the nucleoside sequence of X₁ is the reverse complement of the nucleoside sequence of Z₁ and X₂ is complementary to Z₂.

In some instances, this compound comprises an aptamer that comprises one of the sequences of linked nucleosides of SEQ ID NO: 48, SEQ ID NO: 49, SEQ ID NO: 50, SEQ ID NO: 53 or SEQ ID NO: 54 as set forth in Table 15; SEQ ID NO: 57 or SEQ ID NO: 58 as disclosed in Example 5B; or SEQ ID NO: 64 as set forth in Table 19.

In some instances, this compound comprises an aptamer that comprises the sequence of linked nucleosides

(SEQ ID NO: 53)
5′-CGGACUUACGUAGAAAGGUAACGAUCAUUGUGUCCG-3′

wherein the nucleobases of the underlined nucleosides interact to form a stem structure in a computationally predicted secondary structure of the folded aptamer.

In some instances, the nucleobase sequence of this aptamer consists of the sequence

(SEQ ID NO: 53)
5′-CGGACUUACGUAGAAAGGUAACGAUCAUUGUGUCCG-3′

wherein the nucleobases of the underlined nucleosides interact to form a stem structure in a computationally predicted secondary structure of the folded aptamer.

In some instances, the nucleobase sequence of this aptamer consists of one the sequences of linked nucleosides of SEQ ID NO: 48, SEQ ID NO: 49, SEQ ID NO: 50, SEQ ID NO: 53 or SEQ ID NO: 54 as set forth in Table 15; SEQ ID NO: 57 or SEQ ID NO: 58 as disclosed in Example 5B; or SEQ ID NO: 64 as set forth in Table 19.

Also disclosed herein is a compound comprising an aptamer that binds to the human transferrin receptor 1 (hTfR1) and which does not compete with human transferrin (hTf) for binding to hTfR1, wherein the aptamer comprises the sequence of linked nucleosides:

(SEQ ID NO: 43)
5′-XUGURGAAAKGUAAMGAUCGUUGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
R is a nucleoside comprising an A or G nucleobase,
K is a nucleoside comprising a G or U nucleobase,
M is a nucleoside comprising an A or C nucleobase, and
X and Z are each independently a sequence of 4 or 5 linked nucleosides comprising A, C, G or U nucleobases, and
a plurality of the underlined nucleosides interact to form a stem structure in a computationally predicted secondary structure of the folded aptamer;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X and Z are the same length as each other. In some instances, all of the underlined nucleosides interact to form the stem structure. In some such instances, the nucleoside sequence of X is the reverse complement of the nucleoside sequence of Z.

In some instances, this compound comprises an aptamer that comprises one of the sequences of linked nucleosides of SEQ ID NO: 26, SEQ ID NO: 27, or SEQ ID NO: 37 as set forth in Table 12; SEQ ID NO: 27, SEQ ID NO: 44, or SEQ ID NO: 45 as set forth in Table 13; SEQ ID NO: 37, SEQ ID NO: 45, or SEQ ID NO: 52 as set forth in Table 15.

In some instances, this compound comprises an aptamer that comprises the sequence of linked nucleosides

(SEQ ID NO: 45)
5′-CGCGGUGUGGAAAUGUAAAGAUCGUUGCCGCG-3′

wherein the nucleobases of the underlined nucleosides interact to form a stem structure in a computationally predicted secondary structure of the folded aptamer.

In some instances, the nucleobase sequence of this aptamer consists of the sequence

(SEQ ID NO: 45)
5′-CGCGGUGUGGAAAUGUAAAGAUCGUUGCCGCG-3′

wherein the nucleobases of the underlined nucleosides interact to form a stem structure in a computationally predicted secondary structure of the folded aptamer.

In some instances, the nucleobase sequence of this aptamer consists of one of the sequences of linked nucleosides of SEQ ID NO: 26, SEQ ID NO: 27, or SEQ ID NO: 37 as set forth in Table 12; SEQ ID NO: 27, SEQ ID NO: 44, or SEQ ID NO: 45 as set forth in Table 13; SEQ ID NO: 37, SEQ ID NO: 45, or SEQ ID NO: 52 as set forth in Table 15.

Also disclosed herein is a compound that comprises an aptamer that binds to the human transferrin receptor 1 (hTfR1) and which does not compete with human transferrin (hTf) for binding to hTfR1, wherein the aptamer comprises the sequence of linked nucleosides:

(SEQ ID NO: 38)
5′-XCCCGGWGGGAUAGGAOUUAUCGGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
W is a nucleoside comprising an A or U nucleobase,
O is a nucleoside comprising an A nucleobase or an absent nucleoside, and
X and Z are each independently a sequence of 2 to 5 linked nucleosides comprising A, C, G or U nucleobases, and
a plurality of the underlined nucleosides interact to form a stem structure in a computationally predicted secondary structure of the folded aptamer;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X and Z are the same length as each other. In some instances, all of the underlined nucleosides interact to form the stem structure. In some such instances, the nucleoside sequence of X is the reverse complement of the nucleoside sequence of Z.

In some instances, this compound comprises an aptamer that comprises one of the sequences of linked nucleosides of SEQ ID NO: 15, SEQ ID NO: 16, or SEQ ID NO: 17, SEQ ID NO: 19 as set forth in Table 10; or SEQ ID NO: 15, SEQ ID NO: 39, or SEQ ID NO: 40, or SEQ ID NO: 41 as set forth in Table 11.

In some instances, this compound comprises an aptamer that comprises the sequence of linked nucleosides

(SEQ ID NO: 41)
5′-GCAACCCGGAGGGAUAGGAAUUAUCGGUUGC-3′

wherein the nucleobases of the underlined nucleosides interact to form a stem structure in a computationally predicted secondary structure of the folded aptamer.

In some instances, the nucleobase sequence of this aptamer consists of the sequence

(SEQ ID NO: 41)
5′-GCAACCCGGAGGGAUAGGAAUUAUCGGUUGC-3′

wherein the nucleobases of the underlined nucleosides interact to form a stem structure in a computationally predicted secondary structure of the folded aptamer.

In some instances, the nucleobase sequence of this aptamer consists of one of the sequences of linked nucleosides of SEQ ID NO: 15, SEQ ID NO: 16, or SEQ ID NO: 17, SEQ ID NO: 19 as set forth in Table 10; or SEQ ID NO: 15, SEQ ID NO: 39, or SEQ ID NO: 40, or SEQ ID NO: 41 as set forth in Table 11. In some instances, any of the aptamers disclosed herein is 25-50 nucleosides in length, 26-44 nucleosides in length, 25-40 nucleosides in length, or 30-37 nucleosides in length.

In some instances, any of the aptamers disclosed herein is less than 40 nucleosides in length or less than 37 nucleosides in length.

In some instances, any of the aptamers disclosed herein may be generated with a 3′ inverted dT modification. In some instances, any of the aptamers disclosed herein may be generated with a 5′ Thiol-Modifier C₆S—S(1-O-Dimethoxytrityl-hexyl-disulfide, 1′-[(2-cyanoethyl)-(N,N-diisopropyl)]-phosphoramidite).

Preferably, the aptamer binds to hTfR1 on the surface of human cells and the compound is subsequently internalized by the human cells.

Some of the particularly advantageous compounds disclosed herein comprise an aptamer that (i) binds to hTfR1 on the surface of human cells and the compound is subsequently internalized by the human cells; and also (ii) binds to transferrin receptor 1 (TfR1) on the surface of cells of a non-human primate (NHP) and the compound is subsequently internalized by the NHP cells. In some of these instances, the aptamer does not compete with Tf of the NHP for binding to the NHP TfR1.

The cells expressing hTfR1 or TfR1 on their surface may be brain capillary endothelial cells (BCECs) of the type that form the blood-brain barrier (BBB).

The cells expressing hTfR1 or TfR1 on their surface may be muscle cells.

The cells expressing hTfR1 or TfR1 on their surface may be skeletal muscle cells.

The cells expressing hTfR1 or TfR1 on their surface may be cardiac muscle cells.

The cells expressing hTfR1 or TfR1 on their surface may be cancer cells.

In any of these instances of aptamer binding and internalization by NHP cells, the NHP may be cynomolgus monkey.

Also disclosed herein is a compound that is an aptamer conjugate in which an aptamer as disclosed herein is conjugated to one or more of a detectable label, an antisense compound, a small organic molecule of less than 2000 Daltons (Da), polyethylene glycol or a nanoparticle. In some instances, the compound consists of an aptamer conjugate as disclosed herein.

In the aptamer conjugate, the aptamer may be conjugated to a detectable label. The detectable label may be a fluorescent dye molecule, such as a fluorescent dye molecule selected from Alexa Fluor 488 (AF488), Fluorescein, Cy3, Cy5, Alexa Fluor 647 (AF647), DyLight 650 (DY650), BP Fluor 647 (BP647), Alexa Fluor 750 (AF750) and IRDye® 800CW. The detectable label may be conjugated to the 5′ end of the aptamer via a 5′ amide linkage or a 5′ thioester linkage.

In the aptamer conjugate, the aptamer may be conjugated to an antisense compound. The antisense compound conjugated to the aptamer may be, for example, a single-stranded antisense oligonucleotide or an siRNA.

In the aptamer conjugate, the aptamer may be conjugated to a nanoparticle. In some instances, the nanoparticle is a lipid nanoparticle (LNP). In some of these instances, the LNP comprises an ionizable cationic lipid, a helper lipid, sterol lipid and a PEG-modified lipid. The LNP may comprise SM-102, DSPC, cholesterol and DMG-PEG2000. The LNP may comprise an aptamer-lipid conjugate. The aptamer-lipid conjugate may be an aptamer-DSPE conjugate. The LNP may contain a nucleic acid, and in these instances the nucleic acid may for example be a single-stranded antisense oligonucleotide, an siRNA or an mRNA.

In the aptamer conjugate, the aptamer may be conjugated to PEG.

Also disclosed herein is a composition comprising any of the compounds disclosed herein and one or more carriers or diluents.

Also disclosed herein is a composition comprising a plurality of different compounds as disclosed herein and one or more carriers or diluents.

Also disclosed herein is a pharmaceutical composition comprising any of the compounds disclosed herein and one or more pharmaceutically acceptable carriers or diluents.

Also disclosed herein is a pharmaceutical composition comprising a plurality of different compounds as disclosed herein and one or more pharmaceutically acceptable carriers or diluents.

Also disclosed herein is a pharmaceutical composition comprising any of the compounds disclosed herein and one or more pharmaceutically acceptable carriers or diluents, for use in treating a disease or condition in a mammalian subject in need thereof, the use comprising administering a therapeutically effective amount of the pharmaceutical composition to the subject in need thereof and thereby treating the disease or condition.

Also disclosed herein is the use of a pharmaceutical composition comprising any of the compounds disclosed herein and one or more pharmaceutically acceptable carriers or diluents in the manufacture of a medicament for use in treating a disease or condition in a mammalian subject in need thereof.

In these uses, the disease or condition may be a disease or condition of the brain. In such uses, following administration of the composition to the subject, the compound may cross the blood-brain barrier (BBB) from the blood into the brain. In such uses, the composition may be administered using a systemic administration route.

In these uses, the disease or condition may be a disease or condition of the muscle. In these uses, the disease or condition may be a disease or condition of the skeletal muscle. In these uses, the disease or condition may be a disease or condition of the cardiac muscle. In such uses, following administration of the composition to the subject, the compound may be internalized by the muscle cells.

In these uses, the disease or condition may be a cancer. In such uses, the disease or condition may be prostate cancer or cervical cancer. In such uses, following administration of the composition to the subject, the compound may be internalized by the cancer cells.

Also disclosed herein is a method of treating a disease or condition in a mammalian subject in need thereof, the method comprising administering a therapeutically effective amount of a pharmaceutical composition comprising any of the compounds disclosed herein and one or more pharmaceutically acceptable carriers or diluents to the subject in need thereof and thereby treating the disease or condition.

In these methods, the disease or condition may be a disease or condition of the brain. In such methods, following administration of the composition to the subject, the compound may cross the blood-brain barrier (BBB) from the blood into the brain. In such uses, the composition may be administered using a systemic administration route.

In these methods, the disease or condition may be a disease or condition of the muscle. In these methods, the disease or condition may be a disease or condition of the skeletal muscle. In these methods, the disease or condition may be a disease or condition of the cardiac muscle. In such methods, following administration of the composition to the subject, the aptamer or aptamer conjugate may be internalized by the muscle cells.

In these methods, the disease or condition may be a cancer. In such methods, the disease or condition may be prostate cancer or cervical cancer. In such methods, following administration of the composition to the subject, the compound may be internalized by the cancer cells.

Also disclosed herein is a method of a delivering a therapeutic or diagnostic agent to the brain of a mammalian subject, the method comprising administering any of the compounds that comprise aptamer conjugates as disclosed herein directly or indirectly to the bloodstream of the mammalian subject, wherein following administration of the compound to the mammalian subject the aptamer conjugate binds to the transferrin receptor on the surface of brain capillary endothelial cells (BCECs) forming the blood-brain barrier (BBB) and crosses the BBB from the bloodstream into the brain of the mammalian subject. In such methods, the compound may be administered directly to the bloodstream of the mammalian subject via intravenous administration. In such methods, the compound may be administered indirectly to the bloodstream of the mammalian subject via subcutaneous injection.

Also disclosed herein is an in vitro method of delivering a therapeutic or diagnostic agent into mammalian cells expressing a transferrin receptor on their surface, the method comprising contacting the mammalian cells with any of the compounds that comprise aptamer conjugates as disclosed herein, wherein the aptamer conjugate binds to the transferrin receptor on the surface of the mammalian cells and the compound is subsequently internalized by the mammalian cells.

In some such in vitro methods, the mammalian cells may be human or non-human primate muscle cells, such as skeletal muscle cells or cardiac muscle cells. In such in vitro methods, the muscle cells are human muscle cells or cynomolgus monkey muscle cells.

In some such in vitro methods, the mammalian cells are human cancer cells that overexpress the human transferrin receptor on their surface. In such in vitro methods, the cells may be cervical cancer cells (such as HeLa cells) or maybe prostate cancer cells (such as 22Rv1 or PC3 prostate cancer cells).

Also disclosed herein is a method of delivering a therapeutic or diagnostic agent into target cells of a mammalian subject, wherein the target cells of the mammalian subject express a mammalian transferrin receptor on their surface, the method comprising administering any of the compounds that comprise aptamer conjugates as disclosed herein to the mammalian subject, wherein following administration of the compound to the mammalian subject the aptamer binds to the transferrin receptor on the surface of the target mammalian cells and the compound is subsequently internalized by the target mammalian cells.

In any of these uses and methods involving a mammalian subject, the mammalian subject may be a primate, such as a non-human primate (for example, a cynomolgus monkey) or a human. In the context of the present invention, those uses and methods in which the mammalian subject is a cynomolgus monkey or a human are especially preferred, particularly those uses and methods in which the mammalian subject is a human.

In the disclosed method of delivering a therapeutic or diagnostic agent into target cells of a mammalian subject, the target cells may be human or non-human primate muscle cells. In particular, the muscle cells may be human or non-human primate skeletal muscle cells or may be human or non-human primate cardiac muscle cells. In some such methods, the muscle cells are human muscle cells.

In the disclosed method of delivering a therapeutic or diagnostic agent into target cells of a mammalian subject, the target cells may be human cancer cells that overexpress hTfR1 on their surface. For example, the target cells may be cervical cancer cells or prostate cancer cells.

The disclosed methods may involve the use of a compound comprising a therapeutic or diagnostic agent conjugated to an aptamer. In these uses, the agent may be a therapeutic agent, for example an antisense compound (such as a single-stranded antisense oligonucleotide or an siRNA), a small organic molecule of less than 2000 Daltons (Da), or a nanoparticle (e.g., a lipid nanoparticle).

Other features of the present invention will become apparent from the following detailed description. It should be understood, however, that the detailed description and the specific examples, while indicating particular features and instances of the disclosed invention, are given by way of illustration only. Various changes and modifications within the spirit and scope of the disclosure will become apparent to those skilled in the art from this detailed description.

### DETAILED DESCRIPTION

### Aptamers

Compounds comprising aptamers are disclosed herein. An “aptamer” herein is an oligonucleotide sequence folded into a three-dimensional structure and capable of binding to a target molecule. In some instances, the compound comprises any one of the aptamers disclosed herein. In some instances, the compound consists of any one of the aptamers disclosed herein.

Specifically, the present disclosure provides an aptamer that binds to hTfR1 without competing with hTf for binding to hTfR1. In some embodiments, the aptamer comprises:
a core having a sequence selected from:

(SEQ ID NO: 100)
5′-YGURGAAAKGUAAMGAUCRUUG-3′,
(SEQ ID NO: 101)
5′-UGURGAAAKGUAAMGAUCGUUG-3′,
or
(SEQ ID NO: 102)
5′-CCCGGWGGGAUAGGAOUUAUCGG-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
Y is a nucleoside comprising a C or U nucleobase,
R is a nucleoside comprising an A or G nucleobase,
K is a nucleoside comprising a G or U nucleobase,
M is a nucleoside comprising an A or C nucleobase,
W is a nucleoside comprising an A or U nucleobase, and
O is a nucleoside comprising an A nucleobase or an absent nucleoside;
a 5′ stem region (X) linked to 5′-end of the core and comprising nucleosides comprising A, C, G or U nucleobases; and
a 3′ stem region (Z) linked to 3′-end of the core and comprising nucleosides comprising A, C, G or U nucleobases,
wherein the 5′ stem region, the 3′ stem region and optionally, one or more nucleosides in the core can interact to form a stem structure. In some embodiments, the 5′ stem region and the 3′ stem region comprise complementary sequences.

In some embodiments, the stem structure comprises one or more bulges. In some embodiments, the stem structure comprises one bulge. In some embodiments, the bulge comprises 1 to 10 nucleosides. In some embodiments, the bulge comprises 2, 3, 5, 6, 7, or 8 nucleosides. In some embodiments, the bulge comprises 2 U (uracil) nucleobases.

In some embodiments, the aptamer comprises one or more nucleosides comprising a modified sugar moiety for increasing stability of the aptamer. In some embodiments, all the nucleosides in the aptamer comprises a modified sugar moiety for increasing stability.

In some embodiments, the aptamer comprises one or more nucleosides with phosphorothioate modification. In some embodiments, all the nucleosides in the aptamer have phosphorothioate modification.

In some embodiments, the aptamer comprises one or more guanosine nucleosides comprising a 2′-fluoro-modified ribose sugar moiety. In some embodiments, the aptamer comprises one or more cytosine nucleosides comprising a 2′-fluoro-modified ribose sugar moiety. In some embodiments, the aptamer comprises one or more adenosine nucleosides comprising a 2′-fluoro-modified ribose sugar moiety. In some embodiments, the aptamer comprises one or more uridine nucleosides comprising a 2′-fluoro-modified ribose sugar moiety.

In some embodiments, the aptamer comprises one or more guanosine nucleosides comprising a 2′-O-methyl-modified ribose sugar moiety. In some embodiments, the aptamer comprises one or more adenosine nucleosides comprising a 2′-O-methyl-modified ribose sugar moiety. In some embodiments, the aptamer comprises one or more cytosine nucleosides comprising a 2′-O-methyl-modified ribose sugar moiety. In some embodiments, the aptamer comprises one or more uridine nucleosides comprising a 2′-O-methyl-modified ribose sugar moiety.

In some embodiments, the aptamer comprises additional modification. In some embodiments, the aptamer comprises thiol modification. In some embodiments, the aptamer comprises a 5′ C6 terminal thiol (5SS).

In some embodiments, the aptamer comprises one or more guanosine nucleosides comprising a 2′-fluoro-modified ribose sugar moiety, and one or more adenosine, cytosine and uridine nucleosides comprising a 2′-O-methyl-modified ribose sugar moiety.

In some embodiments, each and every guanosine nucleoside in the aptamer comprises a 2′-fluoro-modified ribose sugar moiety. In some embodiments, each and every adenosine, cytosine and uridine nucleoside in the aptamer comprises a 2′-O-methyl-modified ribose sugar moiety.

In some embodiments, 5′ stem region (X) comprises 1 to 20 linked nucleosides. In some embodiments, 5′ stem region (X) comprises 2 to 20 linked nucleosides. In some embodiments, 5′ stem region (X) comprises 4 to 20 linked nucleosides. In some embodiments, 5′ stem region (X) comprises 4 to 18 linked nucleosides. In some embodiments, 5′ stem region (X) comprises 4 to 16 linked nucleosides. In some embodiments, 5′ stem region (X) comprises 4 to 12 linked nucleosides. In some embodiments, 5′ stem region (X) comprises 4 to 10 linked nucleosides. In some embodiments, 5′ stem region (X) comprises 6 to 12 linked nucleosides. In some embodiments, 5′ stem region (X) comprises 8 to 12 linked nucleosides.

In some embodiments, 3′ stem region (Z) comprises 1 to 20 linked nucleosides. In some embodiments, 3′ stem region (Z) comprises 4 to 20 linked nucleosides. In some embodiments, 3′ stem region (Z) comprises 4 to 18 linked nucleosides. In some embodiments, 3′ stem region (Z) comprises 4 to 16 linked nucleosides. In some embodiments, 3′ stem region (Z) comprises 4 to 14 linked nucleosides. In some embodiments, 3′ stem region (Z) comprises 4 to 12 linked nucleosides. In some embodiments, 3′ stem region (Z) comprises 4 to 10 linked nucleosides. In some embodiments, 3′ stem region (Z) comprises 6 to 12 linked nucleosides. In some embodiments, 3′ stem region (Z) comprises 8 to 12 linked nucleosides.

In some embodiments, the aptamer comprises a core having a core sequence from one aptamer selected from SEQ ID NO: 71, 53, 56, 48, 49, 50, 54, 57, 58, 43, 45, 26, 27, 37, 44, 52, 38, 41, 15, 16, 17, 19, 39, and 40. In some embodiments, the aptamer comprises a core having at least 98%, 99% or 100% sequence identity to SEQ ID NO: 105, 108, 111, 114, 117, 120, 123, 126, 129, 132, 135, 138, 141, 144, 147, 150, 153, 156, 159, 162, or 165.

In some embodiments, the aptamer comprises a 5′ stem region (X) having a 5′ stem region sequence from one aptamer selected from SEQ ID NO: 71, 53, 56, 48, 49, 50, 54, 57, 58, 43, 45, 26, 27, 37, 44, 52, 38, 41, 15, 16, 17, 19, 39, and 40. In some embodiments, the aptamer comprises a 3′ stem region (Z) having a 3′ stem region sequence from one aptamer selected from SEQ ID NO: 71, 53, 56, 48, 49, 50, 54, 57, 58, 43, 45, 26, 27, 37, 44, 52, 38, 41, 15, 16, 17, 19, 39, and 40. In some embodiments, the aptamer comprises a 5′ stem region (X) and a 3′ stem region (Z) pair having a 5′ stem region (X) and a 3′ stem region (Z) sequence from one aptamer selected from SEQ ID NO: 71, 53, 56, 48, 49, 50, 54, 57, 58, 43, 45, 26, 27, 37, 44, 52, 38, 41, 15, 16, 17, 19, 39, and 40.

In some embodiments, the aptamer comprises a core having a core sequence from one of the aptamer selected from AP-00385, AP-00382, AP-00388, AP-00389, AP-00414, AP-00264, AP-00352, AP-00257, AP-00258, AP-00259, AP-00260, AP-00353, AP-00261 AP-00392, AP-00341, AP-00248; AP-00030, AP-00039; AP-00242, AP-00031, AP-00040; AP-00243, AP-00032, AP-00041; AP-00244, AP-00034, AP-00043; AP-00245, AP-00246, and AP-00247.

In some embodiments, the aptamer comprises a 5′ stem region (X) having a 5′ stem region sequence from one aptamer selected from AP-00385, AP-00382, AP-00388, AP-00389, AP-00414, AP-00264, AP-00352, AP-00257, AP-00258, AP-00259, AP-00260, AP-00353, AP-00261 AP-00392, AP-00341, AP-00248; AP-00030, AP-00039; AP-00242, AP-00031, AP-00040; AP-00243, AP-00032, AP-00041; AP-00244, AP-00034, AP-00043; AP-00245, AP-00246, and AP-00247.

In some embodiments, the aptamer comprises a 3′ stem region (Z) having a 3′ stem region sequence from one aptamer selected from AP-00385, AP-00382, AP-00388, AP-00389, AP-00414, AP-00264, AP-00352, AP-00257, AP-00258, AP-00259, AP-00260, AP-00353, AP-00261 AP-00392, AP-00341, AP-00248; AP-00030, AP-00039; AP-00242, AP-00031, AP-00040; AP-00243, AP-00032, AP-00041; AP-00244, AP-00034, AP-00043; AP-00245, AP-00246, and AP-00247.

In some embodiments, the aptamer comprises a 5′ stem region (X) and a 3′ stem region (Z) pair from one aptamer selected from AP-00385, AP-00382, AP-00388, AP-00389, AP-00414, AP-00264, AP-00352, AP-00257, AP-00258, AP-00259, AP-00260, AP-00353, AP-00261 AP-00392, AP-00341, AP-00248; AP-00030, AP-00039; AP-00242, AP-00031, AP-00040; AP-00243, AP-00032, AP-00041; AP-00244, AP-00034, AP-00043; AP-00245, AP-00246, and AP-00247.

In some embodiments, the aptamer comprises a 5′-stem region (X) and 3′-stem region (Z) pair selected from
a. SEQ ID NO: 104 and SEQ ID NO: 106;
b. SEQ ID NO: 107 and SEQ ID NO: 109;
c. SEQ ID NO: 110 and SEQ ID NO:112;
d. SEQ ID NO:113 and SEQ ID NO: 115;
e. SEQ ID NO: 116 and SEQ ID NO: 118;
f. SEQ ID NO:119 and SEQ ID NO: 121;
g. SEQ ID NO: 122 and SEQ ID NO:124;
h. SEQ ID NO: 125 and SEQ ID NO: 127;
i. SEQ ID NO:128 and SEQ ID NO: 130;
j. SEQ ID NO: 131 and SEQ ID NO: 133;
k. SEQ ID NO: 134 and SEQ ID NO: 136;
l. SEQ ID NO: 137 and SEQ ID NO: 139;
m. SEQ ID NO: 140 and SEQ ID NO: 142;
n. SEQ ID NO: 143 and SEQ ID NO: 145;
o. SEQ ID NO: 146 and SEQ ID NO: 148;
p. SEQ ID NO: 149 and SEQ ID NO: 151;
q. SEQ ID NO: 152 and SEQ ID NO: 154;
r. SEQ ID NO: 155 and SEQ ID NO: 157;
S. SEQ ID NO: 158 and SEQ ID NO: 160;
t. SEQ ID NO: 161 and SEQ ID NO: 163; and
SEQ ID NO: 165 and SEQ ID NO: 166.

In some embodiments, the aptamer comprises a sequence selected from SEQ ID NO: 71, 53, 56, 48, 49, 50, 54, 57, 58, 43, 45, 26, 27, 37, 44, 52, 38, 41, 15, 16, 17, 19, 39, and 40.

In some embodiments, the aptamer has a sequence selected from SEQ ID NO: 71, 53, 56, 48, 49, 50, 54, 57, 58, 43, 45, 26, 27, 37, 44, 52, 38, 41, 15, 16, 17, 19, 39, and 40.

In some embodiments, the aptamer comprises a sequence having at least 90%, 95%, 98% or 99% identity to a sequence selected from SEQ ID NO: 71, 53, 56, 48, 49, 50, 54, 57, 58, 43, 45, 26, 27, 37, 44, 52, 38, 41, 15, 16, 17, 19, 39, and 40.

In some embodiments, the aptamer comprises a core having at least 90%, 95%, 98% or 99% identity to the core of one aptamer selected from AP-00385, AP-00382, AP-00388, AP-00389, AP-00414, AP-00264, AP-00352, AP-00257, AP-00258, AP-00259, AP-00260, AP-00353, AP-00261 AP-00392, AP-00341, AP-00248; AP-00030, AP-00039; AP-00242, AP-00031, AP-00040; AP-00243, AP-00032, AP-00041; AP-00244, AP-00034, AP-00043; AP-00245, AP-00246, and AP-00247.

In some embodiments, the aptamer is between 30 and 60 nucleosides in length. In some embodiments, the aptamer is between 25 and 40 nucleosides in length. In some embodiments, the aptamer is between 25 and 50 nucleosides in length. In some embodiments, the aptamer is between 25 and 40 nucleosides in length. In some embodiments, the aptamer is shorter than 45 nucleosides in length. In some embodiments, the aptamer is shorter than 40 nucleosides in length. In some embodiments, the aptamer is shorter than 35 nucleosides in length. In some embodiments, the aptamer is shorter than 30 nucleosides in length. In some embodiments, the aptamer is shorter than 25 nucleosides in length. In some embodiments, the aptamer is shorter than 20 nucleosides in length. In some embodiments, the aptamer is 30-37 nucleosides in length, or 22-44 nucleotides in length, or 31-36 nucleotides in length, or 31 nucleotides in length, or 32 nucleotides in length, or 36 nucleotides in length.

In some embodiments, the aptamer is selected from the group consisting of: AP-00385, AP-00382, AP-00388, AP-00389, AP-00414, AP-00264, AP-00352, AP-00257, AP-00258, AP-00259, AP-00260, AP-00353, AP-00261 AP-00392, AP-00341, AP-00248; AP-00030, AP-00039; AP-00242, AP-00031, AP-00040; AP-00243, AP-00032, AP-00041; AP-00244, AP-00034, AP-00043; AP-00245, AP-00246, and AP-00247.

In some embodiments, the aptamer is any one of the aptamers disclosed in Examples 1-17. In some embodiments, the aptamer is any one of the aptamers disclosed in Table A.

In some embodiments, one or more nucleosides in the 5′ stem region and one more nucleoside in the 3′ stem region interact to form a stem structure. In some embodiments, one or more nucleosides in the 5′ stem region, one more nucleoside in the 3′ stem region and one or more nucleosides in the core interact to form a stem structure. In some embodiments, one or more nucleosides in the 5′ stem region, one more nucleoside in the 3′ stem region, one nucleoside at the 5′ terminus of the core or one nucleoside at the 3′ terminus of the core interact to form a stem structure. In some embodiments, one or more nucleosides in the 5′ stem region, one more nucleoside in the 3′ stem region, one nucleoside at the 5′ terminus of the core and one nucleoside at the 3′ terminus of the core interact to form a stem structure.

In some embodiments, the aptamer has an unfolded structure. In some embodiments, the aptamer has a folded structure.

In some embodiments, the 5′-stem region comprises a repeat of the same nucleosides that can form a bulge. In some embodiments, the 5′stem region comprises 2 to 8 uracil (U(2-8)) repeat that can form a bulge. In some embodiments, the 5′stem region comprises 2 to 6 uracil (U(2-6)) repeat that can form a bulge. In some embodiments, the 5′stem region comprises 2 to 4 uracil (U(2-4)) repeat that can form a bulge. In some embodiments, the 5′stem region comprises 2 uracil (UU) repeat that can form a bulge.

In some embodiments, a plurality of the underlined nucleosides in Table A can interact to form a stem structure. In some embodiments the stem structure comprises one or more bulges.

Table A shows exemplary 5′-stem region, core, and 3′-stem region sequences for given aptamers.

TABLE A

| | Aptamer | 5′-stem region | | 3′-stem region |
|---|---|---|---|---|
| | sequence* | (X) | Core sequence | (Z) |
| | SEQ ID NO: | X | YGURGAAAKGUAAMGAUCR | Z |
| | 71 | | UUG(SEQ ID NO: 100) | |
| AP-00385 | SEQ ID NO: | CGGACUUA | CGUAGAAAGGUAACGAUCA | UGUCCG (SEQ |
| | 53 | (SEQ ID NO: 104) | UUG (SEQ ID NO: 105) | ID NO: 106) |
| | SEQ ID NO: | X₁UUX₂ | YGURGAAAKGUAAMGAUCR | Z₂Z₁ |
| | 56 | (SEQ ID NO: 107) | UUG (SEQ ID NO: 108) | (SEQ ID NO: 109) |
| AP-00382 | SEQ ID NO: | CGGAGACUCG | AGUAGAAAGGUAACGAUCA | UGUCGUAUCG |
| | 48 | GUAGACUUC | UUG | AUUCG (SEQ ID |
| | | (SEQ ID NO: 110) | (SEQ ID NO: 111) | NO: 112) |
| AP-00388 | SEQ ID NO: | GGCAGAGUCG | CGUAGAAAGGUAACGAUCA | UGCUCACUGU |
| | 49 | GUGGGCUUA | UUG (SEQ ID NO: 114) | CAUGU (SEQ ID |
| | | (SEQ ID NO: 113) | | NO: 115) |
| AP-00389 | SEQ ID NO: | UGAAAAAUUA | GUAGAAAGGUAACGAUCAU | UGCUGACUGA |
| | 50 | GUCGGCUUAC | UG (SEQ ID NO: 117) | AGUCU (SEQ ID |
| | | (SEQ ID NO: 116) | | NO: 118) |
| AP-00414 | SEQ ID NO: | cgcGACUUA | CGUAGAAAGGUAACGAUCA | UGUCgcg (SEQ |
| | 54 | (SEQ ID NO: 119) | UUG (SEQ ID NO: 120) | ID NO: 121) |
| | SEQ ID NO: | GACUUA (SEQ | CGUAGAAAGGUAACGAUCA | UGUC_(SEQ ID |
| | 57 | ID NO: 122) | UUG (SEQ ID NO: 123) | NO: 124) |
| | SEQ ID NO:58 | S(2-3)GACUUA | CGUAGAAAGGUAACGAUCA | GUCS′(2-3) (SEQ |
| | | (SEQ ID NO: 125) | UUG (SEQ ID NO: 126) | ID NO: 127) |
| | SEQ ID NO: | X | UGURGAAAKGUAAMGAUCG | Z |
| | 43 | | UUG (SEQ ID NO: 101) | |
| AP-00264 | SEQ ID NO: | CGCGG (SEQ ID | UGUGGAAAUGUAAAGAUCG | CCGCG (SEQ ID |
| | 45 | NO: 128) | UUG (SEQ ID NO: 129) | NO: 130) |
| AP- | SEQ ID NO: | CGCGG (SEQ ID | UGUGGAAAUGUAAAGAUCG | CCGCGAACGA |
| 00257; | 26 | NO: 131) | UUG (SEQ ID NO: 132) | CGGUUGUUUU |
| AP-00258 | | | | (SEQ ID NO: 133) |
| AP- | SEQ ID NO: | CGCGG (SEQ ID | UGUGGAAAUGUAAAGAUCG | CCGCGAACGA |
| 00259; | 27 | NO: 134) | UUG (SEQ ID NO: 135) | CGGUUGUUUU |
| AP-00260 | | | | UU (SEQ ID NO: |
| | | | | 136) |
| AP-00353 | SEQ ID NO: | GGGAGAGUCG | UGUAGAAAGGUAACGAUCG | GACUCACUGU |
| | 37 | GUAGCAGUC | UUG (SEQ ID NO: 138) | AAUCU (SEQ ID |
| | | (SEQ ID NO: | | NO: 139) |
| | | 137) | | |
| AP-00261 | SEQ ID NO: | CGCGGU (SEQ | GUGGAAAUGUAAAGAUCGU | CCGCGAACGA |
| | 44 | ID NO: 140) | UG (SEQ ID NO: 141) | CG (SEQ ID NO: |
| | | | | 142) |
| AP-00392 | SEQ ID NO: | CGUAGACGAU | CGUAGAAAUGUAAAGAUCG | GACUCGGUGU |
| | 52 | UUACCGGUC | UUG (SEQ ID NO: 144) | CUACU (SEQ ID |
| | | (SEQ ID NO: 143) | | NO: 145) |
| | SEQ ID NO: | X | CCCGGWGGGAUAGGAOUUA | Z |
| | 38 | | UCGG (SEQ ID NO: 102) | |
| AP- | SEQ ID NO: | GCAA_(SEQ ID | CCCGGAGGGAUAGGAAUUA | UUGC (SEQ ID |
| 00341; | 41 | NO: 146) | UCGG (SEQ ID NO: 147) | NO: 148) |
| AP-00248 | | | | |
| AP- | SEQ ID NO: | UAUACGCAA | CCCGGUGGGAUAGGAUUAU | UUGCAUAAGC |
| 00030; | 15 | (SEQ ID NO: 149) | CGG (SEQ ID NO: 150) | CAUUGU (SEQ |
| AP- | | | | ID NO: 151) |
| 00039; | | | | |
| AP-00242 | | | | |
| AP- | SEQ ID NO: | UCGACAGA | CCCGGAGGGAUAGGAUUAU | UCUUGAGAUG |
| 00031; | 16 | (SEQ ID NO: 152) | CGG (SEQ ID NO: 153) | CAAACUU (SEQ |
| AP- | | | | ID NO: 154) |
| 00040; | | | | |
| AP-00243 | | | | |
| AP- | SEQ ID NO: | UCCCAUAGAU | CCCGGAGGGAUAGGAAUUA | AUCUAAACGU |
| 00032; | 17 | (SEQ ID NO: 155) | UCGG (SEQ ID NO: 156) | CUGU (SEQ ID |
| AP- | | | | NO: 157) |
| 00041; | | | | |
| AP-00244 | | | | |
| AP- | SEQ ID NO: | UUGGGCGUCU | CCCGGUGGGAUAGGAUUAU | UCUU (SEQ ID |
| 00034; | 19 | CGCCAUUAUG | CGG (SEQ ID NO: 159) | NO: 160) |
| AP- | | A (SEQ ID NO: | | |
| 00043; | | 158) | | |
| AP-00245 | | | | |
| AP-00246 | SEQ ID NO: | GCAA (SEQ ID | CCCGGUGGGAUAGGA- | UUGC (SEQ ID |
| | 39 | NO: 161) | UUAUCG (SEQ ID NO: 162) | NO: 163) |
| AP-00247 | SEQ ID NO: | GCAA (SEQ ID | CCCGGAGGGAUAGGA- | UUGC (SEQ ID |
| | 40 | NO: 164) | UUAUCGG (SEQ ID NO: 165) | NO: 166) |

*Aptamer sequence: 5′-stem region (X) - Core Sequence - 3′-stem region (Z)

### Aptamer Secondary and Tertiary Structure

Aptamer binding to a target is mediated by the three-dimensional structure of the aptamer, which is determined by its sequence of nucleosides. Interactions between nucleosides within a single aptamer molecule cause the aptamer to fold to form secondary structural elements, which in turn generate the tertiary structure of the aptamer.

When used herein in relation to aptamers, the terms “interact” and “interaction” refer to associations between nucleosides mediated by hydrogen bonding between their nucleobases. For example, an interaction may be mediated by canonical (Watson-Crick) base pairing, which is based on the formation of hydrogen bonds between (i) an adenine nucleobase and a thymine or uracil nucleobase, or (ii) a cytosine nucleobase and a guanine nucleobase. Nucleosides that interact via canonical base pairing are also described herein as being “complementary”. Interactions may also be mediated by non-canonical base pairing, which is based on planar hydrogen-bonding pairs of nucleobases having hydrogen bonding patterns that differ from those of canonical base pairs. The most common non-canonical base pair is the guanine-uracil Wobble base pair.

Various software applications for computational prediction of the secondary structure of single-stranded nucleic acids based on their nucleic acid sequence are known to the skilled person. For example, aptamer secondary structure can be computationally predicted using the current version of the mfold algorithm available via a web server, as described for example in Zuker, Nucleic Acids Res (2003), 31 (13), 3406-3415. The mfold algorithm results in one or several ‘optimal’ and ‘suboptimal’ structures for a given nucleobase sequence. Accordingly, in some instances of the aptamers disclosed herein, the nucleosides that interact to form a stem structure in a computationally predicted secondary structure of the folded aptamer are identified using a software application, such as mfold or RNAfold. In some instances, the nucleosides that interact to form a stem structure in a computationally predicted secondary structure of the folded aptamer are identified based on a computationally predicted secondary structure identified using mfold. In some such instances, mfold-predicted secondary structures are identified using a percent suboptimality number of 50, as described in Example 18 herein. If the percent suboptimality number is set to 50, then only predicted secondary structures that are within 50% of the minimum free energy (MFE) will be computed. As demonstrated in Example 18, mfold may return multiple predicted structures that are within 50% of the MFE. Where a computational prediction of the secondary structure of an aptamer returns multiple predicted structures, “a computationally predicted secondary structure” refers to any one or more of these predicted structures.

Accordingly, in some instances of the aptamers disclosed herein, the nucleosides that interact to form a stem structure in a computationally predicted secondary structure of the folded aptamer are identified using mfold software, wherein the mfold-predicted secondary structure is identified using a percent suboptimality number of 50.

Certain nucleosides of the aptamers disclosed herein are identified as interacting to form a “stem structure” in the computationally predicted secondary structure of the folded aptamer. This stem structure in the folded aptamer is formed by the interaction of two non-contiguous nucleoside regions of the aptamer (stem-forming regions) to form an antiparallel double-stranded region. In the folded aptamers disclosed herein the two stem-forming regions in the computationally predicted secondary structure of the folded aptamer are separated by an interposed region of linked nucleosides.

A stem structure may include one or more bulges. As used herein, the term “bulge” refers to a stretch of one or more nucleosides within a stem-forming region whose nucleobases do not interact with any of the nucleobases of the other stem-forming region of the aptamer in the computationally predicted secondary structure of the folded aptamer despite their location within a stem-forming region.

### Folded Aptamers

Disclosed herein are compounds comprising folded, functional aptamers that bind to the human transferrin receptor 1 (hTfR1) and does not compete with human transferrin (hTf) for binding to hTfR1.

Disclosed herein is an aptamer that binds to the human transferrin receptor 1 (hTfR1) and does not compete with human transferrin (hTf) for binding to hTfR1, wherein the aptamer comprises the sequence of linked nucleosides:

(SEQ ID NO: 71)
5′-XYGURGAAAKGUAAMGAUCRUUGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
Y is a nucleoside comprising a C or U nucleobase,
R is a nucleoside comprising an A or G nucleobase,
K is a nucleoside comprising a G or U nucleobase,
M is a nucleoside comprising an A or C nucleobase, and
X and Z are each independently a sequence of 4 to 12 linked nucleosides comprising A, C, G or U nucleobases;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X and Z are the same length as each other. In some instances, X and Z have different lengths. In some such instances, the nucleoside sequence of X is the reverse complement of the nucleoside sequence of Z.

In some instances, this aptamer comprises the sequence of linked nucleosides:

(SEQ ID NO: 56)
5′-X₁UUX₂YGURGAAAKGUAAMGAUCRUUGZ₂Z₁-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
Y is a nucleoside comprising a C or U nucleobase,
R is a nucleoside comprising an A or G nucleobase,
K is a nucleoside comprising a G or U nucleobase,
M is a nucleoside comprising an A or C nucleobase,
X₁ and Z₁ are each independently a sequence of 3 to 9 linked nucleosides comprising A, C, G or U nucleobases and X₁ and Z₁, and
X₂ and Z₂ are each independently a nucleoside comprising an A, C, G or U nucleobase;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X₁ and Z₁ are the same length as each other. In some instances, X₁ and Z₁ have lengths different from each other. In some such instances, the nucleoside sequence of X₁ is the reverse complement of the nucleoside sequence of Z₁ and X₂ is complementary to Z₂.

In some instances, this aptamer comprises one of the sequences of linked nucleosides of SEQ ID NO: 48, SEQ ID NO: 49, SEQ ID NO: 50, SEQ ID NO: 53 or SEQ ID NO: 54 as set forth in Table 15; SEQ ID NO: 57 or SEQ ID NO: 58 as disclosed in Example 5B; or SEQ ID NO: 64 as set forth in Table 19.

In some instances, this aptamer comprises the sequence of linked nucleosides

(SEQ ID NO: 53)
5′-CGGACUUACGUAGAAAGGUAACGAUCAUUGUGUCCG-3′.

In some instances, the nucleobase sequence of this aptamer consists of the sequence 5′-CGGACUUACGUAGAAAGGUAACGAUCAUUGUGUCCG-3′ (SEQ ID NO: 53).

In some instances, the nucleobase sequence of this aptamer consists of one the sequences of linked nucleosides of SEQ ID NO: 48, SEQ ID NO: 49, SEQ ID NO: 50, SEQ ID NO: 53 or SEQ ID NO: 54 as set forth in Table 15; SEQ ID NO: 57 or SEQ ID NO: 58 as disclosed in Example 5B; or SEQ ID NO: 64 as set forth in Table 19.

Also disclosed herein is an aptamer that binds to the human transferrin receptor 1 (hTfR1) and which does not compete with human transferrin (hTf) for binding to hTfR1, wherein the aptamer comprises the sequence of linked nucleosides:

(SEQ ID NO: 43)
5′-XUGURGAAAKGUAAMGAUCGUUGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
R is a nucleoside comprising an A or G nucleobase,
K is a nucleoside comprising a G or U nucleobase,
M is a nucleoside comprising an A or C nucleobase, and
X and Z are each independently a sequence of 4 or 5 linked nucleosides comprising A, C, G or U nucleobases;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X and Z are the same length as each other. In some instances, X and Z have different lengths. In some such instances, the nucleoside sequence of X is the reverse complement of the nucleoside sequence of Z.

In some instances, this aptamer comprises one of the sequences of linked nucleosides of SEQ ID NO: 26, SEQ ID NO: 27, or SEQ ID NO: 37 as set forth in Table 12; SEQ ID NO: 27, SEQ ID NO: 44, or SEQ ID NO: 45 as set forth in Table 13; SEQ ID NO: 37, SEQ ID NO: 45, or SEQ ID NO: 52 as set forth in Table 15.

In some instances, this aptamer comprises the sequence of linked nucleosides

(SEQ ID NO: 45)
5′-CGCGGUGUGGAAAUGUAAAGAUCGUUGCCGCG-3′.

In some instances, the nucleobase sequence of this aptamer consists of the sequence 5′-CGCGGUGUGGAAAUGUAAAGAUCGUUGCCGCG-3′ (SEQ ID NO: 45).

In some instances, the nucleobase sequence of this aptamer consists of one of the sequences of linked nucleosides of SEQ ID NO: 26, SEQ ID NO: 27, or SEQ ID NO: 37 as set forth in Table 12; SEQ ID NO: 27, SEQ ID NO: 44, or SEQ ID NO: 45 as set forth in Table 13; SEQ ID NO: 37, SEQ ID NO: 45, or SEQ ID NO: 52 as set forth in Table 15.

Also disclosed herein is an aptamer that binds to the human transferrin receptor 1 (hTfR1) and which does not compete with human transferrin (hTf) for binding to hTfR1, wherein the aptamer comprises the sequence of linked nucleosides:

(SEQ ID NO: 38)
5′-XCCCGGWGGGAUAGGAOUUAUCGGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
W is a nucleoside comprising an A or U nucleobase,
O is a nucleoside comprising an A nucleobase or an absent nucleoside, and
X and Z are each independently a sequence of 2 to 5 linked nucleosides comprising A, C, G or U nucleobases;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X and Z are the same length as each other. In some instances, X and Z have different lengths. In some such instances, the nucleoside sequence of X is the reverse complement of the nucleoside sequence of Z.

In some instances, this aptamer comprises one of the sequences of linked nucleosides of SEQ ID NO: 15, SEQ ID NO: 16, or SEQ ID NO: 17, SEQ ID NO: 19 as set forth in Table 10; or SEQ ID NO: 15, SEQ ID NO: 39, or SEQ ID NO: 40, or SEQ ID NO: 41 as set forth in Table 11.

In some instances, this aptamer comprises the sequence of linked nucleosides

(SEQ ID NO: 41)
5′-GCAACCCGGAGGGAUAGGAAUUAUCGGUUGC-3′.

In some instances, the nucleobase sequence of this aptamer consists of the sequence

(SEQ ID NO: 41)
5′-GCAACCCGGAGGGAUAGGAAUUAUCGGUUGC-3′.

In some instances, the nucleobase sequence of this aptamer consists of one of the sequences of linked nucleosides of SEQ ID NO: 15, SEQ ID NO: 16, or SEQ ID NO: 17, SEQ ID NO: 19 as set forth in Table 10; or SEQ ID NO: 15, SEQ ID NO: 39, or SEQ ID NO: 40, or SEQ ID NO: 41 as set forth in Table 11.

### Unfolded Aptamers

Also disclosed herein are compounds comprising the unfolded version of each of the (folded, functional) aptamers disclosed herein. Unfolded versions of the aptamers disclosed herein may be generated, for example, during the manufacture, purification, storage and/or testing of the aptamers or aptamer conjugates disclosed herein, and unfolded versions of the aptamers may be advantageous in some contexts (such as in the manufacture, purification, storage and/or testing of the aptamers disclosed herein), but they do not benefit from the surprisingly advantageous combinations of properties that enhance the suitability of the aptamers and aptamer conjugates disclosed herein for their intended uses.

Unfolded aptamers may be generated, for example, by exposing a folded aptamer to denaturing conditions, such as high temperature (e.g., by heating the aptamer at 70° C. for 3 minutes or at 65° C. for 5 minutes).

Thus, disclosed herein is an unfolded aptamer which comprises the sequence of linked nucleosides:

(SEQ ID NO: 71)
5′-XYGURGAAAKGUAAMGAUCRUUGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
Y is a nucleoside comprising a C or U nucleobase,
R is a nucleoside comprising an A or G nucleobase,
K is a nucleoside comprising a G or U nucleobase,
M is a nucleoside comprising an A or C nucleobase, and
X and Z are each independently a sequence of 4 to 12 linked nucleosides comprising A, C, G or U nucleobases;
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X and Z are the same length as each other. In some such instances, the nucleoside sequence of X is the reverse complement of the nucleoside sequence of Z.

Also disclosed herein is an unfolded aptamer that comprises the sequence of linked nucleosides:

(SEQ ID NO: 56)
5′-X₁UUX₂YGURGAAAKGUAAMGAUCRUUGZ₂Z₁-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
Y is a nucleoside comprising a Cor U nucleobase,
R is a nucleoside comprising an A or G nucleobase,
K is a nucleoside comprising a G or U nucleobase,
M is a nucleoside comprising an A or C nucleobase,
X₁ and Z₁ are each independently a sequence of 3 to 9 linked nucleosides comprising A, C, G or U nucleobases,
X₂ and Z₂ are each independently a nucleoside comprising an A, C, G or U nucleobase,
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X₁ and Z₁ are the same length as each other. In some instances, X and Z have different lengths. In some such instances, the nucleoside sequence of X₁ is the reverse complement of the nucleoside sequence of Z₁ and X₂ is complementary to Z₂.

Also disclosed herein is an unfolded aptamer that comprises the sequence of linked nucleosides 5′-CGGACUUACGUAGAAAGGUAACGAUCAUUGUGUCCG-3′ (SEQ ID NO: 53).

Also disclosed herein is an unfolded aptamer wherein the nucleobase sequence of the aptamer consists of the sequence 5′-CGGACUUACGUAGAAAGGUAACGAUCAUUGUGUCCG-3′ (SEQ ID NO: 53).

Disclosed herein is an unfolded aptamer which comprises the sequence of linked nucleosides:

(SEQ ID NO: 43)
5′-XUGURGAAAKGUAAMGAUCGUUGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
R is a nucleoside comprising an A or G nucleobase,
K is a nucleoside comprising a G or U nucleobase,
M is a nucleoside comprising an A or C nucleobase, and
X and Z are each independently a sequence of 4 or 5 linked nucleosides comprising A, C, G or U nucleobases,
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X and Z are the same length as each other. In some instances, X and Z have different lengths. In some such instances, the nucleoside sequence of X is the reverse complement of the nucleoside sequence of Z.

Also disclosed herein is an unfolded aptamer that comprises the sequence of linked nucleosides 5′-CGCGGUGUGGAAAUGUAAAGAUCGUUGCCGCG-3′ (SEQ ID NO: 45).

Also disclosed herein is an unfolded aptamer wherein the nucleobase sequence of the aptamer consists of the sequence 5′-CGCGGUGUGGAAAUGUAAAGAUCGUUGCCGCG-3′ (SEQ ID NO: 45).

Disclosed herein is an unfolded aptamer which comprises the sequence of linked nucleosides:

(SEQ ID NO: 38)
5′-XCCCGGWGGGAUAGGAOUUAUCGGZ-3′,

wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
W is a nucleoside comprising an A or U nucleobase,
O is a nucleoside comprising an A nucleobase or an absent nucleoside, and
X and Z are each independently a sequence of 2 to 5 linked nucleosides comprising A, C, G or U nucleobases,
wherein the guanosine nucleosides of the aptamer each comprise a 2′-fluoro-modified ribose sugar moiety, and the adenosine nucleosides, the cytosine nucleosides and the uridine nucleosides of the aptamer each comprise a 2′-O-methyl-modified ribose sugar moiety, and
wherein all of the nucleosides of the aptamer are connected by phosphodiester internucleoside linkages.

In some instances, X and Z are the same length as each other. In some instances, X and Z have different lengths. In some such instances, the nucleoside sequence of X is the reverse complement of the nucleoside sequence of Z.

Also disclosed herein is an unfolded aptamer that comprises the sequence of linked nucleosides 5′-GCAACCCGGAGGGAUAGGAAUUAUCGGUUGC-3′ (SEQ ID NO: 41).

Also disclosed herein is an unfolded aptamer wherein the nucleobase sequence of the aptamer consists of the sequence 5′-GCAACCCGGAGGGAUAGGAAUUAUCGGUUGC-3′ (SEQ ID NO: 41).

### Aptamer Synthesis

The aptamers disclosed herein can be produced using any suitable known method of oligonucleotide synthesis. The aptamers disclosed herein can be produced by chemical synthesis or enzymatic synthesis. Aptamers may be synthesized using the aptamer synthesis method described in Example 12 herein.

Aptamers can be produced by solid phase chemical synthesis. Oligonucleotide solid phase chemical synthesis is carried out by a stepwise addition of one nucleotide residue to the 5′ terminus of the growing chain that is bound to a solid support. Each addition step consists of four chemical reactions: (1) de-blocking the support-bound oligonucleotide precursor, (2) coupling of a nucleoside phosphoramidite, (3) capping any unreacted 5′ hydroxyl groups on the support-bound precursor, and (4) oxidation of the phosphite-triester formed in the coupling step. This process is repeated, once for each nucleoside, to produce the required oligonucleotide.

### Aptamer Length

Aptamers identified using SELEX screening are commonly 60-100 nt in length. However, it is desirable to reduce aptamer length to increase manufacturing efficiency and reduce costs, as well as to reduce the chance of off-target interactions.

Aptamers can be produced by solid phase chemical synthesis. The yield of this synthetic process is highly dependent on the length of the aptamer and the average stepwise yield (coupling efficiency). Even with high coupling efficiencies the yield rapidly decreases as oligonucleotide length increases. While coupling efficiencies of up to 99.5% can be reached during solid phase chemical synthesis, it is not possible to achieve 100% reaction of the support-bound precursor with the incoming phosphoamidite.

The expected yield of solid phase chemical synthesis of an oligonucleotide can be calculated as yield=C^{(N-1)}, where C is the coupling efficiency and N is the oligonucleotide length. For example, for a chemical synthesis reaction with a coupling efficiency of 99.5%, the expected yield would be 95.6% for a 10-base oligonucleotide, 90.9% for a 20-base oligonucleotide, 86.5% for a 30-base oligonucleotide and only 78.2% for a 50-base oligonucleotide. The disadvantages of a having low yield are two-fold; in addition to there being a lower amount of the desired product, there is a higher amount of the contaminating shorter oligonucleotides that result from the failed coupling steps, which creates a heterogeneous mixture of molecules which is generally undesirable at least for therapeutic and diagnostic uses and makes the desired, full-length product more difficult to purify. It is therefore important to maximize the yield of an aptamer chemical synthesis process. One way to achieve this is by minimizing the length of the aptamer, while retaining its binding functionality.

The aptamers disclosed herein may be between 25 and 50 nucleosides in length. In some instances, the aptamer is 25-40 nucleosides in length.

The minimum length of the SEQ ID NO: 56 consensus sequence is 32 nucleosides, and the maximum length is 44 nucleosides. The minimum length of the SEQ ID NO: 43 consensus sequence is 30 nucleosides, and the maximum length is 32 nucleosides. The minimum length of the SEQ ID NO: 38 consensus sequence is 26 nucleosides, and the maximum length is 33 nucleosides. Accordingly, in some instances, the aptamer is 26-44 nucleosides in length.

In some instances, the aptamer comprises the nucleobase sequence SEQ ID NO: 56 consensus sequence and the aptamer is 32-44 nucleosides in length. In some instances, the aptamer comprises the SEQ ID NO: 43 consensus sequence and the aptamer is 30-32 nucleosides in length. In some instances, the aptamer comprises the SEQ ID NO: 38 consensus sequence and the aptamer is 26-44 nucleosides in length.

SEQ ID NO: 53 is 36 nucleosides in length. SEQ ID NO: 45 is 32 nucleosides in length. SEQ ID NO: 41 is 31 nucleosides in length. Accordingly, in some instances the aptamer is 30-37 nucleosides in length, or 31-36 nucleotides in length, or 31 nucleotides in length, or 32 nucleotides in length, or 36 nucleotides in length.

In some instances, the aptamer is less than 40 nucleosides in length. In some instances, the aptamer is less than 37 nucleosides in length.

### Nucleoside Modifications

In some embodiments, the aptamer is chemically modified to improve their binding affinity and/or nuclease stability.

In some embodiments, the aptamers disclosed herein comprise chemically modified nucleosides. For example, the aptamer can comprise an oligonucleotide modification selected from the group consisting of internucleotide linkage modification, sugar modification, nucleobase modification, and any combinations thereof.

In some embodiments, the guanosine nucleosides of the aptamer each comprise a 2′-fluoro-modified ribose sugar moiety. In some embodiments, the adenosine nucleosides, the cytosine nucleosides and the uridine nucleosides of the aptamer each comprise a 2′-O-methyl-modified ribose sugar moiety. In some embodiments, one or more guanosine nucleosides of the aptamer comprise a 2′-fluoro-modified ribose sugar moiety. In some embodiments, one or more adenosine nucleosides of the aptamer comprise a 2′-O-methyl-modified ribose sugar moiety. In some embodiments, one or more cytosine nucleosides of the aptamer comprise a 2′-O-methyl-modified ribose sugar moiety. In some embodiments, one or more uridine nucleosides of the aptamer comprise a 2′-O-methyl-modified ribose sugar moiety. In some embodiments, one or more adenosine nucleosides, the cytosine nucleosides and the uridine nucleosides of the aptamer comprise a 2′-O-methyl-modified ribose sugar moiety.

By “2′-fluoro-modified ribose sugar moiety” herein is meant an RNA ribose sugar moiety that contains a fluorine substituent at the 2′ ribose position (in place of the 2′-hydroxyl group in an unmodified RNA ribose sugar moiety) but which is otherwise unmodified.

By “2′-O-methyl-modified ribose sugar moiety” herein is meant an RNA ribose sugar moiety that contains a methoxy (—OCH₃) substituent at the 2′ ribose position (in place of the 2′-hydroxyl group in an unmodified RNA ribose sugar moiety) but which is otherwise unmodified.

In some embodiments, the aptamer comprises pyrimidine nucleosides (i.e. the cytosine and uridine nucleosides) each comprising a 2′-fluoro-modified ribose sugar moiety and the purine nucleosides (i.e. the guanosine and adenosine nucleosides) each comprising an unmodified ribose sugar moiety having a 2′-hydroxyl group.

In some embodiments, the aptamer comprises a nucleotide analog, such as first-generation bridged nucleic acids (BNA), also known as locked nucleic acids (LNA), second-generation BNA, BNANC (2′-O,4′-aminoethylene bridged nucleic acid). In some embodiments, the aptamer comprises 2′-deoxy-2′-fluoro-beta-D-arabinonucleic acid (2′F-ANA) and/or 2′-O-methoxy-ethyl Bases (2′-MOE).

### The 5′- and 3′ Ends of the Aptamers

In some instances, any of the aptamers disclosed herein may be generated with a 3′ inverted deoxythymine (dT). The resulting 3′-3′ linkage between adjacent nucleosides at the 3′ end of the aptamer inhibits degradation by 3′ exonucleases, enhancing aptamer stability.

In some instances, any of the aptamers disclosed herein may be generated with a 5′ Thiol-Modifier C₆S—S(1-O-Dimethoxytrityl-hexyl-disulfide, 1′-[(2-cyanoethyl)-(N,N-diisopropyl)]-phosphoramidite), to facilitate conjugations.

In some instances, any of the aptamers disclosed herein may be generated with a 5′ hexylamine linker (C₆NH₂).

### Aptamer Stability

### Serum Stability

Aptamer stability in serum may affect the ability of the aptamer to localize to tumors or other in vivo target sites. In some embodiment, the aptamers disclosed herein benefit from improved in vivo stability relative to known hTfR aptamers by comprising some or all of the sugar moieties of the nucleosides in the aptamer which are chemically modified. In some embodiments, the aptamer conjugates disclosed herein benefit from improved in vivo stability relative to equivalent aptamer conjugates based on known hTfR aptamers. The statements regarding improved stability below apply equally to the aptamers disclosed herein, the compounds disclosed herein, and the aptamer conjugates disclosed herein.

The aptamers disclosed herein may demonstrate stability in an appropriate in vitro serum stability assay. For example, the aptamers disclosed herein may demonstrate a half-life of at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, in a serum stability assay. Aptamers disclosed herein may demonstrate a half-life of at least 12 hours in a serum stability assay. Aptamers disclosed herein may demonstrate a half-life of at least 18 hours in a serum stability assay. Aptamers disclosed herein may demonstrate a half-life of at least 24 hours in a serum stability assay. Aptamers disclosed herein may demonstrate a half-life of at least 48 hours in a serum stability assay. Aptamers disclosed herein may demonstrate a half-life of at least 96 hours in a serum stability assay.

In some embodiments, the aptamers disclosed herein demonstrate improved stability in an in vitro serum stability assay, relative to known hTfR aptamers, such as the known hTfR1-binding aptamers Waz (SEQ ID NO: 60), C2 (SEQ ID NO: 65) and C2.min (SEQ ID NO: 66). For example, the aptamers disclosed herein may demonstrate a half-life that is at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, or at least 2.0-fold the half-life of a known hTfR aptamer (such as the Waz, C2 or C2.min aptamer) in an in vitro serum stability assay.

Serum stability of the aptamers disclosed herein may be determined using an in vitro serum stability assay that comprises incubation of an aptamer in fresh mammalian serum, such as fresh mouse serum. The serum stability of aptamers may be determined using a serum stability assay of the type described in Kelly et al. (2021) and in Example 13 herein.

In some embodiments, in this serum stability assay the aptamers disclosed herein will display half-lives greater than 8 hours and that those displaying the longest half-life display a half-life of greater than 10 hours, greater than 12 hours, greater than 18 hours, greater than or 24 hours, greater than 36 days, and perhaps greater than 48 hours and greater than 96 hours.

### Media Stability

It may be useful to assess the stability of the aptamers in media to ensure that degradation of the aptamers during the course of an assay is not confounding the results of that assay, especially for assays that involve longer timepoints. A suitable media stability assay is described in Example 14.

In some embodiments, the aptamers, compounds, and aptamer conjugates disclosed herein are stable when tested in this media stability assay, and would show little to no degradation at 24, 48 or 96 hours (for example >>95% stability at 24 hours, >95% stability at 48 hours, or >95% stability at 96 hours).

### Aptamer Binding

Some aptamers disclosed herein bind to the human transferrin receptor 1 (hTfR1) and do not compete with human transferrin (hTf) for binding to hTfR1.

By “bind to the human transferrin receptor 1 (hTfR1)” herein (and equivalent expressions) is meant that the aptamer demonstrates an apparent binding constant (K_{D}) for hTfR1-expressing cells of less than 500 nmol/l (nM) and shows at least 10 times greater staining of hTfR1-expressing cells compared to the untreated cells when determined using the Jurkat cell binding assay described in Maier et al. (2016) or an apparent binding constant (K_{D}) of less than 500 nmol/l (nM) as determined using the bio-layer interferometry assay of Example 11 herein.

The assay described in Maier et al. (2016) involves cell staining using increasing concentrations of labeled aptamers. Treated cell populations are subsequently analyzed using flow cytometry and the median fluorescence intensity used to determine the apparent binding constants for the aptamers.

Analogous considerations apply to any references herein to aptamer that “binds to transferrin receptor 1 (TfR1) on the surface of cells of a non-human primate (NHP)” (and equivalent expressions). Such aptamers demonstrate an apparent binding constant (K_{D}) for NHP TfR1-expressing cells of less than 500 nmol/l in a binding assay of the type described in Maier et al. (2016) using NHP cells or an apparent binding constant (K_{D}) of less than 500 nmol/l (nM) as determined using a bio-layer interferometry assay of the type described in Example 11 herein and NHP TfR1.

By “does not compete with human transferrin (hTf) for binding to hTfR1” herein (and equivalent expressions) is meant that the aptamer does not demonstrate significant competition with hTf for binding to hTfR1, as assessed using an appropriate competition assay. Appropriate competition assays include, for example, competitive binding and cellular uptake assays in which fluorescently labeled aptamers compete with hTf for binding to hTfR1 and in which cellular uptake of the fluorescently labeled aptamers is assessed by flow cytometry. One such appropriate assay is described in Maier et al. (2016). In the previously described assay, chemically synthesized, fluorescently labeled (Dylight650) aptamers were incubated with Jurkat cells in the absence or the presence of 25 μmol/l differic Tf, and binding and uptake of the aptamers was assessed using flow cytometry. Appropriate competition assays may involve additional control experiments, to enable comparisons beyond the hTf-treatment-positive and hTf-treatment-negative aptamer binding comparison, in which the aptamer of interest is incubated in the presence of other potential competitors for hTfR1 binding. These additional control experiments may involve, for example, competition between a fluorescently labeled aptamer of interest and an excess (such as a 10-fold excess) of an unlabelled version of the aptamer of interest (to confirm the expected inhibition of the aptamer by competition with itself) and/or competition between a fluorescently labeled aptamer of interest and a negative control aptamer that does not bind to hTfR1.

Analogous considerations apply to any references herein to aptamers that “do not compete with transferrin of an NHP for binding to NHP TfR1” (and equivalent expressions). Such aptamers do not demonstrate significant competition with NHP Tf for binding to NHP TfR1, as assessed using a corresponding appropriate competition assay.

### Human Transferrin Receptor 1

hTFR1 (also known as CD71) is a cell membrane-associated glycoprotein involved in the cellular uptake of iron and in the regulation of cell growth. Delivery and uptake of iron into cells occurs through the binding of iron-loaded transferrin (Tf) to hTfR1 and its subsequent internalization. hTfR1 is also constitutively internalized independently of ligand binding.

The amino acid sequences of hTfR1 and human transferrin (hTf) are provided below:

hTfR1 amino acid sequence
(SEQ ID NO: 67; UniProt P02786)
MMDQARSAFSNLFGGEPLSYTRFSLARQVDGDNSHVEMKLAVDEEENADN
NTKANVTKPKRCSGSICYGTIAVIVFFLIGFMIGYLGYCKGVEPKTECER
LAGTESPVREEPGEDFPAARRLYWDDLKRKLSEKLDSTDFTGTIKLLNEN
SYVPREAGSQKDENLALYVENQFREFKLSKVWRDQHFVKIQVKDSAQNSV
IIVDKNGRLVYLVENPGGYVAYSKAATVTGKLVHANFGTKKDFEDLYTPV
NGSIVIVRAGKITFAEKVANAESLNAIGVLIYMDQTKFPIVNAELSFFGH
AHLGTGDPYTPGFPSFNHTQFPPSRSSGLPNIPVQTISRAAAEKLFGNME
GDCPSDWKTDSTCRMVTSESKNVKLTVSNVLKEIKILNIFGVIKGFVEPD
HYVVVGAQRDAWGPGAAKSGVGTALLLKLAQMFSDMVLKDGFQPSRSIIF
ASWSAGDFGSVGATEWLEGYLSSLHLKAFTYINLDKAVLGTSNFKVSASP
LLYTLIEKTMQNVKHPVTGQFLYQDSNWASKVEKLTLDNAAFPFLAYSGI
PAVSFCFCEDTDYPYLGTTMDTYKELIERIPELNKVARAAAEVAGQFVIK
LTHDVELNLDYERYNSQLLSFVRDLNQYRADIKEMGLSLQWLYSARGDFF
RATSRLTTDFGNAEKTDRFVMKKLNDRVMRVEYHFLSPYVSPKESPFRHV
FWGSGSHTLPALLENLKLRKQNNGAFNETLFRNQLALATWTIQGAANALS
GDVWDIDNEF
hTf amino acid sequence
(SEQ ID NO: 68; UniProt P02787)
MRLAVGALLVCAVLGLCLAVPDKTVRWCAVSEHEATKCQSFRDHMKSVIP
SDGPSVACVKKASYLDCIRAIAANEADAVTLDAGLVYDAYLAPNNLKPVV
AEFYGSKEDPQTFYYAVAVVKKDSGFQMNQLRGKKSCHTGLGRSAGWNIP
IGLLYCDLPEPRKPLEKAVANFFSGSCAPCADGTDFPQLCQLCPGCGCST
LNQYFGYSGAFKCLKDGAGDVAFVKHSTIFENLANKADRDQYELLCLDNT
RKPVDEYKDCHLAQVPSHTVVARSMGGKEDLIWELLNQAQEHFGKDKSKE
FQLFSSPHGKDLLFKDSAHGFLKVPPRMDAKMYLGYEYVTAIRNLREGTC
PEAPTDECKPVKWCALSHHERLKCDEWSVNSVGKIECVSAETTEDCIAKI
MNGEADAMSLDGGFVYIAGKCGLVPVLAENYNKSDNCEDTPEAGYFAVAV
VKKSASDLTWDNLKGKKSCHTAVGRTAGWNIPMGLLYNKINHCRFDEFFS
EGCAPGSKKDSSLCKLCMGSGLNLCEPNNKEGYYGYTGAFRCLVEKGDVA
FVKHQTVPQNTGGKNPDPWAKNLNEKDYELLCLDGTRKPVEEYANCHLAR
APNHAVVTRKDKEACVHKILRQQQHLFGSNVTDCSGNFCLFRSETKDLLF
RDDTVCLAKLHDRNTYEKYLGEEYVKAVGNLRKCSTSSLLEACTFRRP

### Non-Human Primate (NHP) Transferrin Receptor 1

Some of the aptamers disclosed herein benefit from an especially advantageous combination of stability and binding epitope on hTfR1. In particular, some of the particularly advantageous aptamers disclosed herein are aptamers wherein the aptamer (i) binds to hTfR1 on the surface of human cells without competing with hTf for binding to hTfR1 and is subsequently internalized by the human cells; and (ii) binds to TfR1 on the surface of cells of a NHP and is subsequently internalized by the NHP cells (and preferably also the aptamer does not compete with Tf of the NHP for binding to the NHP TfR1). These particularly advantageous TfR aptamers bind an epitope on hTfR1 that is shared between hTfR1 and the NHP TfR. Aptamers as disclosed herein with this combination of properties are especially advantageous because their binding to an epitope shared between hTfR1 and the NHP TfR renders them highly suitable for pre-clinical testing in animals, and in particular in NHPs, for example in NHP disease models. The use of such aptamers in pre-clinical testing can allow for the intended human drug product to be tested directly in NHPs. In contrast, in situations where an aptamer does not bind to an epitope shared between hTfR1 and the NHP TfR, it may not be possible for the intended human drug product to be tested directly in NHPs and that an alternative aptamer targeted to the NHP TfR would be required for pre-clinical testing in NHPs.

In Example 6, the inventors have shown that an anti-hTfR1 aptamer of SEQ ID NO: 41 binds to both hTfR1 and cynomolgus monkey (Macaca fascicularis) TfR1 (cTfR1) with similar affinities. In contrast, no binding was detected for AP-00074 (SEQ ID NO: 55) (an aptamer that corresponds to the known hTfR1 aptamer Waz with a single point mutation and which has equivalent hTfR1 binding characteristics to Waz).

Therefore, in some instances, the aptamer comprises the SEQ ID NO: 38 consensus sequence and the aptamer binds to TfR1 of an NHP. In some instances, the aptamer binds to TfR1 of a NHP and does not compete with Tf of the NHP for binding to the NHP TfR1. In some instances, the NHP is cynomolgus monkey (Macaca fascicularis).

The amino acid sequences of cynomolgus monkey TfR1 (cTfR1) and cynomolgus transferrin (cTf) are provided below:

cTfR1 amino acid sequence
(SEQ ID NO: 69; XP_045243212.1)
MMDQARSAFSNLFGGEPLSYTRFSLARQVDGDNSHVEMKLGVDEEENTDN
NTKANGTKPKRCGGNICYGTIAVIIFFLIGFMIGYLGYCKGVEPKTECER
LAGTESPAREEPEEDFPAAPRLYWDDLKRKLSEKLDTTDFTSTIKLLNEN
LYVPREAGSQKDENLALYIENQFREFKLSKVWRDQHFVKIQVKDSAQNSV
IIVDKNGGLVYLVENPGGYVAYSKAATVTGKLVHANFGTKKDFEDLDSPV
NGSIVIVRAGKITFAEKVANAESLNAIGVLIYMDQTKFPIVKADLSFFGH
AHLGTGDPYTPGFPSFNHTQFPPSQSSGLPNIPVQTISRAAAEKLFGNME
GDCPSDWKTDSTCKMVTSENKSVKLTVSNVLKETKILNIFGVIKGFVEPD
HYVVVGAQRDAWGPGAAKSSVGTALLLKLAQMFSDMVLKDGFQPSRSIIF
ASWSAGDFGSVGATEWLEGYLSSLHLKAFTYINLDKAVLGTSNFKVSASP
LLYTLIEKTMQDVKHPVTGRSLYQDSNWASKVEKLTLDNAAFPFLAYSGI
PAVSFCFCEDTDYPYLGTTMDTYKELVERIPELNKVARAAAEVAGQFVIK
LTHDTELNLDYERYNSQLLLFLRDLNQYRADVKEMGLSLQWLYSARGDFF
RATSRLTTDFRNAEKRDKFVMKKLNDRVMRVEYYFLSPYVSPKESPFRHV
FWGSGSHTLSALLESLKLRRQNNSAFNETLFRNQLALATWTIQGAANALS
GDVWDIDNEF
cTf amino acid sequence
(SEQ ID NO: 70; XP_005545850.2)
MRLAVGALLACAVLGLCLAVPEKSVRWCAVSEHEATKCQSFRDHMKSVLP
SDGPSVACVKKASHLDCIRAIAANEADAVTLDAGLVYDAYLAPNNLKPVV
AEFYGSKEDPQTFYYAVAVVKKESDFQMNQLRGRKSCHTGLGRSAGWNIP
IGLLYCDLPEPRKPLEKAVANFFSGSCVPCADGTDFPQLCQLCPGCGCST
LNQYFSYSGAFKCLKDGAGDVAFVKHSTIFENLANKADRDQYELLCLDNT
RKSVDEYKDCHLARVPSHTVVARSVGGKEDLIWELLNQAQEHFGKDKSKE
FQLFSSPHGKDLLFKDSAHGFLKVPPRMDAKMYLGYEYITAIRNLREGTC
PEAPTDECKPVKWCALSHHERLKCDEWSVNSAGKIECESAETTEDCIAKI
MNGEADAMSLDGGFVYIAGMCGLVPVLAENYNTPNPNCENTAEEGYFAVA
VVKSSSDLTWDNLKGKKSCHTAVGRTAGWNIPMGLLYSKINHCRFDEFFS
EGCAPGSEKNSSLCKLCMGPSPNLCEPNNKEGYYGYTGAFRCLVEKGDVA
FVKHQTVPQNTGGNNPDAWAKNLKEEDYELLCLDGSRKSVQEPANCHLAR
APNHAVVARKDKADCVQTLLLDQQRMFGSSVTDCSSNFCLFESKTKDLLF
RDDTVCLAKLHDRNTYEKYLGEEYVKAVTNLRKCSTSPLLEACTFHRA

### Binding Affinity

The aptamers disclosed herein bind to the human transferrin receptor 1 (hTfR1).

Aptamer binding affinity can be assessed using any suitable method known in the art. For example, the binding constant (K_{D}) may be determined using bio-layer interferometry (BLI), as taught in Example 11 herein. The binding constant can be determined using the equation K_{D}=kₒₙ/k_{off}.

In some embodiments, the aptamer conjugates disclosed herein bind to hTfR1 with the same or similar affinity to the corresponding non-conjugated aptamers. The statements regarding binding affinity below therefore apply equally to the aptamers disclosed herein, the compounds disclosed herein, and the aptamer conjugates disclosed herein.

In some instances, the aptamers disclosed herein have a binding constant (K_{D}) of less than 300 nmol/l (nM) as determined using bio-layer interferometry (BLI). In some instances, the aptamers disclosed herein have a binding constant (K_{D}) of less than 150 nmol/l (nM) as determined using bio-layer interferometry (BLI). In some instances, the aptamers disclosed herein have a binding constant (K_{D}) of less than 100 nmol/l (nM) as determined using bio-layer interferometry (BLI). In some instances, the aptamers disclosed herein have a binding constant (K_{D}) of less than 75 nmol/l (nM) as determined using bio-layer interferometry (BLI). In some instances, the aptamers disclosed herein have a binding constant (K_{D}) of less than 50 nmol/l (nM) as determined using bio-layer interferometry (BLI).

In some uses of the aptamers, a stronger binding affinity (lower binding constant) may be preferable. Some aptamers disclosed herein have a stronger binding affinity for hTfR1 than known hTfR1 aptamers.

In some such instances, the aptamer comprises the SEQ ID NO: 56 consensus sequence and has a binding constant of less than 60 nmol/l (nM) as determined using bio-layer interferometry (BLI).

In some such instances, the aptamer comprises SEQ ID NO: 53 or SEQ ID NO: 54 and has a binding constant of less than 30 nmol/l (nM) as determined using bio-layer interferometry (BLI).

In some such instances, the aptamer comprises SEQ ID NO: 53 and has a binding constant of less than 15 nmol/l (nM) as determined using bio-layer interferometry (BLI).

In some other uses of the aptamers, a weaker binding affinity (higher binding constant) may be preferable. For example, in applications relating to delivery of aptamers across the blood brain barrier (BBB), a weaker binding affinity can be beneficial for facilitating release of the aptamer in the brain after it has crossed the BBB. Therefore, in some instances, the aptamers have a weaker binding affinity for hTfR1 relative to known hTfR1 aptamers.

In some such instances, the aptamer comprises the SEQ ID NO: 43 consensus sequence and has a binding constant of 200-400 nmol/l (nM) as determined using bio-layer interferometry (BLI). In some such instances, the aptamer comprises SEQ ID NO: 45 and has a binding constant of less than 250-300 nmol/l (nM) as determined using bio-layer interferometry (BLI).

In some instances, the aptamer comprises the SEQ ID NO: 38 consensus sequence and has a binding constant of 100-300 nmol/l (nM) as determined using bio-layer interferometry (BLI). In some such instances, the aptamer comprises SEQ ID NO: 41 and has a binding constant of less than 100-150 nmol/l (nM) as determined using bio-layer interferometry (BLI).

### Binding Specificity

In some embodiments, the aptamers disclosed herein demonstrate hTfR1-specific binding, and hTfR1-specific cellular uptake (particularly, when hTfR1 is expressed natively on human cells). The aptamers disclosed herein may additionally demonstrate NHP TfR1-specific binding, and NHP TfR1-specific cellular uptake (particularly, when an NHP TfR1, such as cynomolgus monkey TfR1 (cTfR1), is expressed natively on NHP cells). Accordingly, the aptamers disclosed herein may advantageously demonstrate both hTfR1-specific binding and hTfR1-specific cellular uptake when hTfR1 is expressed natively on human cells and NHP TfR1-specific binding and NHP TfR1-specific cellular uptake when an NHP TfR1, such as cynomolgus monkey TfR1 (cTfR1), is expressed natively on NHP cells.

By “hTfR1-specific binding” or “NHP TfR1-specific binding” (and equivalent expressions herein) is meant epitope-specific binding of the aptamer to hTfR1 or NHP TfR1 rather than non-specific interactions with, for example, proteins or lipids on the cell surface through charge-charge interactions.

By “hTfR1-specific cellular uptake” or “NHP TfR1-specific cellular uptake” (and equivalent expressions herein) is meant internalization of the aptamer consequent to hTfR1-specific binding or NHP TfR1-specific binding of the aptamer, rather than non-specific uptake into cells as a result of non-specific interactions of the aptamer with, for example, proteins or lipids on the cell surface through charge-charge interactions.

Identification of aptamers that benefit from hTfR-specific binding and hTfR-specific cellular uptake may be identified by using one or more ‘internalization and binding with blocking’, ‘internalization and binding without blocking’ and ‘binding’ assays of the type described in Kelly et al. (2001) and/or by using one or more of the assays described in the examples herein, such as the assays described in Example 1D, 2D, 3D and 5B. In these ‘internalization and binding with blocking’ assays, cells may be blocked by the addition of 1 mg/mL ssDNA, for example as described in Kelly et al. (2001).

Alternatively or in addition to these ‘internalization and binding with blocking’, ‘internalization and binding without blocking’ and ‘binding’ assays and/or the assays described in the examples herein, antibodies may be used to correlate hTfR1 expression with aptamer cell staining, as also described in Kelly et al. (2001). For these antibody experiments, unlabelled anti-hTfR antibody may, for example, be purchased from BD systems (Cat #555534; San Jose CA).

Alternatively or in addition to these ‘internalization and binding with blocking’, ‘internalization and binding without blocking’ and ‘binding’ assays and/or the assays described in the examples herein, and/or the use of anti-hTfR antibodies, the specificity of aptamer binding to hTfR1 may also be assessed by performing anti-hTfR siRNA knockdown experiments of the type described in Maier et al. (2016).

By “expressed natively” herein is meant that the TfR is transcribed and translated from the endogenous nuclear genome of the cells rather than transcribed and translated from a heterologous coding sequence introduced into the cells by recombinant DNA technology or from an endogenous coding sequence whose protein-coding sequence(s), locus/loci in the genome and/or regulatory sequences have been altered by recombinant DNA technology. The term “expressed natively” herein also excludes scenarios where the TfR is translated from an exogenously supplied mRNA.

### Certain Effects of Aptamer Binding

### Internalization

Preferably, the aptamer binds to hTfR1 on the surface of human cells and the compound comprising the aptamer is subsequently internalized by the human cells.

Some of the particularly advantageous aptamers disclosed herein are aptamers wherein the aptamer (i) binds to hTfR1 on the surface of human cells and the compound comprising the aptamer is subsequently internalized by the human cells; and also (ii) binds to transferrin receptor 1 (TfR1) on the surface of cells of a non-human primate (NHP) and the compound comprising the aptamer is subsequently internalized by the NHP cells. In some of these instances, the aptamer does not compete with Tf of the NHP for binding to the NHP TfR1.

The cells expressing hTfR1 or TfR1 on their surface may be brain capillary endothelial cells (BCECs) of the type that form the blood-brain barrier (BBB).

The cells expressing hTfR1 or TfR1 on their surface may be muscle cells.

The cells expressing hTfR1 or TfR1 on their surface may be skeletal muscle cells.

The cells expressing hTfR1 or TfR1 on their surface may be cardiac muscle cells.

The cells expressing hTfR1 or TfR1 on their surface may be cancer cells.

In any of these instances of aptamer binding and internalization by NHP cells, the NHP may be cynomolgus monkey.

### Inhibition of Viral Infection

The aptamers disclosed herein may be capable of inhibiting infection of human osteocarcinoma cells by recombinant New World hemorrhagic fever mammarenaviruses (NWM) in culture.

The compounds and aptamer conjugates disclosed herein may benefit from the same advantageous property, and therefore the statements regarding inhibiting infection below apply equally to the aptamers, compounds, and aptamer conjugates disclosed herein.

The aptamers disclosed herein may be capable of inhibiting infection of human osteocarcinoma cells by recombinant NWM in culture with an EC50 of less than 750 nmol/l. The aptamers disclosed herein may be capable of inhibiting infection of human osteocarcinoma cells by recombinant NWM in culture with an EC50 of less than 500 nmol/l. The aptamers disclosed herein may be capable of inhibiting infection of human osteocarcinoma cells by recombinant NWM in culture with an EC50 of less than 250 nmol/l. The aptamers disclosed herein may be capable of inhibiting infection of human osteocarcinoma cells by recombinant NWM in culture with an EC50 of less than 100 nmol/l. The aptamers disclosed herein may be capable of inhibiting infection of human osteocarcinoma cells by recombinant NWM in culture with an EC50 of less than 50 nmol/l.

The potency of inhibition of NWM infection by the aptamers disclosed herein may be determined using human U-2 OS osteocarcinoma cells and recombinant vesicular stomatitis viruses (rVSVs) modified to express the glycoproteins from Junin virus (JUNV) or Machupo virus (MACV) in place of the VSV glycoprotein G, using as a control an rVSV expressing the glycoprotein from Lassa virus (LASV). A suitable assay to determine the potency of aptamers capable of inhibiting infection of human osteocarcinoma cells by recombinant NWM is described in Kelly et al. (2021).

### Tumour Staining

The detectably-labelled aptamer conjugates disclosed herein may demonstrate significant tumor staining in an in vivo cancer xenograft model. For example, the detectably-labelled aptamer conjugates disclosed herein may demonstrate significant tumor staining in an in vivo prostate cancer xenograft model. Suitable prostate cancer xenograft models include the in vivo 22Rv1 prostate cancer xenograft model described in Kelly et al. (2021) and in Example 15 herein.

In some embodiments, the aptamers disclosed herein would, when viewed 12 h post injection in the 22Rv1 model, both dorsally and laterally, demonstrate significant tumor staining.

In some embodiments, the aptamers disclosed herein would, when viewed 12 h post injection in the PC3-PSMA tumor model, both dorsally and laterally, demonstrate significant tumor staining.

In some embodiments, the aptamers disclosed herein would, when viewed 12 h post injection in the 22Rv1 model, both dorsally and laterally, demonstrate equivalent or increased tumor staining relative to a known hTfR aptamer (such as the Waz, C2 or C2.min aptamer).

In some embodiments, the aptamers disclosed herein would, when viewed 12 h post injection in the PC3-PSMA tumor model, both dorsally and laterally, demonstrate equivalent or increased tumor staining relative to a known hTfR aptamer (such as the Waz, C2 or C2.min aptamer).

In some instances, the detectably-labelled aptamer conjugates disclosed herein demonstrate significant tumor staining in an A431 in vivo cancer xenograft model, as described in Example 16 herein. In some instances, the detectably-labelled aptamer conjugates disclosed herein demonstrate increased tumor staining relative to a known hTfR aptamer (such as the Waz, C2 or C2.min aptamer) when viewed 24 h post injection in the A431 model.

In some instances, the detectably-labelled aptamer conjugate comprises the SEQ ID NO: 43 consensus sequence and demonstrates increased tumor staining relative to a known hTfR aptamer (such as the Waz, C2 or C2.min aptamer) when viewed 24 h post injection in the A431 model. In some instances, the aptamer comprises the SEQ ID NO: 38 consensus sequence and demonstrates increased tumor staining relative to a known hTfR aptamer (such as the Waz, C2 or C2.min aptamer) when viewed 24 h post injection in the A431 model.

In some instances, the detectably-labelled aptamer conjugate comprises SEQ ID NO: 45 demonstrates increased tumor staining relative to a known hTfR aptamer (such as the Waz, C2 or C2.min aptamer) when viewed 24 h post injection in the A431 model. In some instances, the aptamer comprises SEQ ID NO: 41 demonstrates increased tumor staining relative to a known hTfR aptamer (such as the Waz, C2 or C2.min aptamer) when viewed 24 h post injection in the A431 model.

### Certain Combinations of Aptamer Properties

Some aptamers disclosed herein are advantageous because they bind to hTfR1 without competing with hTf for binding to hTfR1, and because they benefit from improved in vivo stability relative to known hTfR1 aptamers in light of the fact that all of the sugar moieties of the nucleosides in the aptamer are chemically modified.

Some of the aptamers disclosed herein are especially advantageous because they bind to hTfR1 without competing with hTf for binding to hTfR1 and benefit from improved in vivo stability relative to known hTfR1 aptamers in light of the fact that all of the sugar moieties of the nucleosides in the aptamer are chemically modified, and additionally (1) are shorter than known hTfR1 aptamers, so can be synthesised more efficiently.

Some of the aptamers disclosed herein are especially advantageous because they bind to hTfR1 without competing with hTf for binding to hTfR1 and benefit from improved in vivo stability relative to known hTfR1 aptamers in light of the fact that all of the sugar moieties of the nucleosides in the aptamer are chemically modified, and additionally (2) bind also to non-human primate (NHP) transferrin receptor 1 (TfR1), in light of the fact that they bind an epitope on hTfR1 that is shared between hTfR1 and NHP TfR1.

Some of the aptamers disclosed herein are especially advantageous because they bind to hTfR1 without competing with hTf for binding to hTfR1 and benefit from improved in vivo stability relative to known hTfR1 aptamers in light of the fact that all of the sugar moieties of the nucleosides in the aptamer are chemically modified, and additionally (3) have improved hTfR1 binding affinity relative to known hTfR1 aptamers.

Some of the aptamers disclosed herein are especially advantageous because they bind to hTfR1 without competing with hTf for binding to hTfR1 and benefit from improved in vivo stability relative to known hTfR1 aptamers in light of the fact that all of the sugar moieties of the nucleosides in the aptamer are chemically modified, and additionally (1) are shorter than known hTfR aptamers, so can be synthesized more efficiently, and (2) bind also to non-human primate (NHP) transferrin receptor 1 (TfR1), in light of the fact that they bind an epitope on hTfR1 that is shared between hTfR1 and NHP TfR1.

Some of the aptamers disclosed herein are especially advantageous because they bind to hTfR1 without competing with hTf for binding to hTfR1 and benefit from improved in vivo stability relative to known hTfR1 aptamers in light of the fact that all of the sugar moieties of the nucleosides in the aptamer are chemically modified, and additionally (1) are shorter than known hTfR aptamers, so can be synthesized more efficiently, and (3) have improved hTfR1 binding affinity relative to known hTfR1 aptamers.

Some of the aptamers disclosed herein are especially advantageous because they bind to hTfR1 without competing with hTf for binding to hTfR1 and benefit from improved in vivo stability relative to known hTfR1 aptamers in light of the fact that all of the sugar moieties of the nucleosides in the aptamer are chemically modified, and additionally (2) bind also to non-human primate (NHP) transferrin receptor 1 (TfR1), in light of the fact that they bind an epitope on hTfR1 that is shared between hTfR1 and NHP TfR1, and (3) have improved hTfR1 binding affinity relative to known hTfR1 aptamers.

Some of the aptamers disclosed herein are especially advantageous because they bind to hTfR1 without competing with hTf for binding to hTfR1 and benefit from improved in vivo stability relative to known hTfR1 aptamers in light of the fact that all of the sugar moieties of the nucleosides in the aptamer are chemically modified, and additionally (1) are shorter than known hTfR aptamers, so can be synthesized more efficiently, and (2) bind also to non-human primate (NHP) transferrin receptor 1 (TfR1), in light of the fact that they bind an epitope on hTfR1 that is shared between hTfR1 and NHP TfR1, and (3) have improved hTfR1 binding affinity relative to known hTfR1 aptamers.

### Aptamer Conjugates

The aptamers disclosed herein may be conjugated to at least one other molecular entity to form an aptamer conjugate. In some instances, the compounds disclosed herein are an aptamer conjugate in which an aptamer disclosed herein is conjugated to one or more of a detectable label, an antisense compound, a small organic molecule of less than 2000 Daltons (Da), polyethylene glycol (PEG), a nanoparticle, or a combination thereof.

In some embodiments, the aptamer conjugate comprises any of the aptamer disclosed herein and other molecular entity. In some embodiments, the other molecular entity is conjugated to the 5′ end of the aptamer. In some embodiments, the other molecular entity is conjugated to the 3′ end of the aptamer. In some embodiments, the aptamer conjugate comprises a linker between the aptamer and the other molecular entity. In some embodiments, the aptamer and the other molecular entity are conjugated directly without a linker.

In some embodiments, the aptamer conjugate comprises a core sequence of the aptamer provided herein without the 5′ stem region or 3′ stem region. In some embodiments, the aptamer conjugate comprises one or more core sequences of the aptamers provided herein. In some embodiments, the aptamer conjugate comprises more than one core sequence of the aptamers provided herein.

In some embodiments, the aptamer conjugate comprises an aptamer and PEG, cholesterol, oligonucleotide (e.g., siRNA, anti-sense oligonucleotide, miRNA), or a combination thereof. In some embodiments, the aptamer conjugate comprises an aptamer and a protein (e.g., enzyme, antibody, etc.).

In some embodiments, the aptamer conjugate comprises an aptamer and a liposome. In some embodiments, the aptamer conjugate comprises an aptamer and a lipid nanoparticle. In some embodiments, the liposome or the lipid nanoparticle contains a therapeutic compound. In some embodiments, the aptamer is conjugated to a vehicle for delivery of a therapeutic compound. In some embodiments, the aptamer conjugate comprises an aptamer and a therapeutic compound. In some embodiments, the aptamer is conjugated to a gene therapy product. In some embodiments, the aptamer is conjugated to a virus. In some embodiments the aptamer is conjugated to the recombinant AAV (rAAV) or a lentivirus. In some embodiments, the aptamer is conjugated to a small molecule drug.

In some embodiments, the aptamer conjugate comprises an aptamer and a dye.

In some embodiments, the aptamer conjugate comprises more than one aptamer. In some embodiments, the aptamer conjugate comprises two or more different aptamers. In some embodiments, the aptamer conjugate comprises two or more of the same aptamers. In some embodiments, the aptamer conjugate comprises two or more different aptamers binding to different targets. In some embodiments, the aptamer conjugate comprises two or more different aptamers binding to the same target.

In some embodiments, one aptamer is conjugated to more than one molecular entity.

In one aspect, the present disclosure provides a lipid nanoparticle decorated with one or more aptamers disclosed herein. In some embodiments, the lipid nanoparticle is decorated with a single type of aptamer. In some embodiments, the lipid nanoparticle is decorated with more than one type of aptamer.

### Linker

The aptamers disclosed herein may be conjugated to a molecular entity using any suitable method known in the art. For example, the aptamers disclosed herein may be conjugated to a molecular entity using the methods described in Nerantzaki et al., Polym Chem (2021), 12, 3498-3509. A molecular entity may be conjugated to the 5′ end and/or the 3′ end of the aptamer.

The aptamers disclosed herein may be conjugated directly or indirectly to the molecular entity. For example, a molecular entity may be conjugated to the aptamer using a hexaethylene glycol linker. Any of the linkers known in the art can be used in various embodiments. As used herein, the term “linker” means a moiety that connects two parts of a compound. Linkers typically comprise a direct bond or an atom such as oxygen or sulfur, a unit such as NH, C(O), C(O)O, C(O)NH, S, SS, SO, SO2, SO2NH, P(O)(O—), or a chain of atoms. In some embodiments, the aptamer and the molecular entity are linked by a phosphodiester or a modified intersugar linkage described herein.

The molecular entity may be conjugated to the aptamer using a DNA linker, optionally wherein the DNA linker is a three-nucleoside linker having the sequence deoxythymine (dT), 5-methyl deoxycytidine (5mdC), deoxyadenine (dA), The molecular entity may be conjugated to the aptamer using a hexylamine linker (C6NH₂). In some embodiments, the aptamer is conjugated to a molecular entity using a hexa-ethyleneglycol spacer (Sp18) linker. In some embodiments, the NDA linker comprises C6Spacer Amidite (DMT-1,6-Hexandiol) or C12 Spacer Amidite (DMT-1,12-dodecanediol). In some embodiments, the linker is a hexaethylene glycol linker (e.g., a hexaethylene glycol spacer linker (Sp18)), a C6 linker (e.g., comprising C6 Spacer Amidite (DMT-1,6-Hexandiol)), a C12 linker (e.g., comprising C12 Spacer Amidite (DMT-1,12-dodecanediol)), a T linker, or a TTT linker.

In some embodiments, the linker between the aptamer and the molecular entity is a nucleotidic linker. As used herein, a “nucleotidic linker” refers to an oligonucleotide that connects an aptamer to an oligonucleotide agent. Without limitations, the nucleotidic linker can be single-stranded or a double-stranded oligonucleotide, e.g., a linker comprising a first oligonucleotide strand and second oligonucleotide strand, wherein the first and the second strands are sufficiently complementary to each other. Furthermore, the nucleotidic linker can comprise one or more of the oligonucleotide modifications described herein. In some embodiments, the nucleotidic linker is linked to the aptamer and the molecular entity by a phosphodiester linkage or by an intersugar linkage modification described herein.

For a single-stranded nucleotidic linker, the aptamer can be at the 5′-end or the 3′-end of the linker. For a double-stranded nucleotidic linker, the aptamer and the molecular entity can be linked to different strands of the linker. Furthermore, the aptamer and the molecular entity can be at the 5′-end or the 3′-end of the strand they are linked to. In some embodiments, both the aptamer and the molecular entity are at the 5′-end of the strand to which they are linked. In some other embodiments, both the aptamer and the molecular entity are at the 5′-end of the strand to which they are linked. In yet some other embodiments, one of the aptamer and the molecular entity is at the 5′-end and the other is at the 3′-end of the strand to which they are which they are linked.

The nucleotidic linker can itself be linked by a non-nucleotidic linker to the oligonucleotide agent and/or the aptamer. Without limitations, a non-nucleotidic linker can be linked to the aptamer, the molecular entity and/or a nucleotidic linker by a phosphodiester linkage or an oligonucleotide intersugar modification described herein.

In some embodiments, the nucleotidic linker is linked to the molecular entity by a non-nucleotidic linker, and the nucleotidic linker is not linked to the aptamer by a non-nucleotidic linker. In some further embodiments of this, the nucleotidic linker is linked to the molecular entity by a non-nucleotidic linker, and the nucleotidic linker is linked to the aptamer by a phosphodiester linkage or an oligonucleotide intersugar modification described herein.

A nucleotidic linker can be of any length, e.g., between 4-30 nucleotides in length. A double-stranded linker can comprise between 5-30 nucleotide basepairs, in some embodiments, 5-20 or 4-20, 5-15, or 4-15 bp linker is used. Accordingly, in some embodiments the double-stranded linker comprises 5, 6, 7, 8, 9, 10, 11, 12, 13, 14, 15, 16, 17, 18, 19, or 20 nucleotide base pairs.

In some embodiments, the conjugate comprises a non-nucleotidic linker. In some embodiments, the non-nucleotidic linker comprises at least one cleavable linking group, i.e. the linker is a cleavable linker. As used herein, a “cleavable linker” refers to linkers that are capable of cleavage under various conditions. Conditions suitable for cleavage can include, but are not limited to, pH, UV irradiation, enzymatic activity, temperature, hydrolysis, elimination and substitution reactions, redox reactions, and thermodynamic properties of the linkage. In some embodiments, a cleavable linker can be used to release the linked components after transport to the desired target. The intended nature of the conjugation or coupling interaction, or the desired biological effect, will determine the choice of linker group.

### Aptamer-Ligand Conjugates

The aptamers disclosed herein may be conjugated to a ligand such as polyethylene glycol (PEG) or cholesterol. A ligand that can increase circulation time of the aptamer by reducing the rate of renal clearance can be used in various embodiments.

The ligand can be coupled to the aptamer or the aptamer conjugates described herein. When a ligand is added to an aptamer conjugate comprising an aptamer and a payload, the ligand can be coupled to the aptamer and/or the payload. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEGI2, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, prolamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g., steroids, bile acids, cholesterol, cholic acid, adamantane acetic acid, 1-pyrene butyric acid, dihydrotestosterone, 1,3-Bis-0(hexadecyl)glycerol, geranyloxyhexyl group, hexadecylglycerol, borneol, menthol, 1,3-propanediol, heptadecyl group, palmitic acid, myristic acid, 03-(oleoyl) lithocholic acid, 03-(oleoyl) cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-KB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a-helical cell-permeation agent).

In some embodiments, the aptamer conjugate comprises a PEG having a molecular weight of at least about 5 kDa, at least about 10 kDa, at least about 20 kDa, or at least about 40 kDa. In some instances, the aptamer conjugate comprises a PEG having a molecular weight of about 20 kDa PEG. The PEG modification may be conjugated to the aptamer via a hexylamine linker (C₆NH₂).

In some embodiments, the aptamer conjugate comprises a cholesterol, tocopherol, docosanoic acid (DCA), docosahexaenoic acid, or dicarboxylic acids (e.g., with 8-24 carbons).

In some embodiments, the aptamer conjugate comprises the unsaturated fatty acids docosahexaenoic acid (DHA, 22:6 n-3) and eicosapentaenoic acid (EPA, 20:5 n-3); the saturated fatty acid docosanoic acid (DCA, 22:0); the sterols cholesterol (Chol) and lithocholic acid (LA); and the vitamins retinoic acid (RA) or α-tocopheryl succinate (TS). In some embodiments, the aptamer conjugate comprises alkyl lipids, e.g., C16 (palmitate).

In some embodiments, the aptamer conjugate comprises a ligand on the 5′ end or 3′ end of the aptamer.

### Aptamer-Payload Conjugates

One aspect of the present disclosure provides a conjugate comprising an aptamer and a payload. The payload can be a therapeutic molecule. The payload can be a vehicle for delivering therapeutic molecules. The aptamer-payload conjugate can further comprise a ligand and/or a linker.

In some embodiments, the payload is conjugated to the 5′ side of the aptamer. In some embodiments, the payload is conjugated to the 3′ side of the aptamer. In some embodiments, the payload is directly conjugated to the aptamer without a linker.

In some embodiments, the aptamer conjugate comprises a payload, a linker, and an aptamer from 5′ to 3′ direction. In some embodiments, the aptamer conjugate comprises an aptamer, a linker and a payload from 5′ to 3′ direction. In some embodiments, the aptamer conjugate further comprises a ligand at 5′ or 3′ end. In some embodiments, the linker is a hexa-ethyleneglycol spacer (Sp18) linker. In some embodiments, the linker comprises PEG.

In some embodiments, the aptamer conjugate further comprises a ligand conjugated to 5′ or 3′ end. In some embodiments, the aptamer conjugate comprises a payload, a linker, an aptamer and a ligand from 5′ to 3′ direction. In some embodiments, the aptamer conjugate comprises a ligand, a payload, a linker and an aptamer from 5′ to 3′ direction. In some embodiments, the aptamer conjugate comprises a ligand, an aptamer, a linker and a payload from 5′ to 3′ direction. In some embodiments, the aptamer conjugate comprises an aptamer, a linker, a payload and a ligand from 5′ to 3′ direction. In some embodiments, the ligand comprises polyethylene glycol (PEG, e.g., PEG-2K, PEG-5K, PEG-10K, PEG-12K, PEG-15K, PEG-20K, PEG-40K).

In some embodiments, the payload is an oligonucleotide drug. In some embodiments, the payload is an siRNA and the conjugate is an aptamer-siRNA conjugate. In some embodiments, the payload is an antisense oligonucleotide (ASO) to form an aptamer-ASO conjugate. In some embodiments, the payload is double stranded oligonucleotide and the aptamer is conjugated to a sense strand of the oligonucleotide drug. In some embodiments, the payload is double stranded oligonucleotide and the aptamer is conjugated to an anti-sense strand of the oligonucleotide drug.

In some instances, the ASO is conjugated to the aptamer using a hexaethylene glycol linker. In some instances, the ASO is conjugated to the aptamer by hybridization.

In some embodiments, the payload is protein therapeutics. In some embodiments, the payload is an antibody or a variant thereof.

In some embodiments, the payload is a small molecule drug.

In some embodiments, the payload is a nanoparticle to form an aptamer-nanoparticle conjugate. As used herein, the term “nanoparticle” refers to a particle of matter that is 1 to 1000 nanometres (nm) in diameter.

The aptamers disclosed herein may be conjugated to a lipid nanoparticle (LNP) to form an aptamer-LNP conjugate. As used herein, “lipid nanoparticle” or “LNP” refers to a nanoparticle that comprises one or more lipid components. The term “lipid” refers to a class of organic compounds that are characterized by being insoluble in water but soluble in many organic solvents. In some instances, the LNP is composed of one or more lipid bilayers that enclose an aqueous solution core.

In some instances, the LNP comprises an ionizable cationic lipid. Ionizable cationic lipids typically comprise a tertiary amine that is deprotonated at neutral (or near neutral pH) and is positively charged in pH conditions below the acid-dissociation constant (pKₐ) of the lipid.

In some instances, the ionizable cationic lipid is SM-102 (heptadecan-9-yl 8-((2-hydroxyethyl)(6-oxo-6-(undecyloxy)hexyl)amino)octanoate).

In some instances, the LNP comprises a helper lipid. Helper lipids support stability of the LNP during storage and circulation and may be neutral lipids, anionic lipids or zwitterionic lipids. For example, a helper lipid may be a phospholipid or a glycerolipid. In some instances, the helper lipid is DSPC (1,2-distearoyl-sn-glycero-3-phosphocholine).

In some instances, the LNP comprises a sterol lipid, such as cholesterol and its derivatives. In some instances, the sterol lipid is cholesterol. Cholesterol can regulate the fluidity of an LNP membrane. In some instances, the LNP comprises a sterol lipid in addition to a helper lipid.

In some instances, the LNP comprises a PEG-modified lipid. PEG-modified lipids control LNP size and stability. In some instances, the PEG-modified lipid is DMG-PEG2000 (1,2-dimyristoyl-rac-glycero-3-methoxypolyethylene glycol-2000).

In some instances, the LNP comprises an ionizable cationic lipid, a helper lipid, a sterol lipid (e.g., cholesterol) and a PEG-modified lipid.

In some such instances, the LNP comprises SM-102, DSPC, cholesterol and DMG-PEG2000.

The aptamer-LNP conjugates can be used to deliver an LNP to a target cell in vivo or in vitro. The LNP typically comprises a molecular cargo. The molecular cargo may be attached to the surface of the LNP or encapsulated within the LNP. In some instances, the molecular cargo is a therapeutic or diagnostic agent. In some instances, the molecular cargo is a nucleic acid. The nucleic acid may be, for example, a single-stranded antisense oligonucleotide, an siRNA or an mRNA.

The aptamer can be conjugated to the LNP using any suitable method known in the art. For example, the aptamer may be conjugated to a lipid using a 5′ C6 terminal thiol.

In some instances, the aptamer-LNP conjugate comprises an aptamer-lipid conjugate. The aptamer-lipid conjugate may be an aptamer-DSPE (1,2-distearoyl-sn-glycero-3-phosphoethanolamine) conjugate. In some instances, the aptamer-LNP conjugate is produced by insertion of an aptamer-lipid conjugate into a pre-formed LNP.

### Aptamer-Dye Conjugates

In the aptamer conjugates disclosed herein, the aptamer may be conjugated to a detectable label. The detectable label may be a fluorescent dye molecule, such as a fluorescent dye molecule selected from Alexa Fluor 488 (AF488), Fluorescein, Cy3, Cy5, Alexa Fluor 647 (AF647), DyLight 650 (DY650), BP Fluor 647 (BP647), Alexa Fluor 750 (AF750), and IRDye® 800CW. The detectable label may be conjugated to the 5′ end of the aptamer via a 5′ thioester linkage or the 5′ end of the aptamer by a 5′ amide linkage. The detectable label may be conjugated to the 3′ end of the aptamer via a 3′ thioester linkage or the 3′ end of the aptamer by a 3′ amide linkage.

Suitable methods of aptamer dye conjugation are known in the art and described, for example, in Maier et al. (2016) and Kelly et al. (2021) and in Example 17 herein.

### Compositions

Disclosed herein is a composition comprising any of the compounds disclosed herein and one or more carriers or diluents.

Also disclosed herein is a composition comprising a plurality of different compounds as disclosed herein and one or more carriers or diluents.

The composition can be a pharmaceutical composition or a composition for diagnostic use. In another aspect, the present disclosure provides a method of using the aptamer or the aptamer conjugate for biomedical application. In some embodiments the aptamer or the aptamer conjugate is used for targeted drug delivery, diagnosis, or imaging.

### Pharmaceutical Compositions

Disclosed herein is a pharmaceutical composition comprising any of the compounds disclosed herein and one or more pharmaceutically acceptable carriers or diluents. Also disclosed herein is a pharmaceutical composition comprising a plurality of different compounds as disclosed herein and one or more pharmaceutically acceptable carriers or diluents.

The pharmaceutical compositions disclosed herein are suitable for use in a mammalian subject, for example for use in therapy or diagnosis.

Appropriate pharmaceutically acceptable carriers or diluents are known in the art. Pharmaceutical compositions as disclosed herein can be formulated and tested as appropriate for their intended use, following principles and guidance known in the art (for example, as described in Remington: The Science and Practice of Pharmacy, Twenty-third Edition 2020).

### Methods and Uses of the Aptamers, Aptamer Conjugates and Compositions

Also disclosed herein is a pharmaceutical composition as disclosed herein, for use in treating a disease or condition in a mammalian subject in need thereof, the use comprising administering a therapeutically effective amount of the pharmaceutical composition to the subject in need thereof and thereby treating the disease or condition.

Also disclosed herein is a pharmaceutical composition as disclosed herein, for use in diagnosing a disease or condition in a mammalian subject in need thereof, the use comprising administering a diagnostically effective amount of the pharmaceutical composition to the subject in need thereof and thereby treating the disease or condition.

Also disclosed herein is the use of a pharmaceutical composition as disclosed herein in the manufacture of a medicament, for use in treating a disease or condition in a mammalian subject in need thereof.

Also disclosed herein is the use of a pharmaceutical composition as disclosed herein in the manufacture of a medicament, for use in diagnosing a disease or condition in a mammalian subject in need thereof.

Also disclosed herein is a method of treating a disease or condition in a mammalian subject in need thereof, the method comprising administering a therapeutically effective amount of pharmaceutical composition disclosed herein to the subject in need thereof and thereby treating the disease or condition.

Also disclosed herein is a method of diagnosing a disease or condition in a mammalian subject in need thereof, the method comprising administering a diagnostically effective amount of pharmaceutical composition disclosed herein to the subject in need thereof and thereby diagnosing the disease or condition.

By “treating a disease or condition” herein (and equivalent uses of the term “treating” or “treat”) is meant ameliorating at least one symptom of the disease or condition.

By “diagnosing a disease or condition” herein (and equivalent uses of the term “diagnosis” or “diagnosis”) is meant identifying the presence of the disease or condition in the subject. The diagnostic uses disclosed herein may involve in vitro and/or in vivo steps.

Diseases and conditions that are susceptible to treatment or diagnosis using the using the aptamers and aptamer conjugates described herein can readily be identified by the skilled person, in light of what is already known in the art of the relevance of hTfR1 and the targeting of cell-surface hTfR1 in therapy and diagnosis.

In any of these therapeutic and diagnostic uses, the disease or condition may be a disease or condition of the brain.

In any of these therapeutic and diagnostic uses, the disease or condition may be a disease or condition of the muscle.

In any of these therapeutic and diagnostic uses, the disease or condition may be cancer.

Methods of treating a disease or condition in a mammalian subject in need thereof can be designed and tested as appropriate for the disease or condition of interest, following principles and guidance known in the art (for example, as described in Remington: The Science and Practice of Pharmacy, Twenty-third Edition 2020). Suitable dosages and administration protocols can readily be designed and tested by the skilled person based on what is known in the art.

### General

As used herein, references to the singular forms “a”, “an” and “the” include reference to their plural equivalents, unless the context clearly dictates otherwise. Thus, for example, references herein to “an aptamer” are references to one or more such aptamers and references to “a cell” are references to one or more such cells.

The term “comprising” encompasses “including” as well as “consisting of”, e.g. a composition “comprising” X may consist exclusively of X or may include something additional, e.g. X+Y.

As used herein, the term “known hTfR1 aptamers” can refer to any aptamer known in the art to bind to hTfR1. It may for example refer to the Waz aptamer, the C2 aptamer and/or the C2.min aptamer.

The nucleoside sequence of the Waz aptamer is 5′-GGGUUCUACGAUAAACGGUUAAUGACCAGCUUAUGGCUGGCAGUUCCC-3′ (SEQ ID NO: 60). Waz comprises 2′-fluoro-modified pyrimidine nucleosides and unmodified (2′-hydroxyl) purine nucleosides.

The nucleoside sequence of the C2 aptamer is 5′-UCAAACAUCUCACAGAUCAAUCCAAGGGACCUCGUUAAAGGACGACUCCC-3′ (SEQ ID NO: 65). C2 comprises 2′-fluoro-modified pyrimidine nucleosides and unmodified (2′-hydroxyl) purine nucleosides.

The nucleoside sequence of the C2.min aptamer is 5′-GGGGGAUCAAUCCAAGGGACCCGGAAACGCUCCCUUACACCCC-3′ (SEQ ID NO: 66). C2.min comprises 2′-fluoro-modified pyrimidine nucleosides and unmodified (2′-hydroxyl) purine nucleosides.

In Examples 6, 10 and 11 herein, the inventors included the “AP-00074” hTfR1 aptamer (SEQ ID NO: 55) as a comparator. This comparator aptamer corresponds to Waz with a single point mutation and similarly comprises 2′-fluoro-modified pyrimidine nucleosides and unmodified (2′-hydroxyl) purine nucleosides. AP-00074 has equivalent hTfR1 binding characteristics to Waz.

Various instances of the invention are described herein. It will be recognised that the features specified for each instance may be combined with other specified features to provide further instances.

### BRIEF DESCRIPTION OF THE DRAWINGS

FIG. 1: Flow cytometry analysis of bead staining with anti-hTfR1 aptamers, as described in Example 1B. (A) Staining results for the aptamer libraries at Rounds 4 to 6 of the anti-hTfR1 aptamer selection described in Example 1A, in the presence or absence of hTf. (B) Staining results for c2.min assayed under the same conditions.

FIG. 2A and FIG. 2B: Flow cytometry analysis of cell staining with anti-hTfR1 aptamers, as described in Example 1B. FIG. 2A shows staining results for the aptamer libraries at Rounds 1, and 6 to 8 of the anti-hTfR1 aptamer selection described in Example 1A, in the presence or absence of hTf. FIG. 2B shows staining results for similar assays performed using c2.min and Waz.

FIG. 3: Flow cytometry analysis of bead staining with full-length anti-hTfR1 aptamer clones 36, 171, 1, 4, 27, 7310, 9350, 12687, and 3353, as well as c2.min, and Waz, as described in Example 1D, in the presence or absence of hTf. The grey peak denotes the results obtained with beads that were not incubated with aptamer library (beads only), the solid line denotes the results obtained with hTfR1 immobilized on beads in the absence of hTf, and the dotted line denotes the results obtained with hTfR1 immobilized on beads in the presence of hTf.

FIG. 4: Flow cytometry analysis of bead staining with truncated anti-hTfR1 aptamer clones bearing a 5′ ASO extension AP-00039 to AP-00047, as well as c2.min, and Waz, as described in Example 1D, in the presence or absence of hTf. The grey peak denotes the results obtained with beads that were not incubated with aptamer library (beads only), the solid line denotes the results obtained with hTfR1 immobilized on beads in the absence of hTf, and the dotted line denotes the results obtained with hTfR1 immobilized on beads in the presence of hTf.

FIG. 5: Flow cytometry analysis of cell staining with truncated anti-hTfR1 aptamer clones AP-00242 to AP-00249, as well as Waz, as described in Example 1D and Example 4A. The grey peak denotes the results obtained with cells only, and the solid line denotes the results obtained when aptamers were incubated with the cells.

FIG. 6A and FIG. 6B: Flow cytometry analysis of cell staining with anti-hTfR1 aptamers, as described in Example 2B. FIG. 6A shows staining results for the aptamer libraries at Rounds 2, 4, 6, and 9 of the anti-hTfR1 aptamer selection described in Example 2A, in the presence or absence of hTf. FIG. 6B shows staining results for c2.min assayed under the same conditions. The grey peak denotes the results obtained with cells only, and the solid line denotes the results obtained when aptamers were incubated with the cells.

FIG. 7: Flow cytometry analysis of cell staining with truncated anti-hTfR1 aptamer clones bearing an ASO extension AP-00039, AP-00257 to AP-00264, as well as Waz, as described in Example 2D and Example 4B, in the presence or absence of hTf. The grey peak denotes the results obtained with cells only, the solid line denotes the results obtained when aptamers were incubated with the cells in the absence of hTf, and the dotted line denotes the results obtained when aptamers were incubated with the cells in the presence of hTf.

FIG. 8: Flow cytometry analysis of bead staining with anti-hTfR1 aptamers, as described in Example 3B. Staining results are shown for the aptamer libraries at Rounds 7, 9, and 11 of the anti-hTfR1 aptamer selection described in Example 3A, in the presence or absence of hTf. The grey peak denotes the results obtained with beads that were not incubated with aptamer library (beads only), and the solid line denotes the results obtained when aptamers were incubated with hTfR1 immobilized on beads.

FIG. 9: Flow cytometry analysis of bead staining with truncated anti-hTfR1 aptamer clone bearing a 5′ ASO extension AP-00353, as well as Waz, as described in Example 3D, in the presence or absence of hTf. The grey peak denotes the results obtained with beads that were not incubated with aptamer library (beads only), the solid line denotes the results obtained when aptamers were incubated with the beads in the absence of hTf, and the dotted line denotes the results obtained when aptamers were incubated with the beads in the presence of hTf.

FIG. 10: Dose-dependent knockdown of HPRT1 mRNA compared to the housekeeping gene PPIA in HeLa cells by aptamers conjugated to an anti-HPRT1 siRNA AP-00283 to AP-00286, and AP-00517, as described in Example 6.

FIG. 11: Knockdown of HPRT1 mRNA compared to the housekeeping gene PPIB2 in calf muscle and cardiac tissue of hTfR1 knock-in mice treated with aptamers conjugated to an anti-HPRT1 siRNA AP-00284 to AP-00286 and their PEGylated versions, AP-307 to AP-309, as described in Example 7. *P<0.05, **P<0.005, ***P<0.0005.

FIG. 12: Knockdown of Malat-1 RNA compared to the housekeeping gene HPRT1 in calf muscle of hTfR1 knock-in mice treated with aptamers bearing a 5′ ASO extension AP-00340 to AP-00342 and their PEGylated versions, AP-350 to AP-352, as well as the ASO alone (AP-00593), as described in Example 8.

FIG. 13: Flow cytometry analysis of expression of EGFP in Hela cells incubated with LNPs functionalized with thiol-modified anti-hTfR1 aptamers AP-00436 (TfR-Apt-LNP) or AP-00438, LNPs functionalized with thiol-modified non-functional control aptamer AP-00435 (Cntl-Apt-LNP), and LNPs bearing BME-quenched DSPE-PEG (2000)-maleimide (BME-LNP). The grey peak denotes cells which were not incubated with LNPs.

FIG. 14: Flow cytometry analysis of expression of EGFP in Hela cells incubated with LNPs, BME-LNPs, Cntl-Apt-LNPs, or TfR-Apt-LNPs. Each experiment was performed with the LNP/LNP-conjugate alone (in buffer only), the LNP/LNP-conjugate in the presence of 5 μM Cntl-Apt (AP-000428) or the LNP/LNP-conjugate in the presence of 5 μM TfR-Apt (AP-000385).

FIG. 15: Flow cytometry analysis of bead staining with truncated anti-hTfR1 aptamer clones bearing a 5′ C₆ terminal amine (C₆NH₂) AP-00074, AP-00248, and AP-00385, and truncated anti-hTfR1 aptamer clone bearing a 5′ ASO extension AP-00264, as well as Waz, whereby either hTfR1 or cTfR1 was immobilized on the beads, as described in Example 10. The grey peak denotes the results obtained with beads that were not incubated with aptamer library (beads only), and the solid line denotes the results obtained when aptamers were incubated with the beads.

FIG. 16: Fluorescent imaging data for mice injected with cells from an epidermal cancer cell line that expresses hTfR1. Following tumour growth, test animals were treated with fluorescently-labelled hTfR1-targeting aptamers or a fluorescently-labelled non-targeting control aptamer, as described in Example 16. *P<0.05, **P<0.005, ***P<0.0005.

FIG. 17: Computationally predicted secondary structures of the folded aptamer sequence of compound AP-248 (SEQ ID NO: 41) obtained using mfold software as described in Example 18.

FIG. 18: Computationally predicted secondary structures of the folded aptamer sequence of compound AP-264 (SEQ ID NO: 45) obtained using mfold software as described in Example 18.

FIG. 19: Computationally predicted secondary structures of the folded aptamer sequence of compound AP-385 (SEQ ID NO: 53) obtained using mfold software as described in Example 18.

FIG. 20: Expression of DMPK in tissues from hTfR1 knock-in mice treated with ASO only (AA-05275), aptamer-ASO conjugates targeting hTfR1 (AP-00548; AP-00550), or aptamer-ASO conjugates bearing a non-functional ASO (TA-00615; TA-00616) was measured by qPCR. Animals were administered the dose as indicated on Days 1, 5, and 9. Animals were euthanized and tissues extracted on Day 14 following administration of the initial dose.

FIG. 21: Tissues harvested from hTfR1 knock-in mice treated with ASO only (AA-05275), aptamer-ASO conjugates targeting hTfR1 (AP-00548; AP-00550), or aptamer-ASO conjugates bearing a non-functional ASO (TA-00615; TA-00616) were evaluated by hybridization ELISA for ASO concentrations. Animals were administered the dose as indicated on Days 1, 5, and 9. Animals were euthanized and tissues extracted on Day 14 following administration of the initial dose.

FIG. 22: Results show DMPK expression in calf, diaphragm, and heart tissue harvested from hTf1R knock-in mice treated with aptamer-ASO conjugates as indicated (see Table 24). Animals were administered 3 mg/kg of the indicated conjugate on Days 1, 5, and 9. Animals were euthanized and tissues extracted on Day 16. Expression of DMPK RNA was measured by qPCR.

FIG. 23: Results show DMPK expression in tissue harvested from hTf1R knock-in mice treated with select compounds as indicated. Animals were administered 0, 0.3, 1, 3, 10, or 30 mg/kg of compound on Days 1, 5, and 9. Animals were euthanized and tissues extracted on Day 16. Expression of DMPK RNA was measured by qPCR.

FIG. 24: Results show DMPK expression in tissue harvested from hTf1R knock-in mice treated with select aptamer-ASO conjugate compounds (TA-00598, TA-00601, TA-00602, TA-00603, TA-00604), a lipidated ASO only compound (AA-05385), or

PBS. Animals were administered 3 mg/kg of the indicated compound by either intravenous (iv) or subcutaneous (sc) administration on Days 1, 5, and 9. Animals were euthanized and tissues extracted on Days 16, 23, or 30, corresponding to 7, 14, and 21 days post last dose. Expression of DMPK RNA was measured by qPCR.

FIG. 25: Results of a flow cytometry-based competition assay to assess binding of select compounds in Jurkat cells. Unlabeled compounds were premixed with AF647-labeled AP-00385 in 10-fold molar excess. Mixtures were added to Jurkat cells and supplemented with ssDNA as a blocking agent and 1 mg/mL Transferrin to investigate competition binding in the presence of the natural ligand. Cells were incubated, washed, and then assayed by flow cytometry.

FIG. 26: Results of a competition assay to assess binding of select compounds in Jurkat cells. Unlabeled compounds were assessed at a range of concentrations (0.25 μM, 0.5 μM, 1 μM, 2 μM), each premixed with 400 nM AF647-labeled AP-00385. Mixtures were added to Jurkat cells and supplemented with ssDNA as a blocking agent and 1 mg/mL Transferrin to investigate competition binding in the presence of the natural ligand. Cells were incubated, washed, and then assayed by flow cytometry.

### EXAMPLES

The following examples are given for the purpose of illustrating various instances of the invention and are not meant to limit the present invention in any fashion. These examples, along with the methods described, are representative of preferred instances and are not intended as limitations on the scope of the invention.

### Example 1

A. Selection for Nuclease Stabilized (fGmH) Anti-hTfR1 Aptamers

Anti-hTfR1 aptamers were identified using an N45 library comprised of a 45-nucleotide random region flanked by constant regions. The library sequence and the sequences of oligos used to amplify the library are described in Table 1. For nuclease stability, the library was composed of a mixture of 2′-fluoro-guanosine (G-2′F), 2′-O-methyl adenosine (A-2′OMe), 2′-O-methyl cytosine (C-2′OMe) and 2′-O-methyl uridine (U-2′OMe). Oligonucleotides comprising this combination of modified nucleosides are denoted “fGmH”.

TABLE 1
Library sequence and sequences of oligos
used to amplify the library

| | Sequence (5′ to 3′) |
|---|---|
| Library sequence | GGGAGAATGCCGTAGCAGCGAGAATAG- |
| (Total library | T-N45-T-TATACATGGCTTGAGCCGGC |
| length: 94 bases) | (SEQ ID NO: 1) |
| N45.F | GGGAGAATGCCGTAGCAGCGAGAATAG |
| | (SEQ ID NO: 2) |
| N45.R | GCCGGCTCAAGCCATGTATA (SEQ ID NO: 3) |

Where G, A, T and C are deoxyribonucleotides; “N45” is a 45-nucleotide random region; the N45-F primeralso includes a 5′ phage polymerase promoter.

The starting library was transcribed from a pool of ~10¹⁴ double-stranded DNA (dsDNA) molecules. The dsDNA library was generated by primer extension using Klenow exo (−) DNA polymerase, the pool forward primer (N45.F (SEQ ID NO: 2)) and a synthetic single-stranded DNA (ssDNA) molecule encoding the reverse complement of the library. The dsDNA was subsequently converted to 100% backbone modified RNA via transcription using a mixture of 2′F GTP, 2′OMe ATP/CTP/UTP and a modified phage polymerase in buffer optimized to facilitate efficient transcription. Following transcription, RNAs were treated with DNAse to remove the template dsDNA and purified.

The selection strategies used to isolate the anti-hTfR1 aptamers described herein were designed to drive the selection for aptamers that bind to the surfaces of the receptor that are distinct from the binding site of the natural ligand, human transferrin (hTf). Aptamers that bind hTfR1 that do not compete with hTf are desirable, as these will not compete with the natural ligand, hTf, which is present in serum at high (25 μM) concentration. Additionally, an anti-hTfR1 aptamer that binds to hTfR1 and does not compete with hTf may bind without blocking hTf and thereby altering iron metabolism of the cell.

For the first 6 rounds of the selection, human recombinant TfR bearing both an Avi-tag and a His-Tag (Acro Biologicals) was immobilized on DYNABEADS® His-Tag Isolation and Pulldown beads (Thermofisher). Following the first round of selection, a negative selection step was employed which was included in all the subsequent rounds. Starting at Round 4, iron-bound (diferric) hTf was included as a competitor at 12.5 μM to block aptamers from binding to this site on the protein. To ensure function on cells, two additional rounds of selection (Rounds 7 and 8) were conducted on Jurkat cells, a human T cell lymphoma line that expresses high levels of TfR using selection protocol that preferentially selects for compounds that have been endocytosed.

### B. Assessing the Progress of Selection

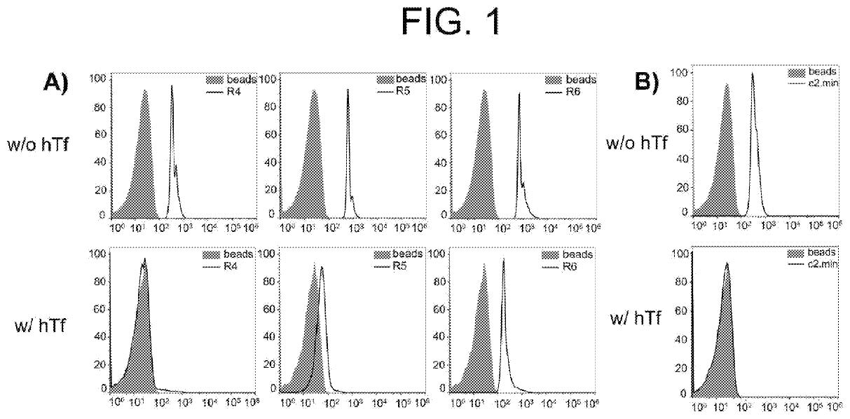

Flow cytometry was used to assess the progress of the selection using protein immobilized on beads as well as live cells. For bead-based assays, RNA from each round was labelled by hybridization with a fluorescently labelled reverse primer by heating at 85° C. for 3 minutes and allowing to cool at room temperature for 15 minutes. The libraries were subsequently incubated with bead-immobilized hTfR1 in SB1T buffer (40 mM HEPES, pH 7.5, 125 mM NaCl, 5 mM KCl, 1 mM MgCl2, 1 mM CaCl2, 0.05% Tween-20) containing 0.1% BSA and 1 μg/μl ssDNA. Following incubation for 60 minutes at 37° C., the beads were washed three times with SB1T, re-suspended in SB1T buffer and analyzed by flow cytometry. As shown in FIG. 1A, although bead staining by Round 4 was reduced to background by the addition of 12.5 μM hTf, Round 5 demonstrated increased activity in the presence of this competitor, which was further improved in Round 6. Importantly, when assayed under these same conditions, a previously identified anti-hTfR1 aptamer, c2.min, which is known to compete with hTf for binding (Wilner et al., 2012), shows no signal above untreated beads (FIG. 1B).

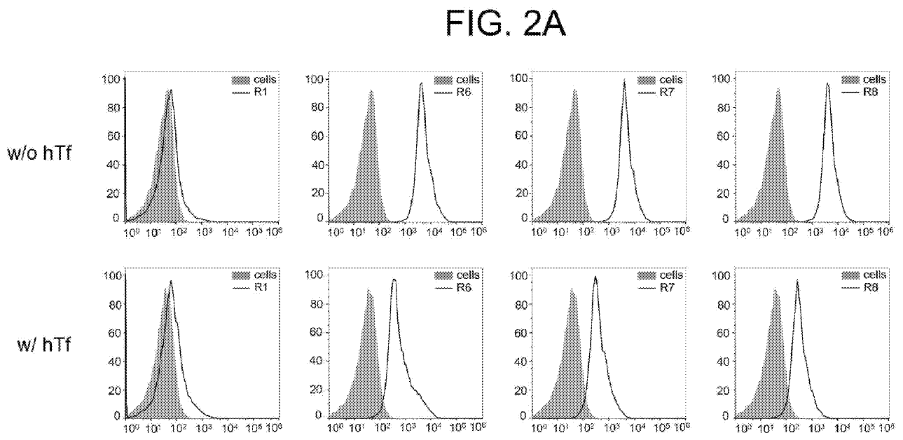

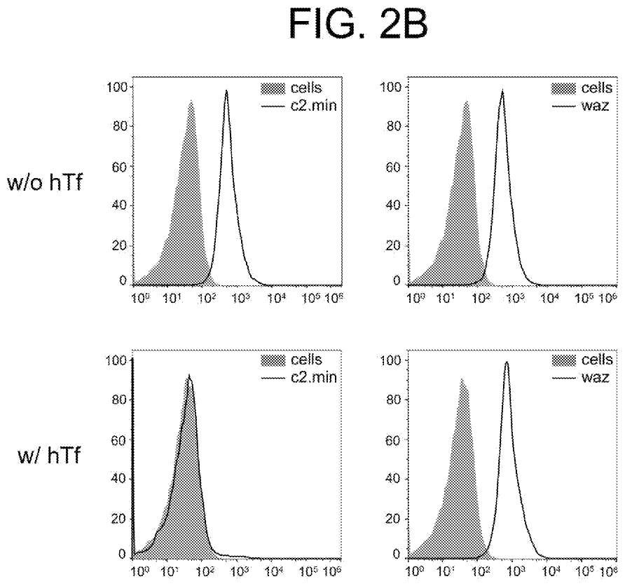

For cell-based assays, re-folded libraries were incubated with ~10⁵ Jurkat cells for 1 hr in complete RPMI media supplemented with 1 μg/μl ssDNA with or without 12.5 μM hTf. Following incubation for 60 minutes at 37° C., the cells were washed three times with FACS buffer [HBSS (Hanks Buffered Saline Solution)+1% BSA+0.1% NaAz] and analyzed by flow cytometry. Dead cells were excluded by the addition of Hoechst 33342. While Round 1 fails to effectively stain cells, Rounds 6, 7 and 8 all demonstrate robust cell staining even in the presence of 12.5 μM diferric hTf (FIG. 2A). For comparison, similar assays were performed using c2.min, which shows no activity in the presence of 12.5 μM diferric hTf, and Waz (Maier et al, 2016), an anti-hTfR1 aptamer that has previously been shown to bind hTfR1 independently of hTf (FIG. 2B).

### C. Selection, Purification and Characterization of Clones

The enriched aptamer populations recovered from Rounds 1 through 8 of the selection were sequenced using next-generation sequencing (NGS) to identify individual functional clones. Data from greater than ~20,000 individual sequences from each round were processed by trimming the flanking constant regions followed by alignment of the random region derived sequences. Aptamer sequences were ranked by frequency within each library and organized into families by clustering aptamers with similar sequence elements. All in silico analyses were performed using GENEIOUS® software (Biomatters Inc. Newark NJ, USA). From this analysis, 11 clones were chosen for further analysis and testing. A summary of the full-length clones identified for further testing from the selection is shown in Table 2. Aptamers composed of only the portion of the aptamer sequence derived from the random region, as listed in Table 3, were subsequently generated by chemical synthesis bearing either a 5′ or 3′ extension composed of an anti-MALAT1 ASO, linked to the aptamer portion by a DNA linker composed of 3 DNA residues, dT5mdCdA, such that aptamers with a 5′ extension have the sequence +G*+C*+A*T*T*5mdC*T*dA*dA*T*dA*dG*5mdC*+A*+G*+5mC-T5mdCdA-Aptamer (SEQ ID NO: 24) and those with a 3′ extension have the sequence Aptamer-dT5mdCdA-+G*+C*+A*T*T*5mdC*T*dA*dA*T*dA*dG*5mdC*+A*+G*+5mC (SEQ ID NO: 24). Aptamers composed of only the portion of the aptamer sequence derived from the random region bearing a 5′ amine (C6NH₂) for dye conjugation are listed in Table 4.

All aptamers (Table 3 and Table 4) were chemically synthesized on a Dr. Oligo 48X using the 2′-fluoro-G and 2′-O-methyl(2′OMe) A/C/U modified phosphoramidites, on a universal support. To facilitate downstream conjugations, some molecules (Table 4) were synthesized bearing a 5′ hexylamine linker (C6NH₂). All molecules were purified by trityl capture. For direct binding assays, synthesized aptamers were labeled with BP Fluor 647.

TABLE 2
Sequences of full-length TfR aptamers identified for further testing

| Clone ID | Sequence (5′ to 3′) |
|---|---|
| 36 | GGGAGAAUGCCGUAGCAGCGAGAAUAGUAUACGCAACCCGGUGGGAUAGG |
| | AUUAUCGGUUGCAUAAGCCAUUGUUAUACAUGGCUUGAGCCGGC |
| | (SEQ ID NO: 4) |
| 171 | GGGAGAAUGCCGUAGCAGCGAGAAUAGUCGACAGACCCGGAGGGAUAGGA |
| | UUAUCGGUCUUGAGAUGCAAACUUUAUACAUGGCUUGAGCCGGC |
| | (SEQ ID NO: 5) |
| 1 | GGGAGAAUGCCGUAGCAGCGAGAAUAGUCCCAUAGAUCCCGGAGGGAUAG |
| | GAAUUAUCGGAUCUAAACGUCUGUUAUACAUGGCUUGAGCCGGC (SEQ ID |
| | NO: 6) |
| 4 | GGGAGAAUGCCGUAGCAGCGAGAAUAGUCCAAUGGUCAACCGAACAGCGG |
| | AUAGCAUUGGGGUAGCGGUCGGUUUAUACAUGGCUUGAGCCGGC |
| | (SEQ ID NO: 7) |
| 27 | GGGAGAAUGCCGUAGCAGCGAGAAUAGUUGGGCGUCUCGCCAUUAUGACC |
| | CGGUGGGAUAGGAUUAUCGGUCUUUAUACAUGGCUUGAGCCGGC |
| | (SEQ ID NO: 8) |
| 7310 | GGGAGAAUGCCGUAGCAGCGAGAAUAGUCCGGACAGUCCACAGGUAGGAA |
| | AAGUUGUGGCUAAAUUGUCAACGUUAUACAUGGCUUGAGCCGGC |
| | (SEQ ID NO: 9) |
| 9350 | GGGAGAAUGCCGUAGCAGCGAGAAUAGUUCUGGGCGUACACAGAAACCUG |
| | CGGACUGUACGCUAAGAUGUCGGUUAUACAUGGCUUGAGCCGGC |
| | (SEQ ID NO: 10) |
| 12687 | GGGAGAAUGCCGUAGCAGCGAGAAUAGUGGUAGCAAACCGGUCUGAUAUG |
| | UUGUUAUGCGUUUGACCCUGUUGUUAUACAUGGCUUGAGCCGGC |
| | (SEQ ID NO: 11) |
| 3353 | GGGAGAAUGCCGUAGCAGCGAGAAUAGUACCCGCAGAGUCCGGAGUGUGU |
| | AGGUUAGCGGGCAUGGUAUGCAGUUAUACAUGGCUUGAGCCGGC |
| | (SEQ ID NO: 12) |
| R5g-457 | GGGAGAAUGCCGUAGCAGCGAGAAUAGCGCGGUGUGGAAAUGUAAAGAUC |
| | GUUGCCGCGAACGACGGUUGUUUUUAUACAUGGCUUGAGCCGGC |
| | (SEQ ID NO: 13) |
| R7g2-800 | GGGAGAAUGCCGUAGCAGCGAGAAUAGCGCGGUGUGGAAAUGUAAAGAUC |
| | GUUGCCGCGAACGACGGUUGUUUUUUAUACAUGGCUUGAGCCGGC |
| | (SEQ ID NO: 14) |

Where G is 2′F and A, C and U are 2′OMe modified RNA.

TABLE 3
Sequences of truncated hTfR1 aptamer-ASO conjugates

| Compound | | |
|---|---|---|
| Name | Aptamer Sequence (5′ to 3′) | 5′/3′ extension |
| AP-00030 | UAUACGCAACCCGGUGGGAUAGGAUUAUCGGUUGCAUAAGCCAUUGU | -T5mdCdA-00593- |
| | (SEQ ID NO: 15) | 3′ |
| AP-00031 | UCGACAGACCCGGAGGGAUAGGAUUAUCGGUCUUGAGAUGCAAACUU | -T5mdCdA-00593- |
| | (SEQ ID NO: 16) | 3 |
| AP-00032 | UCCCAUAGAUCCCGGAGGGAUAGGAAUUAUCGGAUCUAAACGUCUGU | -T5mdCdA-00593- |
| | (SEQ ID NO: 17) | 3′ |
| AP-00033 | UCCAAUGGUCAACCGAACAGCGGAUAGCAUUGGGGUAGCGGUCGGUU | -T5mdCdA-00593- |
| | (SEQ ID NO: 18) | 3′ |
| AP-00034 | UUGGGCGUCUCGCCAUUAUGACCCGGUGGGAUAGGAUUAUCGGUCUU | -T5mdCdA-00593- |
| | (SEQ ID NO: 19) | 3′ |
| AP-00035 | UCCGGACAGUCCACAGGUAGGAAAAGUUGUGGCUAAAUUGUCAACGU | -T5mdCdA-00593- |
| | (SEQ ID NO: 20) | 3′ |
| AP-00036 | UUCUGGGCGUACACAGAAACCUGCGGACUGUACGCUAAGAUGUCGGU | -T5mdCdA-00593- |
| | (SEQ ID NO: 21) | 3 |
| AP-00037 | UGGUAGCAAACCGGUCUGAUAUGUUGUUAUGCGUUUGACCCUGUUGU | -T5mdCdA-00593- |
| | (SEQ ID NO: 22) | 3′ |
| AP-00038 | UACCCGCAGAGUCCGGAGUGUGUAGGUUAGCGGGCAUGGUAUGCAGU | -T5mdCdA-00593- |
| | (SEQ ID NO: 23) | 3′ |
| AP-00039 | UAUACGCAACCCGGUGGGAUAGGAUUAUCGGUUGCAUAAGCCAUUGU | 5′-00593- |
| | (SEQ ID NO: 15) | T5mdCdA- |
| AP-00040 | UCGACAGACCCGGAGGGAUAGGAUUAUCGGUCUUGAGAUGCAAACUU | 5′-00593- |
| | (SEQ ID NO: 16) | T5mdCdA- |
| AP-00041 | UCCCAUAGAUCCCGGAGGGAUAGGAAUUAUCGGAUCUAAACGUCUGU | 5′-00593- |
| | (SEQ ID NO: 17) | T5mdCdA- |
| AP-00042 | UCCAAUGGUCAACCGAACAGCGGAUAGCAUUGGGGUAGCGGUCGGUU | 5′-00593- |
| | (SEQ ID NO: 18) | T5mdCdA- |
| AP-00043 | UUGGGCGUCUCGCCAUUAUGACCCGGUGGGAUAGGAUUAUCGGUCUU | 5′-00593- |
| | (SEQ ID NO: 19) | T5mdCdA- |
| AP-00044 | UCCGGACAGUCCACAGGUAGGAAAAGUUGUGGCUAAAUUGUCAACGU | 5′-00593- |
| | (SEQ ID NO: 20) | T5mdCdA- |
| AP-00045 | UUCUGGGCGUACACAGAAACCUGCGGACUGUACGCUAAGAUGUCGGU | 5′-00593- |
| | (SEQ ID NO: 21) | T5mdCdA- |
| AP-00046 | UGGUAGCAAACCGGUCUGAUAUGUUGUUAUGCGUUUGACCCUGUUGU | 5′-00593- |
| | (SEQ ID NO: 22) | T5mdCdA- |
| AP-00047 | UACCCGCAGAGUCCGGAGUGUGUAGGUUAGCGGGCAUGGUAUGCAGU | 5′-00593- |
| | (SEQ ID NO: 23) | T5mdCdA- |

Where G is 2′F and A, C and U are 2′OMe modified RNA; “00593” is an anti-MALAT1 ASO having the nucleoside sequence +G*+C*+A*T*T*5mdC*T*dA*dA*T*dA*dG*5mdC*+A*+G*+5mC (SEQ ID NO: 24) and “T5mdCdA” is a DNA linker, where dA, dG, T are DNA, 5mdC is 5-methyl deoxycytidine, 5mC is 5-methylcytidine, + denotes LNA, * is a phosphorothioate (PS) linkage

TABLE 4
Sequences of truncated hTfR1 aptamers

| Compound | | |
|---|---|---|
| name | 5′modification | Aptamer Sequence (5′ to 3′) |
| AP-00242 | C6NH2 | UAUACGCAACCCGGUGGGAUAGGAUUAUCGGUUGCAUAAGCCAUUGU |
| | | (SEQ ID NO: 15) |
| AP-00243 | C6NH2 | UCGACAGACCCGGAGGGAUAGGAUUAUCGGUCUUGAGAUGCAAACUU |
| | | (SEQ ID NO: 16) |
| AP-00244 | C6NH2 | UCCCAUAGAUCCCGGAGGGAUAGGAAUUAUCGGAUCUAAACGUCUGU |
| | | (SEQ ID NO: 17) |
| AP-00245 | C6NH2 | UUGGGCGUCUCGCCAUUAUGACCCGGUGGGAUAGGAUUAUCGGUCUU |
| | | (SEQ ID NO: 19) |

Where G is 2′F and A, C and U are 2′OMe modified RNA, and C6NH2 is a C6 alkylamine linker.

### D. Assaying Individual Synthesized Aptamers for Binding

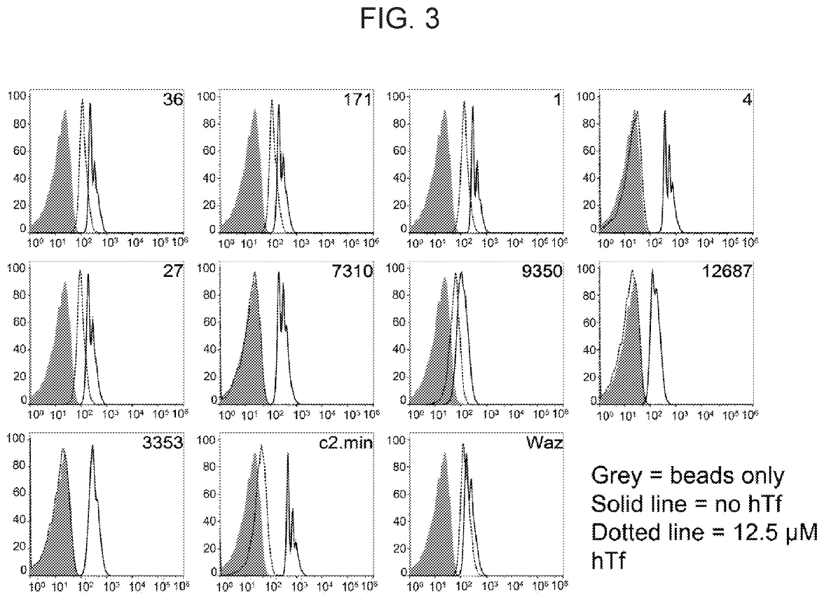

Full length aptamer sequences (Table 2) were ordered as DNA templates and amplified by PCR to generate dsDNA. The purified dsDNA was used as input for in vitro transcription reaction. RNA from each round was labelled by hybridization with a fluorescently labelled reverse primer by heating at 85° C. for 3 minutes and allowing to cool at room temperature for 15 minutes. Flow cytometry was then used to assess the activity of each aptamer clone on hTfR1 immobilized on beads in the presence or absence of 12.5 μM diferric hTf (FIG. 3). Under these assay conditions clones 36, 171, 1, 27 and 9350 all demonstrated signal even in the presence of 12.5 μM diferric hTf.

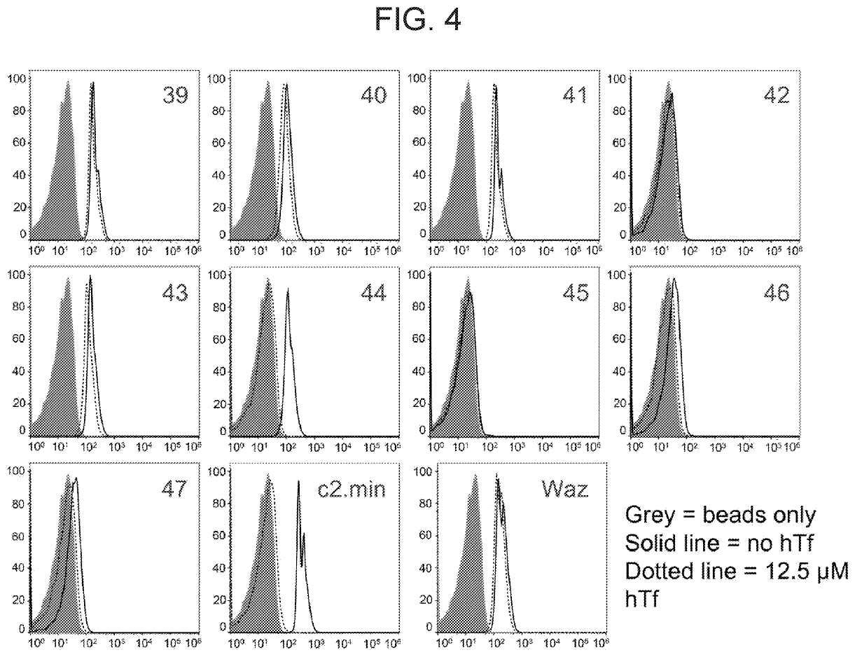

Modified RNA corresponding to only the random region of the aptamer clones identified in Table 2 were chemically synthesized bearing an anti-MALAT1 ASO at either the 3′ or 5′ end of each molecule with a T5mdCdA linker using standard solid phase chemical synthesis (Table 3). The chemically synthesized aptamers were subsequently labelled by hybridization with a DNA oligonucleotide complementary to anti-MALAT1 ASO bearing either a 5′ BP Fluor 647 (for hybridization to aptamers with a 3′ ASO) or a 3′ BP Fluor 647 (for hybridization to aptamers with a 5′ ASO). Aptamers and complementary oligonucleotides were thermally equilibrated by heating at 85° C. for 3 minutes at a molar ratio of 2:1 and allowed to cool for 15 minutes at room temperature. Flow cytometry was then used to assess the activity of each aptamer clone on hTfR1 immobilized on beads in the presence or absence of 12.5 μM diferric hTf. Data in FIG. 4 shows assays performed with aptamer bearing a 5′ ASO extension. Similar results were observed for aptamers generated with a 3′ extension (data not shown).

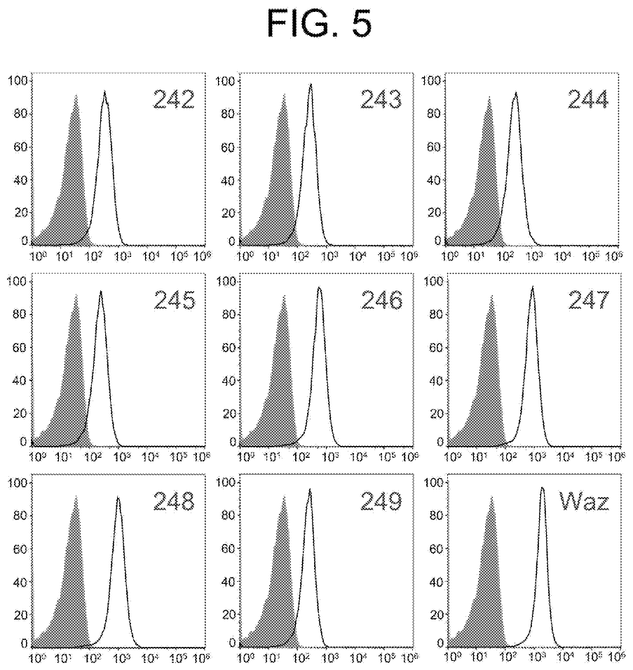

Modified RNA corresponding to only the random region of the aptamers 36, 171, 1 and 27 were chemically synthesized bearing 5′ amine using standard solid phase chemical synthesis (Table 4). The chemically synthesized aptamers were subsequently labelled at the 5′ end with BP Fluor 647 TFP (Broadpharm) via the terminal amine. Labelled aptamers we subsequently desalted to remove free dye were then assayed for target binding on Jurkat cells by flow cytometry. Briefly, aptamers were heated at 85° C. for 3 minutes, allowed to cool for 15 minutes at room temperature, and then incubated with 10⁵ Jurkat cells in full RPMI media for 60 minutes at 37° C. Following incubation, the cells were washed three times with FACS buffer (HBSS+1% BSA+0.1% NaAz) and analyzed by flow cytometry. Dead cells were excluded by the addition of Hoechst 33342. As shown in FIG. 5, the directly labelled aptamers, including the truncates, maintained hTfR1 binding activity.

### Example 2: N45—2nd Approach

A. Selection for Nuclease Stabilized (fGmH) Anti-hTfR Aptamers

Using the N45 library (Table 1), an alternative approach was used to identify anti-hTfR1 aptamers. Briefly, we initiated a multiplex selection targeting hTfR1 and 4 other undisclosed protein targets, all immobilized on DYNABEADS® His-Tag Isolation and Pulldown beads (Thermofisher). Following an initial 3 rounds of selection, the library was challenged for an additional 6 rounds of selection, binding to and internalizing into Jurkat cells in the presence of 12.5 μM diferric hTf.

Following the first round of selection, a negative selection step was employed which was included in all the subsequent rounds of selection using beads. For the negative selection, the pool was prepared as described above and then was incubated with beads only for 30 minutes at 37° C. in SB1T buffer. The beads were then spun down and the supernatant, containing molecules that did not bind to the unlabeled beads, was utilized for the positive selection step.

Following the first 3 rounds of selection on recombinant protein, 6 additional rounds of selection were conducted on Jurkat cells, a human T cell lymphoma line that expresses high levels of hTfR1 using selection protocol that preferentially selects for compounds that have been endocytosed (Maier et al, 2016).

### B. Assessing the Progress of Selection

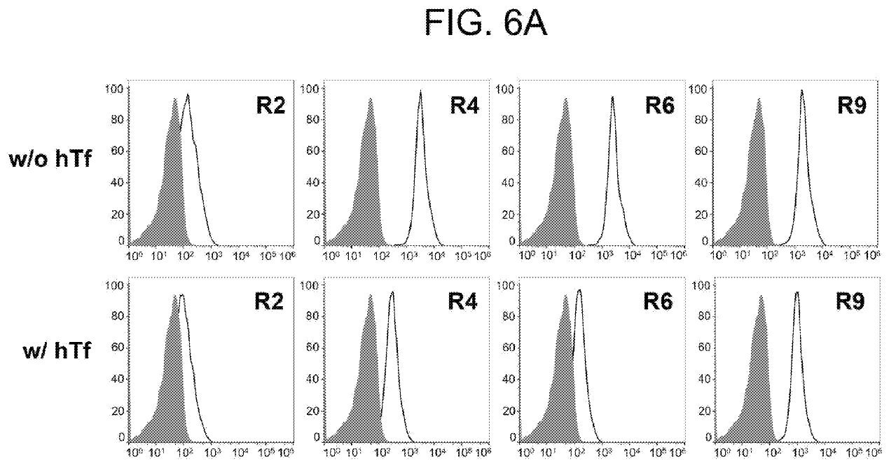

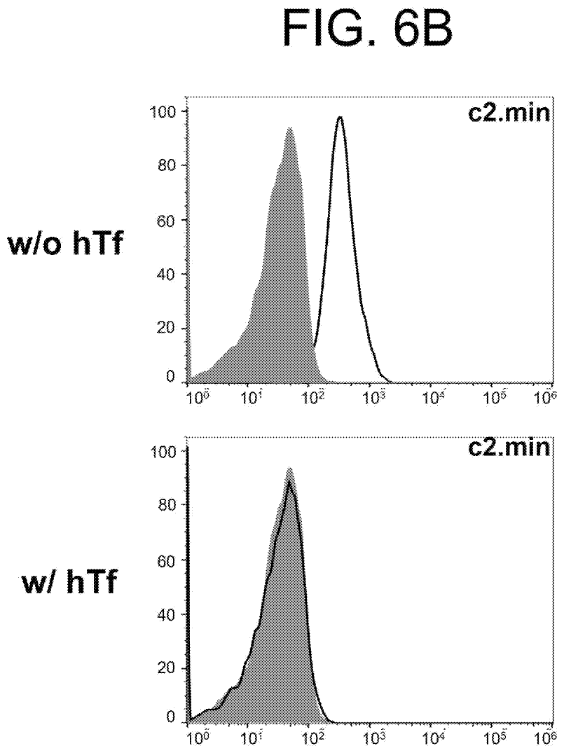

Flow cytometry was used to assess the progress of the selection using live cells. For bead-based assays, RNA from each round was labelled by hybridization with a fluorescently labelled reverse primer by heating at 85° C. for 3 minutes and allowing to cool at room temperature for 15 minutes. Libraries were then incubated with 10⁵ Jurkat cells in full RPMI media for 60 minutes at 37° C. with or without 12.5 μM hTf. Following incubation, the cells were washed three times with FACS buffer (HBSS+1% BSA+0.1% NaAz) and analyzed by flow cytometry. Dead cells were excluded by the addition of Hoechst 33342. As shown in FIG. 6, successive rounds of selection led to an increase in cell staining even in the presence of 12.5 μM diferric hTf.

### C. Selection, Purification and Characterization of Clones

The enriched aptamer populations recovered from Rounds 1 through 9 of the selection were sequenced using next-generation sequencing (NGS) to identify individual functional clones. Data from greater than ~20,000 individual sequences from each round were processed by trimming the flanking constant regions followed by alignment of the random region derived sequences. Aptamer sequences were ranked by frequency within each library and organized into families by clustering aptamers with similar sequence elements. All in silico analyses were performed using GENEIOUS® software (Biomatters Inc. Newark NJ, USA). From this analysis we identified a single dominant clone for additional analysis (R9JDS-1; Table 5). While this sequence bears 6 Us at the end of the random region, numerous other versions of this compound appeared in the population that were identical to R9JDS-1 but possessed between 3 and 16 terminal Us. Interestingly, similar variants were identified during the previous N45 selection containing 4 and 5 Us (Example 1; R5g-457 and R7g2-800; Table 2). To confirm the function of this compound, we chemically synthesized variants of R9JDS-1 comprised of only the random region of the library and made compounds bearing 4 or 6 terminal Us. All compounds were synthesized bearing either a 5′ or 3′ extension composed of an anti-MALAT1 ASO, linked to the aptamer portion by a DNA linker composed of 3 DNA residues, dT5mdCdA, such that aptamers with a 5′ extension have the sequence (Table 6).

TABLE 5
Sequences of full-length TfR aptamers identified for further testing

| Clone ID | Sequence (5′ to 3′) |
|---|---|
| R9JDS-1 | GGGAGAAUGCCGUAGCAGCGAGAAUAGCGCGGUGUGGAAAUGUAAAGAUCGUUGCCGCGAACGACGGUUGUUUUUUUAUA |
| | CAUGGCUUGAGCCGGC (SEQ ID NO: 25) |

Where G is 2′F and A, C and U are 2′OMe modified RNA.

TABLE 6
Sequences of core TfR aptamers with ASO

| Compound Name | Aptamer Sequence (5′ to 3′) | extension |
|---|---|---|
| AP-00257 | CGCGGUGUGGAAAUGUAAAGAUCGUUGCCGCGAACGACGGUUGUUUU | 5′00593-T5mdCdA- |
| | (SEQ ID NO: 26) | |
| AP-00258 | CGCGGUGUGGAAAUGUAAAGAUCGUUGCCGCGAACGACGGUUGUUUU | -T5mdCdA-00593-3′ |
| | (SEQ ID NO: 26) | |
| AP-00259 | CGCGGUGUGGAAAUGUAAAGAUCGUUGCCGCGAACGACGGUUGUUUUUU | 5′00593-T5mdCdA- |
| | (SEQ ID NO: 27) | |
| AP-00260 | CGCGGUGUGGAAAUGUAAAGAUCGUUGCCGCGAACGACGGUUGUUUUUU | -T5mdCdA-00593-3′ |
| | (SEQ ID NO: 27) | |

Where G is 2′F and A, C and U are 2′OMe modified RNA; “00593” is an anti-MALAT1 ASO having the nucleoside sequence +G*+C*+A*T*T*5mdC*T*dA*dA*T*dA*dG*5mdC*+A*+G*+5mC (SEQ ID NO: 24) and “T5mdCdA” is a DNA linker, where dA, dG, T are DNA, 5mdC is 5-methyl deoxycytidine, + denotes LNA, * is a phosphorothioate (PS) linkage

### D. Assaying Individual Synthesized Aptamers for Binding

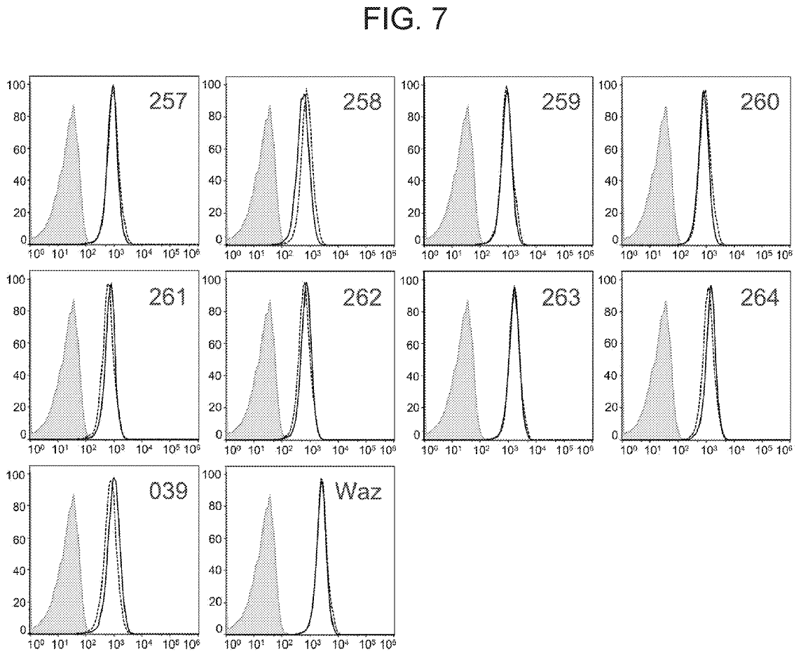

Modified RNA corresponding to only the random region of the aptamer clone identified in Table 5 with 4 or 6 terminal Us was chemically synthesized bearing an anti-MALAT1 ASO at either the 3′ or 5′ end of each molecule with a T5mdCdA linker using standard solid phase chemical synthesis (Table 6). The chemically synthesized aptamers were subsequently labelled by hybridization with a DNA oligonucleotide complementary to anti-MALAT1 ASO bearing either a 5′ BP Fluor 647 (for hybridization to aptamers with a 3′ ASO) or a 3′ BP Fluor 647 (for hybridization to aptamers with a 5′ ASO). Aptamers and complementary oligonucleotides were thermally equilibrated by heating at 85° C. for 3 minutes at a molar ratio of 2:1 and allowed to cool for 15 minutes at room temperature. Flow cytometry was then used to assess the activity of each aptamer clone on hTfR1 Jurkat cells in the presence or absence of 12.5 μM diferric hTf (FIG. 7). For comparison, assays were also run using Waz and AP-0039 (Table 3). All compounds show activity in the presence of 12.5 μM hTf.

### Example 3: N45—3rd Approach

A. Selection for Nuclease Stabilized (fGmH) Anti-hTfR Aptamers

Using an N35 library (Table 7), an alternative approach was used to identify anti-hTfR aptamers. Briefly, we initiated a selection targeting hTfR1 bearing a N-terminal His-tag on DYNABEADS® His-Tag Isolation and Pulldown beads (Thermofisher). Following an initial 3 rounds of selection, the library was challenged for an additional 8 rounds of selection, binding to and internalizing into Jurkat cells in the presence of 12.5 μM diferric hTf.

TABLE 7
Library sequence and sequence of oligos used to amplify the N35 library

| | Sequence (5′ to 3′) |
|---|---|
| Library sequence | GGGAGAGTCGGTAGCAGTC-T-N35-T-CTATGTGGAAATGGCGCTGT |
| (Total library length: | (SEQ ID NO: 28) |
| 76 bases) | |
| N35.F | GGGAGAGTCGGTAGCAGTC (SEQ ID NO: 29) |
| N35.R | ACAGCGCCATTTCCACATAG (SEQ ID NO: 30) |

Where G, A, T and C are deoxyribonucleotides; “N35” is a 35-nucleotide random region; the N35-F primer also includes a 5′ phage polymerase promoter.

### B. Assessing the Progress of Selection

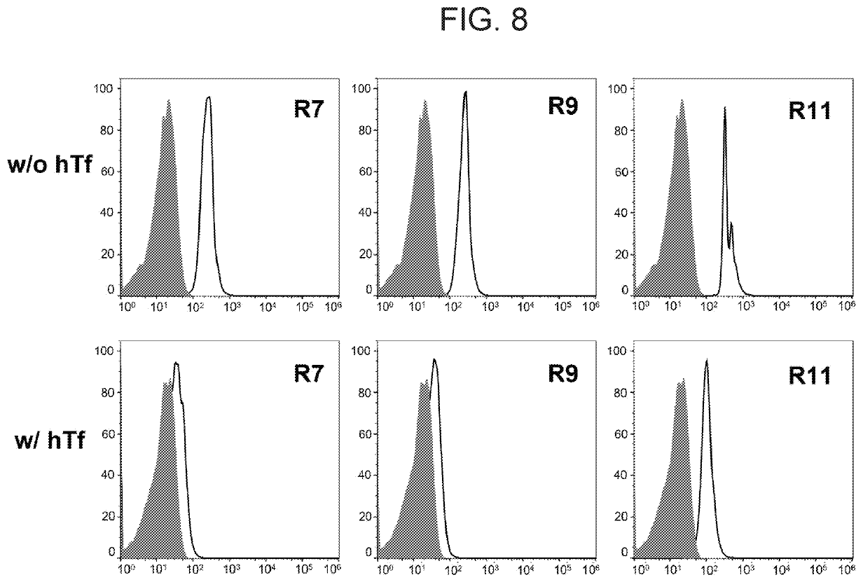

Flow cytometry was used to assess the progress of the selection using protein immobilized on beads as well as live cells. Briefly, RNA from each round was labelled with a fluorescently labelled reverse primer by heating at 85° C. for 3 minutes and allowed to cool at room temperature for 15 minutes. The libraries were subsequently incubated with hTfR1 immobilized on His-Tag Isolation and Pulldown beads (Thermofisher) in the presence or absence of 12.5 uM diferric hTf. Following incubation, beads were washed, and binding was assessed by flow cytometry (FIG. 8).

### C. Selection, Purification and Characterization of Clones

The enriched aptamer populations recovered from Rounds 1 through 11 of the selection were sequenced using next-generation sequencing (NGS) to identify individual functional clones. Data from greater than ~20,000 individual sequences from each round were processed by trimming the flanking constant regions followed by alignment of the random region derived sequences. Aptamer sequences were ranked by frequency within each library and organized into families by clustering aptamers with similar sequence elements. All in silico analyses were performed using GENEIOUS® software (Biomatters Inc. Newark NJ, USA). From this analysis, clones were chosen for further analysis and testing. A summary of the full-length clones is shown in Table 8.

TABLE 8
Sequences of full-length TfR aptamers identified for further testing

| Clone ID | Sequence (5′ to 3′) |
|---|---|
| MW_R11EDS#1_full | GGGAGAGUCGGUAGCAGUCUGUAGAAAGGUAACGAUCGUUGGACUCACUGUAAUCUCUAUGUGGAAAUGGC |
| | GCUGU (SEQ ID NO: 31) |
| MW_R11EDS#4_full | GGGAGAGUCGGUAGCAGUCUUGGUGGUCCCAGGGUUGUUGGUUGCUCGUCCCGUAUCUAUGUGGAAAUGGC |
| | GCUGU (SEQ ID NO: 32) |
| MW_R11EDS#10_full | GGGAGAGUCGGUAGCAGUCUGCAGGUGCCUGCGGCUGUUGCUUGCUUUGUCGUUUUCUAUGUGGAAAUGGC |
| | GCUGU (SEQ ID NO: 33) |
| MW_R11EDS#13_full | GGGAGAGUCGGUAGCAGUCUUGGCCGCAGGGUUGUUGUUUGCUCUUCCGUAGUUCUCUAUGUGGAAAUGGC |
| | GCUGU (SEQ ID NO: 34) |
| MW_R11EDS#16_full | GGGAGAGUCGGUAGCAGUCUUUGGCGAAAUCAGGUUCUGUUGUUUGCUUUACCCGUCUAUGUGGAAAUGGC |
| | GCUGU (SEQ ID NO: 35) |
| MW_R11EDS#24_full | GGGAGAGUCGGUAGCAGUCUGCUGCAGGUAACUGUUGUCCUGUUGUUUGUUCUCCUCUAUGUGGAAAUGGC |
| | GCUGU (SEQ ID NO: 36) |

where G is 2′F and A, C and U are 2′OMe modified RNA

TABLE 9
Sequence of core hTfR1 aptamer

| Compound | | |
|---|---|---|
| Name | 5′ modification | Sequence (5′ to 3′) |
| AP-00353 | C6NH2 | GGGAGAGUCGGUAGCAGUCUGUAGAAAGGUAACGAUCGUUGGACUCACUGUAAUCU |
| | | (SEQ ID NO: 37) |

Where G is 2′F and A, C and U are 2′OMe modified RNA and C6NH2 is a C6 hexylamine linker.

### D. Assaying Individual Synthesized Aptamers for Binding

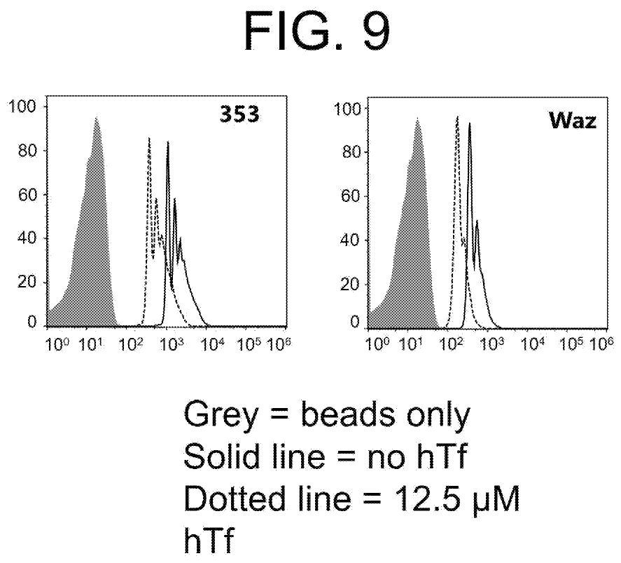

Modified RNA corresponding to only the random region of MW_R11EDS#1_full was chemically synthesized bearing 5′ amine using standard solid phase chemical synthesis (Table 9). The chemically synthesized aptamer was subsequently labelled at the 5′ end with BP Fluor 647 TFP (Broadpharm) via the terminal amine. The labelled aptamer was desalted to remove free dye and then assayed for target binding on hTfR1 immobilized on beads. Briefly, the aptamer was heated at 85° C. for 3 minutes, allowed to cool for 15 minutes at room temperature and then incubated with beads. Following incubation, the beads were washed and analyzed by flow cytometry. As shown in FIG. 9, the directly labelled aptamer maintained hTfR1 binding activity.

### Example 4. Sequence Family Analysis and Minimization of Anti-hTfR1 Aptamers

| | | Corresp- | |
|---|---|---|---|
| | | onding | Corresp- |
| | | aptamer- | onding |
| Aptamer | | 5′ ASO | 5′ amine |
| SEQ | | conjugate | aptamer |
| ID NO. | Aptamer Sequence (5′ to 3′) | (Table 3) | (Table 4) |
| 15 | ------------UAUACGCAACCCGGUGGGAUAGGA- | AP-0039 | AP-00242 |
| | UUAUCGGUUGCAUAAGCCAUUGU- | | |
| 16 | -------------UCGACAGACCCGGAGGGAUAGGA- | AP-0040 | AP-00243 |
| | UUAUCGGUCUUGAGAUGCAAACUU | | |
| 17 | -----------UCCCAUAGAUCCCGGAGGGAUAGGAAUUAUCGGAUCUAAACGUCUGU-- | AP-0041 | AP-00244 |
| | - | | |
| 19 | UUGGGCGUCUCGCCAUUAUGACCCGGUGGGAUAGGA-UUAUCGGUCUU------------ | AP-0043 | AP-00245 |
| | - | | |

| | | |
|---|---|---|
| 38 | XCCCGGWGGGAUAGGAOUUAUCGGZ | N/A |
| | | (shared core motif) |

The shared core is in bold with the adjacent closing stem underlined. G is 2′F and A, C and U are 2′OMe modified RNA; W is A or U, O is an A or a deletion, and X and Z are independently a sequence of 2-5 linked nucleosides that interact to form a stem of 2 to 5 nucleotides

### A. AP-00039/AP-00248 Family

Sequence analysis of aptamers identified in Example 1 reveals a family of related compounds which share a common core motif, X(2-5)CCCGGWGGGAUAGGAOUUAUCGGZ(2-5) (SEQ ID NO: 38), where W is A or U, O is an A or a deletion, and X(2-5) and Z(2-5) are each a sequence of 2-5 linked nucleosides that interact to form a stem of 2 to 5 nucleotides (Table 10).

### Table 10. Members of Aptamer AP-00039 Family

It is important to note that although these sequences share a common 22-23 nt core, the flanking sequences for each of these compounds are unique, indicating that each of these aptamers arose independently during the selection. As such, this motif can be considered a universal hTfR1 binding motif.

Consistent with this, aptamer truncates designed using the flanking stem sequence from SEQ ID NO: 15 (AP-00039) (Table 11: underlined) and the cores from SEQ ID NOs: 15 (AP-00039), 16 (AP-00040) and 17 (AP-00041) (Table 11: bold) demonstrated robust function in flow cytometry-based assays (FIG. 5). As further confirmation, similar activity was observed when assays were performed using AP-00249, a construct derived from the core region of SEQ ID NO: 15 (AP-00039) flanked by a stable 3 base-pair closing stem GCG/CGC not found in SEQ ID NO: 15 (AP-00039), 16 (AP-00040) or 17 (AP-00041) (Table 11; FIG. 5).

TABLE 11
Aptamer truncates based on the AP-00039 Family

| | | | Aptamer | | Flanking |
|---|---|---|---|---|---|
| Compound | | Aptamer truncate Sequence | (truncate) | Core | Stem |
| Number | 5′mod | (5′ to 3′) | SEQ ID NO | Parent | Parent |
| AP-00039 | ASO | UAUACGCAACCCGGUGGGAUAGGA- | 15 | N/A | N/A |
| | | UUAUCGGUUGCAUAAGCCAUUGU | | | |
| AP-00246 | C6NH2 | GCAACCCGGUGGGAUAGGA-UUAUCGGUUGC | 39 | AP- | AP- |
| | | | | 00039 | 00039 |
| AP-00247 | C6NH2 | GCAACCCGGAGGGAUAGGA-UUAUCGGUUGC | 40 | AP- | AP- |
| | | | | 00040 | 00039 |
| AP-00248 | C6NH2 | GCAACCCGGAGGGAUAGGAAUUAUCGGUUGC | 41 | AP- | AP- |
| | | | | 00041 | 00039 |
| AP-00249 | C6NH2 | GCGCCCGGUGGGAUAGGA-UUAUCGGCGC | 42 | AP- | de novo |
| | | | | 00039 | |

Where G is 2′F and A, C and U are 2′OMe modified RNA.

For these experiments, modified RNAs were chemically synthesized bearing 5′ amine using standard solid phase chemical synthesis (Table 11). The chemically synthesized aptamers were subsequently labelled at the 5′ end with BP Fluor 647 TFP (Broadpharm) via the terminal amine. Labelled aptamers were desalted to remove free dye and then assayed for target binding on Jurkat cells by flow cytometry. Briefly, aptamers were heated at 85° C. for 3 minutes, allowed to cool for 15 minutes at room temperature and then incubated with 105 Jurkat cells in full RPMI media for 60 minutes at 37° C. Following incubation, the cells were washed three times with FACS buffer (HBSS+1% BSA+0.1% NaAz) and analyzed by flow cytometry. Dead cells were excluded by the addition of Hoechst 33342. As shown in FIG. 5, the directly labelled aptamers, including the truncates, maintained hTfR1 binding activity as well or better than the corresponding parent compound, AP-0039 (Table 5).

### B. AP-00264/AP-00353 Family

Sequence analysis of the lead aptamers identified from the N45 library in Example 2 (AP-00257 through AP-00260) revealed a striking similarity with AP-00353 identified from the selection performed using an N35 selection in Example 3, and revealed a common core composed of the sequence X(4-5)UGURGAAAKGUAAMGAUCGUUGZ(4-5) (SEQ ID NO: 43), where R is A or G, K is G or U, M is A or C, and X(4-5) and Z(4-5) are each independently a sequence of 4-5 linked nucleosides that interact to form a stem (Table 12). The underlined nucleotides interact to form a stem of 5 or 6 nucleotides. Here again, it is important note that as with the common core observed within the 248-family, which appeared embedded within different sequences, the shared 22 nt core in the 264/353 family was found embedded in different sequences identified from separate selections using two completely different libraries. As such, this motif can also be considered a universal hTfR1 binding motif.

TABLE 12
Members of Aptamer AP-00264/AP-00353 Family

| | | Corresponding | Corres- |
|---|---|---|---|
| | | aptamer- | ponding |
| | | 5′ ASO | 5′ amine |
| Aptamer | | conjugate | aptamer |
| SEQ ID NO | Sequence (5′ to 3′) | (Table 6) | (Table 9) |
| SEQ ID NO: 26 | -------------- | AP-00257 | N/A |
| | CGCGGUGUGGAAAUGUAAAGAUCGUUGCCGCGAACGACGGUUGUUUU | | |
| SEQ ID NO: 27 | -------------- | AP-00259 | N/A |
| | CGCGGUGUGGAAAUGUAAAGAUCGUUGCCGCGAACGACGGUUGUUUU | | |
| | UU | | |
| SEQ ID NO: 37 | GGGAGAGUCGGUAGCAGUCUGUAGAAAGGUAACGAUCGUUGGACUCA | N/A | AP-00353 |
| | CUGUAAUCU-------- | | |

| | | |
|---|---|---|
| SEQ ID NO: 43 | XUGURGAAAKGUAAMGAUCGUUGZ | N/A |
| | | (shared core motif) |

The shared core is in bold with the adjacent closing stem underlined, where R is A or G, K is G or U, M is A or C, X and Z are independently a sequence of 4-5 linked nucleosides that interact to form a stem

Consistent with this, aptamer truncates containing this core structure (Table 13; AP-00259 through AP-00264) maintain activity levels similar to the parent compounds (AP-00257 through AP-00260; FIG. 7).

TABLE 13
Core sequences of AP-00264/AP-00353 family hTfR1 aptamers with ASO

| Compound | | Aptamer | 5′/3′ |
|---|---|---|---|
| Name | Aptamer Sequence (5′ to 3′) | SEQ ID NO. | extension |
| AP-00259 | CGCGGUGUGGAAAUGUAAAGAUCGUUGCCGCGAACGACG | SEQ ID NO: 27 | 5′00593- |
| | GUUGUUUUUU | | T5mdCdA- |
| AP-00261 | CGCGGUGUGGAAAUGUAAAGAUCGUUGCCGCGAACGACG | SEQ ID NO: 44 | 5′00593- |
| | | | T5mdCdA- |
| AP-00262 | CGCGGUGUGGAAAUGUAAAGAUCGUUGCCGCGAACGACG | SEQ ID NO: 44 | -UT5mdCdA- |
| | | | 00593-3′ |
| AP-00263 | CGCGGUGUGGAAAUGUAAAGAUCGUUGCCGCG | SEQ ID NO: 45 | 5′00593- |
| | | | T5mdCdA- |
| AP-00264 | CGCGGUGUGGAAAUGUAAAGAUCGUUGCCGCG | SEQ ID NO: 45 | -UT5mdCdA- |
| | | | 00593-3′ |

Where G is 2′F and A, C and U are 2′OMe modified RNA. The shared core is in bold with the adjacent closing stem underlined. “00593” and “T5mdCdA” are as defined in Table 6; “UT5mdCdA” corresponds to T5mdCdA with an additional U-2′OMe nucleoside at its 3′ terminus

For these experiments, truncates listed in Table 13 were chemically synthesized bearing an anti-MALAT1 ASO at either the 3′ or 5′ end of each molecule with a (U)T5mdCdA linker using standard solid phase chemical synthesis and subsequently labelled by hybridization with a DNA oligonucleotide complementary to anti-MALAT1 ASO bearing either a 5′ BP Fluor 647 (for hybridization to aptamers with a 3′ ASO) or a 3′ BP Fluor 647 (for hybridization to aptamers with a 5′ ASO). Aptamers and complementary oligonucleotides were thermally equilibrated by heating at 85° C. for 3 minutes at a molar ratio of 2:1, allowed to cool for 15 minutes at room temperature and then incubated with 10⁵ Jurkat cells in full RPMI media for 60 minutes at 37° C. Following incubation, the cells were washed three times with FACS buffer (HBSS+1% BSA+0.1% NaAz) and analyzed by flow cytometry (FIG. 7).

### Example 5. Degenerate Aptamer Selection—AP-00353

### A. Degenerate Selection for AP-00353 Variants.

In order to better understand the sequence and structure-function relationships within AP-00264 and AP-00353 family members we performed a degenerate selection based on the sequence of AP-00353 using a synthetic library doped at 70% (Table 14).

TABLE 14
Library sequence and sequence of oligos used to amplify the library

| | Sequence (5′ to 3′) |
|---|---|
| Library sequence | GGGAGCGTAGCTAGTATCCGGGAGAGTCGGTAGCAGTCTGT |
| (Total library | AGAAAGGTAACGATCGTTGGACTCACTGTAATCTCTATGTG |
| length: 95 bases) | GAAATGGCGCTGT (SEQ ID NO: 46) |
| d353.F | GGGAGCGTAGCTAGTATCC (SEQ ID NO: 47) |
| N35.R | ACAGCGCCATTTCCACATAG (SEQ ID NO: 30) |

Where G, A, T and C are deoxyribonucleotides and the d353.F primer also includes a 5′ phage polymerase promoter. Underlined bolded positions indicate the degenerate library where the identity of each position is 70% the base indicated and 10% the remaining 3 nucleotides.

Briefly, using a fully backbone modified fGmH RNA library, we initiated a selection targeting hTfR1 immobilized on DYNABEADS® His-Tag Isolation and Pulldown beads (Thermofisher). The amount of target protein, input RNA, blocking agents and washing conditions varied between rounds. Following an initial 3 rounds of selection the library was challenged for an additional 3 rounds of selection, binding to (Rounds 4 and 5) and internalizing into (Round 6) Jurkat cells in the presence of 12.5 μM diferric hTf.

### B. Identification and Characterization of Selected Sequences

Following Round 6, the selected library displayed activity consistent with or better than the parent compound (data not shown) and the enriched aptamer populations recovered from Rounds 1 through 6 of the selection were sequenced using next-generation sequencing (NGS) to identify individual functional clones. Data from greater than ~20,000 individual sequences from each round were processed by trimming the flanking constant regions followed by alignment of the random region derived sequences. Aptamer sequences were ranked by frequency within each library and organized into families by clustering aptamers with similar sequence elements. All in silico analyses were performed using GENEIOUS® software (Biomatters Inc. Newark NJ, USA). From this analysis, clones were chosen for further analysis and testing. Aptamers composed of only the portion of the aptamer sequence derived from the random region or truncations thereof (listed in Table 15), were subsequently generated by chemical synthesis bearing a 5′ terminal amine to facilitate dye conjugation and assayed for function on Jurkat cells or protein immobilized on beads as described above. Assays were performed over a range of aptamer concentrations (0.02, 0.05, 0.15, 0.46, 1.37, 4.1, 12.43, 111, 333, 1000, 3000 nM) and apparent binding constants (Kₐₚₚ) determined by a fit with the equation y=(Kₐₚₚ*[x])/(Bₘₐₓ+[x]). Relative binding affinity was calculated by dividing the apparent binding constant (Kₐₚₚ) for each compound by that determined for AP-00403, the 5′ C6 terminal amine (C6NH2) variant of AP-00264.

TABLE 15
Relative affinities of binding to protein beads and Jurkat cells

| | | Aptamer | Relative | Relative |
|---|---|---|---|---|
| Compound | | SEQ ID | Affinity | Affinity |
| Name | Aptamer Sequence (5′ to 3′) | NO. | Protein Beads | Cells |
| AP-00403 | CGCGGUGUGGAAAUGUAAAGAUCGUUGCCGCG | SEQ ID NO: 45 | 1.0 | 1.0 |
| AP-00353 | GGGAGAGUCGGUAGCAGUCUGUAGAAAGGUAACG | SEQ ID NO: 37 | nd | 2.1 |
| | AUCGUUGGACUCACUGUAAUCU | | | |
| AP-00382 | CGGAGACUCGGUAGACUUACGUAGAAAGGUAACG | SEQ ID NO: 48 | 0.02 | 0.16 |
| | AUCAUUGUGUCGUAUCGAUUCG | | | |
| AP-00388 | GGCAGAGUCGGUGGGCUUACGUAGAAAGGUAACG | SEQ ID NO: 49 | 0.01 | 0.14 |
| | AUCAUUGUGCUCACUGUCAUGU | | | |
| AP-00389 | UGAAAAAUUAGUCGGCUUACGUAGAAAGGUAACG | SEQ ID NO: 50 | 0.03 | 0.16 |
| | AUCAUUGUGCUGACUGAAGUCU | | | |
| AP-00390 | AGUUCCUUACGUAGCUCUUGACGUA- | SEQ ID NO: 51 | 0.0004 | 20 |
| | AAAGAUCACGAUUGUGGGAGUGACAGGAACCG | | | |
| AP-00392 | CGUAGACGAUUUACCGGUCCGUAGAAAUGUAAAG | SEQ ID NO: 52 | 0.25 | nd |
| | AUCGUUGGACUCGGUGUCUACU | | | |
| AP-00385 | cgGACUUACGUAGAAAGGUAACGAUCAUUGUGUC | SEQ ID NO: 53 | 0.02 | 0.13 |
| | cg | | | |
| AP-00414 | cgcGACUUACGUAGAAAGGUAACGAUCAUUGUGU | SEQ ID NO: 54 | 0.01 | 0.15 |
| | Cgcg | | | |
| AP-00074 | GGGUUCUACGAUAAACGGUUAAUGAUCAGCUUAU | SEQ ID NO: 55 | 0.02 | 0.18 |
| | GGCUGGCAGUUCCC | | | |

Where all compounds bear a 5′ C6 terminal amine (C6NH2) and G is 2′F and A, C and U are 2′OMe modified RNA for all compounds except AP-00074, where G and A are 2′ OH and C and U are 2′F. The shared core is in bold with the adjacent closing stem underlined. The inserted conserved UU is highlighted in italics and the U to Cat position 1 of core in bold italics. Relative affinities against Jurkat cells or protein on beads were determined by flow cytometry using fluorescently labelled aptamers generated by direct dye conjugation (BP Fluor 647). All values are reported relative to AP-00403, the 5′ C6 terminal amine (C6NH2) variant of AP-00264.

When compared to AP-00403 (the 5′ C6 terminal amine (C6NH2) variant of AP-00264), aptamers AP-00382, AP-00388, and AP-00389 demonstrated a significant affinity enhancement (Table 15). Sequence analysis revealed that these variants possess the addition of a novel conserved feature, a UU bulge. That is, compared to AP-00264 and AP-00353, in which the conserved aptamer core was flanked immediately by a stem X(4-5)UGURGAAAKGUAAMGAUCGUUGZ(4-5) (SEQ ID NO: 43), where R is A or G, K is G or U, M is A or C, and X(4-5) and Z(4-5) are each independently a sequence of 4-5 linked nucleosides that interact to form a stem, these improved variants have a UU bulge inserted within the stem (Table 15, italic UU). These compounds also have a change in the first position of the core from a U to a C and the third to last position in the core from a G to an A. The extended core sequence for the 264/353/385 family can thus be written as

(SEQ ID NO: 56)
X₁(3-9)UUX₂YGURGAAAKGUAAMGAUCRUUGZ₂Z₁(4-10),

where Y is Cor U, R is A or G, K is G or U, M is A or C, X is any nucleoside, X(3-9) and Z₁(4-10) are each independently a sequence of 3-9 or 4-10 linked nucleosides respectively, and X₂ and X₁(3-9) are together a stem-forming region that interacts with Z₁ (4-10) to form a stem, and the terminal stem underlined (Table 15). As further confirmation, AP-00385 and AP-00414 which are comprised of only the core and flanking stem of AP-00382,

(SEQ ID NO: 57)
GACUUACGUAGAAAGGUAACGAUCAUUGUGUC,

maintain complete activity with the addition of two base pair closing stem (cg/cg) or three base pair closing stem (cgc/gcg). A consensus for these sequences could be written as

(SEQ ID NO: 58)
S(2-3)GACUUACGUAGAAAGGUAACGAUCAUUGUGUCS′(2-3),

where S(2-3) and S′(2-3) are each a sequence of 2-3 linked nucleosides, each of which are each independently G or C nucleosides, and S(2-3) pairs with S′(2-3) to form a stem, and the terminal stem is underlined.

### Example 6. In Vitro siRNA Delivery Using Anti-hTfR1 Aptamers

Anti-hTfR1 aptamers were evaluated for the ability to deliver siRNA in vitro using HeLa cells and an siRNA that targets HPRT1. All compounds were made using standard solid phase chemical synthesis using commercially available reagents.

In short, the aptamer sequences corresponding to core aptamer sequences in AP-00248, AP-00264 and AP-00385 were conjugated to the 3′ end of the sense stand of an anti-HPRT1 siRNA. Compounds based on the sequence of AP-00131 (SEQ ID NO: 59) served as a non-targeting negative control (Table 16). Compounds based on AP-00074 served as a positive control. AP-00074 (SEQ ID NO: 55) corresponds to known hTfR1 aptamer Waz (SEQ ID NO: 60) with a single point mutation and has equivalent hTfR1 binding characteristics to Waz.

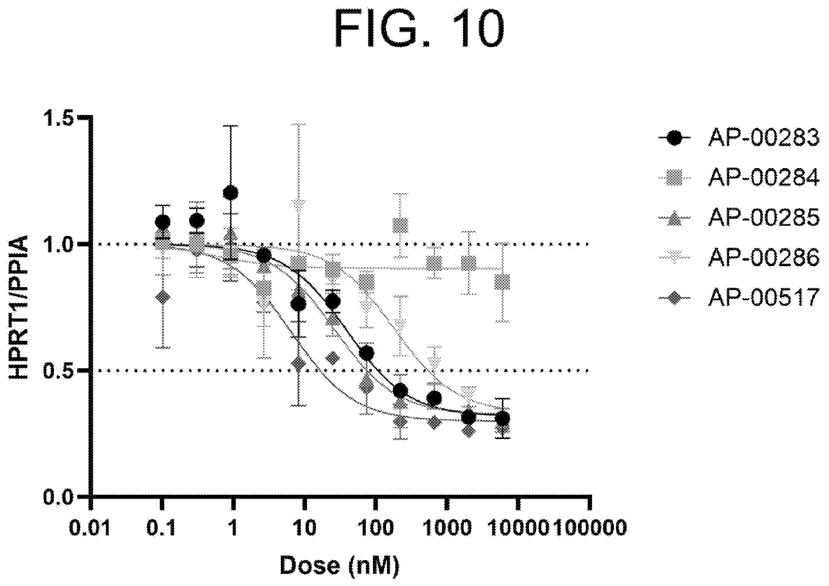

The compounds were combined with 3000 Hela cells in 100 μL media (DMEM+10% FBS) in a 96-well tissue culture plate. Cells were incubated at 37° C. and 5% CO₂ for 96 hr after which the cells were lysed (iScript, Bio-Rad). The recovered RNA was used directly for relative mRNA quantitation by qPCR and the change in expression of HPRT1 compared to that of PPIA. As shown in FIG. 10, hTfR targeting aptamers result in a dose dependent knockdown of HPRT1 mRNA, with the compound based on AP-00385 demonstrating the most potent activity (FIG. 10 and Table 16). Importantly, when similar experiments were performed using just the siRNA duplex (AA-05259) or the siRNA duplex conjugated to a non-functional aptamer sequence (AP-00284) no appreciable knockdown was observed (IC50>10 μM).

TABLE 16
Sequences of aptamers conjugated with siRNA

| Compound | Compound | siRNA sequence | Aptamer Sequence | Aptamer | IC50 |
|---|---|---|---|---|---|
| Name | components | (5′ to 3′) | (5′ to 3′) | SEQ ID NO: | (nM) |
| AP- | AP-00074- | AA-05259 | rGrGrGfUfUfCfUrAfCrG | 171 | 39 |
| 00283 | (Sp18)-siRNA | | AfUrArArAfCrGrGfUfUr | | |
| | | | ArAfUrGrAfUfCrArGfCf | | |
| | | | UfUrAfUrGrGfCfUrGrGf | | |
| | | | CrArGfUfUfCfCfC | | |
| AP- | AP-00131- | AA-05259 | GGCGUAGUGAUUAUGAAUCG | 59 | >10,000 |
| 00284 | (sp18)-siRNA | | UGUGCUAAUACACGCC | | |
| AP- | AP-00248- | AA-05259 | GCAACCCGGAGGGAUAGGAA | 41 | 26 |
| 00285 | (sp18)-siRNA | | UUAUCGGUUGC | | |
| AP- | AP-00264- | AA-05259 | CGCGGUGUGGAAAUGUAAAG | 45 | 182 |
| 00286 | (sp18)-siRNA | | AUCGUUGCCGCG | | |
| AP- | AP-00385- | AA-05259 | CGGACUUACGUAGAAAGGUA | 53 | 6.6 |
| 00517 | (sp18)-siRNA | | ACGAUCAUUGUGUCCG | | |
| AA- | siRNA (only) | fUCfCUfAUGAfCUG | N/A | N/A | >10,000 |
| 05259 | | UfAgfAUfUUfUAfU | | | |
| | | (SEQ ID NO: 61) | | | |
| | | pAfUAfAAfAUfCUf | | | |
| | | ACfAgfUCfAUfAgf | | | |
| | | GAfAU | | | |
| | | (SEQ ID NO: 62) | | | |

Where G is 2′F and A, C and U are 2′OMe modified RNA, rG and rA are 2′OH RNA, fA, fC and fU are 2′F RNA, g is G-2′Ome, p is a 5′ phosphate, (Sp18) is hexaethylene glycol, and / denotes a separation between two hybridized oligonucleotides.

### Example 7. In Vivo siRNA Delivery Using Anti-TfR Aptamers

Aptamer siRNA conjugates were evaluated for the ability to target and knock down gene expression in muscle tissue following systemic delivery using human transferrin receptor knock-in mice (hTfR1-mice). For these studies, in addition to assessing the activity of compounds AP-00285, AP-00286 and the control sequence AP-00284, we also generated PEGylated versions of these compounds bearing a 20 kDa PEG to slow renal clearance. PEG conjugates were generated via conjugation to the 5′ end of the aptamer using 20 kDa-PEG-NHS and a 5′ amine modified aptamer (C6NH2; Table 17).

TABLE 17
Aptamers conjugated with PEG

| Compound Name | Compound components |
|---|---|
| AP-00312 | AP-00284-20kPEG |
| AP-00313 | AP-00285-20k PEG |
| AP-00314 | AP-00286-20k PEG |

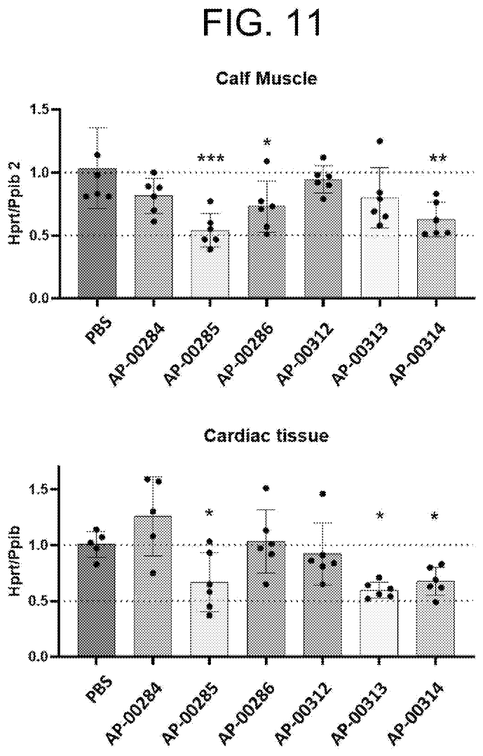

Aptamer-siRNA conjugates or PEGylated variants were injected into hTfR1-mice intravenously at 10 mg/kg. Three days later (72 hr) mice were euthanized, and RNA extracted from tissue using standard procedures. Recovered RNA was subsequently reverse transcribed and HPRT1 mRNA expression levels measured by qPCR. The house keeping gene, PPIB2 served as reference. As shown in FIG. 11, hTfR1-targeted aptamers (AP-00285, AP-00286, AP-00308, AP-00309) led to significant HPRT1 knockdown in both muscle and calf tissue. Importantly, animals treated with control aptamers (AP-00284 and AP-00307) failed to show any significant response.

### Example 8. In Vivo ASO Delivery Using Anti-hTfR1 Aptamers

Aptamer-ASO conjugates targeting the lncRNA Malat-1 were generated to evaluate the potential to knockdown target gene expression in muscle using RNAseH mediated degradation. In short, the aptamer sequences corresponding to core aptamer sequences in AP-00248, AP-00264 were conjugated to the 5′ end of the sense strand the Malat-1 ASO (AA-00593) using a hexaethylene glycol (Sp18) linker (Table 18). Compounds based on the sequence of AP-00131 served as a non-targeting negative control. To investigate the potential effect of PEGylation to slow renal clearance, we also generated a series of aptamer-ASO conjugates in which the parent compounds, AP-00340, AP-00341 and AP-00342 were hybridized to an oligonucleotide complementary to AA-00593 bearing a 5′ 20 kDa PEG (AA-00344, SEQ ID NO: 63). The PEG conjugate in AA-00344 was generated via conjugation to the 5′ end of the oligonucleotide using 20 kDa-PEG-NHS via a 5′ amine (C6NH2; Table 18).

TABLE 18
Sequences of aptamers conjugated with ASO and PEG

| | Compound | | | Complimentary |
|---|---|---|---|---|
| Compound | Components | Aptamer Sequence | ASO Sequence | Oligo Sequence |
| Name | (5′ to 3′) | (5′ to 3′) | (5′ to 3′) | (5′ to 3′) |
| AP-00340 | AP-00131- | GGCGUAGUGAUUAUGAAUCGUGUGCU | +G*+C*+A*T*T*5mdC* | N/A |
| | (Sp18)- | AAUACACGCC (SEQ ID NO: 59) | T*dA*dA*T*dA*dG*5m | |
| | AA-00593 | | dC*+A*+G*+5mC (SEQ | |
| | | | ID NO: 24) | |
| AP-00341 | AP-00248- | GCAACCCGGAGGGAUAGGAAUUAUCG | +G*+C*+A*T*T*5mdC* | N/A |
| | (Sp18)- | GUUGC (SEQ ID NO: 41) | T*dA*dA*T*dA*dG*5m | |
| | AA-00593 | | dC*+A*+G*+5mC (SEQ | |
| | | | ID NO: 24) | |
| AP-00342 | AP-00264- | CGCGGUGUGGAAAUGUAAAGAUCGUU | +G*+C*+A*T*T*5mdC* | N/A |
| | (Sp18)- | GCCGCG (SEQ ID NO:45) | T*dA*dA*T*dA*dG*5m | |
| | AA-00593 | | dC*+A*+G*+5mC (SEQ | |
| | | | ID NO: 24) | |
| AP-00350 | AP-00340 | GGCGUAGUGAUUAUGAAUCGUGUGCU | +G*+C*+A*T*T*5mdC* | 20 kDaPEG- |
| | AP-00344 | AAUACACGCC (SEQ ID NO: 59) | T*dA*dA*T*dA*dG*5m | mGCUdGdCdTd |
| | | | dC*+A*+G*+5mC (SEQ | AdTdTdAdGdA |
| | | | ID NO: 24) | dAUmGC (SEQ |
| | | | | ID NO: 63) |
| AP-00351 | AP-00341 | GCAACCCGGAGGGAUAGGAAUUAUCG | +G*+C*+A*T*T*5mdC* | 20 kDaPEG- |
| | AP-00344 | GUUGC (SEQ ID NO: 41) | T*dA*dA*T*dA*dG*5m | mGCUdGdCdTd |
| | | | dC*+A*+G*+5mC (SEQ | AdTdTdAdGdA |
| | | | ID NO: 24) | dAUmGC (SEQ |
| | | | | ID NO: 63) |
| AP-00352 | AP-00342 | CGCGGUGUGGAAAUGUAAAGAUCGUU | +G*+C*+A*T*T*5mdC* | 20 kDaPEG- |
| | AP-00344 | GCCGCG (SEQ ID NO: 45) | T*dA*dA*T*dA*dG*5m | mGCUdGdCdTd |
| | | | dC*+A*+G*+5mC (SEQ | AdTdTdAdGdA |
| | | | ID NO: 24) | dAUmGC (SEQ |
| | | | | ID NO: 63) |
| AA-00593 | ASO (only) | N/A | +G*+C*+A*T*T*5mdC* | N/A |
| | | | T*dA*dA*T*dA*dG*5m | |
| | | | dC*+A*+G*+5mC (SEQ | |
| | | | ID NO: 24) | |
| AP-00344 | PEG- | N/A | N/A | 20 kDaPEG- |
| | complimentary | | | mGCUdGdCdTd |
| | oligo-(only) | | | AdTdTdAdGdA |
| | | | | dAUmGC (SEQ |
| | | | | ID NO: 63) |

Where and G is 2′F; A, C and U are 2′OMe modified RNA; dA, dG, T are DNA, mG is G-2′OMe, 5mC is 5-methylcytidine and 5mdC is 5-methyl deoxycytidine; + denotes LNA; * is a phosphorothioate (PS) linkage and (Sp18) is hexethylene glycol

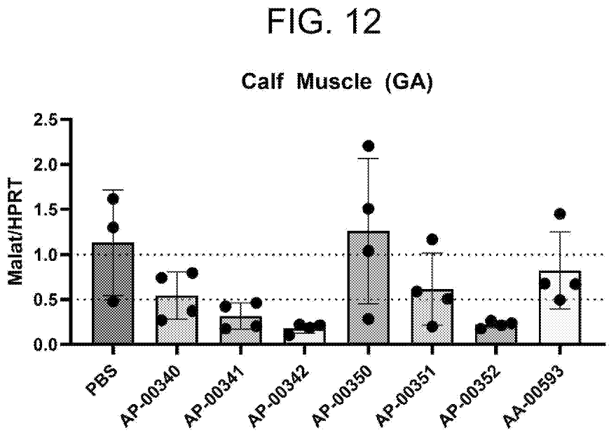

Aptamer-ASO conjugates (Table 18) were evaluated in hTfR1 knock-in mice. In short, animals were dosed weekly at 5 mg/kg for 4 weeks after which RNA was extracted from tissue using standard procedures and the expression level of Malat-1 RNA was determined by qPCR, with HPRT1 serving as reference. As shown in FIG. 12, hTfR1 targeted aptamers (AP-00341, AP-00342, AP-00351, AP-00352) resulted in knockdown of Malat-1 expression with less knockdown observed for animals treated with the non-targeted aptamer (AP-00340 and AP-00350) or the ASO alone (AA-00593). Interestingly, the combination of duplexation and PEGylation of the control sequence appeared to block non-specific uptake suggesting a method to minimize non-specific uptake.

### Example 9. In Vitro LNP/mRNA Delivery Using Anti-hTfR1 Aptamers

Anti-hTfR1 aptamers based on the core sequences of AP-00248 and AP-00385 were evaluated for the ability to enhance the delivery and expression of mRNA using aptamer targeted lipid nanoparticles (LNPs). Thiol modified aptamers were chemically synthesized using standard solid phase chemical synthesis using commercially available reagents (Table 19). Aptamer LNPs were generated by post-insertion of aptamer-DSPE conjugates. Thiol modified aptamers were reduced in 100 mM TEAA (triethylammonium acetate) buffer at pH 7.0 containing 10 mM TCEP by heating at 70 C.° for 3 minutes, followed by incubation at room temperate for 30 minutes. Reduced aptamers were desalted (Biospin6, Bio-Rad) after which 5 μM aptamer was combined with a 20-fold molar excess of DSPE-PEG(2000)-maleimide in PBS buffer supplemented with 2 mM EDTA. Reactions were allowed to proceed overnight at room temperature and subsequently quenched by the addition of 1 mM β-mercaptoethanol (BME) and used without additional purification.

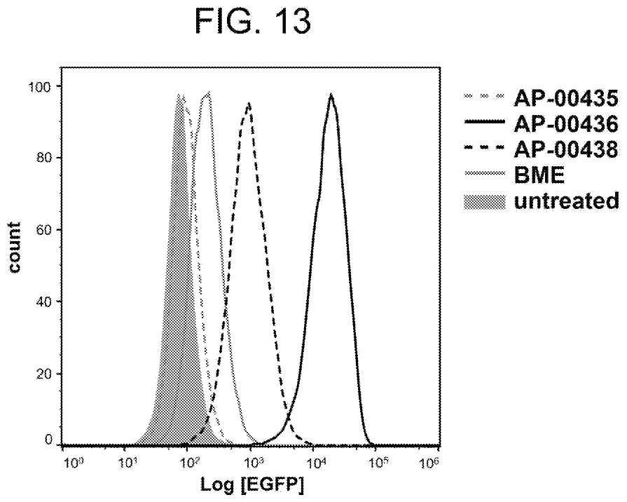

Targeted LNPs or controls were generated by post-insertion using commercially available LNPs composed of SM-102, DSPC, cholesterol and DMG-PEG2000 containing mRNA encoding the enhanced green fluorescent protein EGFP (Cat #PM-LNP-0020, ProMab). These commercially available LNPs have the ability to deliver mRNA into cells. LNP functionalization was achieved by incubating 2 μL of aptamer-lipid conjugate or a BME control (100 μM DSPE-PEG(2000)-maleimide quenched with 1 mM BME) with 4 μL of pre-formed LNPs (approx. 50 μg/mL encapsulated mRNA). The mixtures were incubated at 45 C.° for two hours, after which 5 μL of each mixture was added to ~50,000 Hela cells in 200 μL DMEM containing 10% FBS. Following overnight incubation (18 hr) the cells were briefly treated with trypsin, washed in FACS buffer and EGFP expression was evaluated by flow cytometry. As shown in FIG. 13, LNPs functionalized with anti-hTfR1 aptamers (AP-00436 and AP-00438) showed a significant enhancement in EGFP expression when compared with LNPs bearing BME-quenched DSPE-PEG(2000)-maleimide (BME; FIG. 13) and an even greater enhancement when compared to LNPs bearing a non-functional control aptamer (AP-00435). Thus, not only are anti-hTfR1 aptamers capable of enhancing the uptake and expression of mRNA cargoes, but their presence on the LNP surface provides a means to block non-specific uptake.

TABLE 19
Sequences of thiol modified aptamers

| Compound | Modifi- | Aptamer Sequence |
|---|---|---|
| Name | cation | (5′ to 3′) |
| AP-00435 | 5SS | GGCGUAGUGAUUAUGAAUCGU |
| | | GUGCUAAUACACGCC |
| | | (SEQ ID NO: 59) |
| AP-00436 | 5SS | UCGGUAGACUUACGUAGAAAG |
| | | GUAACGAUCAUUGUGUCGUAU |
| | | CGA (SEQ ID NO: 64) |
| AP-00438 | 5SS | GCAACCCGGAGGGAUAGGAAU |
| | | UAUCGGUUGC |
| | | (SEQ ID NO: 41) |

Where all compounds bear a 5′ C6 terminal thiol (5SS) and G is 2′F and A, C and U are 2′OMe modified RNA

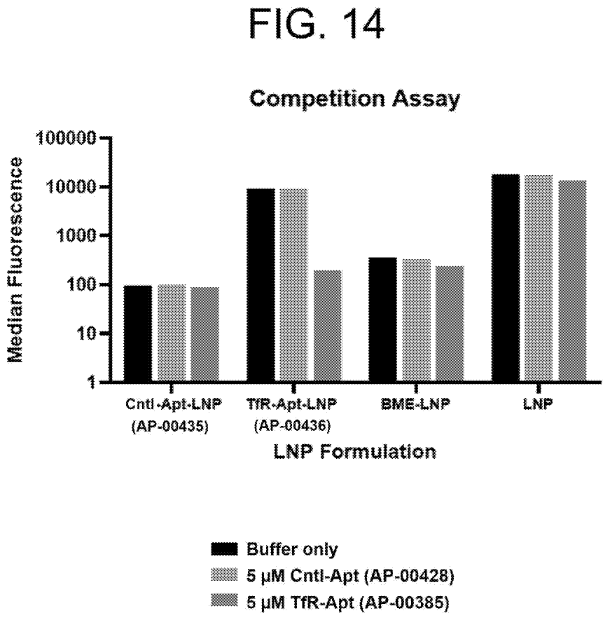

Competition experiments were performed to confirm the specificity of anti-hTfR1 uptake and EGFP expression. For these experiments, LNPs, BME-LNPs, Cntl-Apt-LNPs or TfR-Apt-LNPs were added to HeLa cells alone (buffer only) or in the presence of 5 μM Cntl-Apt (AP-000428) or TfR-Apt (AP-000385). AP-000428 corresponds to AP-131 with a 5′ amine (C6NH2). Following overnight incubation, cells were analyzed for EGFP expression by flow cytometry. As shown in FIG. 14, only the addition of free TfR-Apt blocked uptake and EGFP expression of TfR-Apt-LNPs.

### Example 10. Species Cross Reactivity of Anti-TfR Aptamers

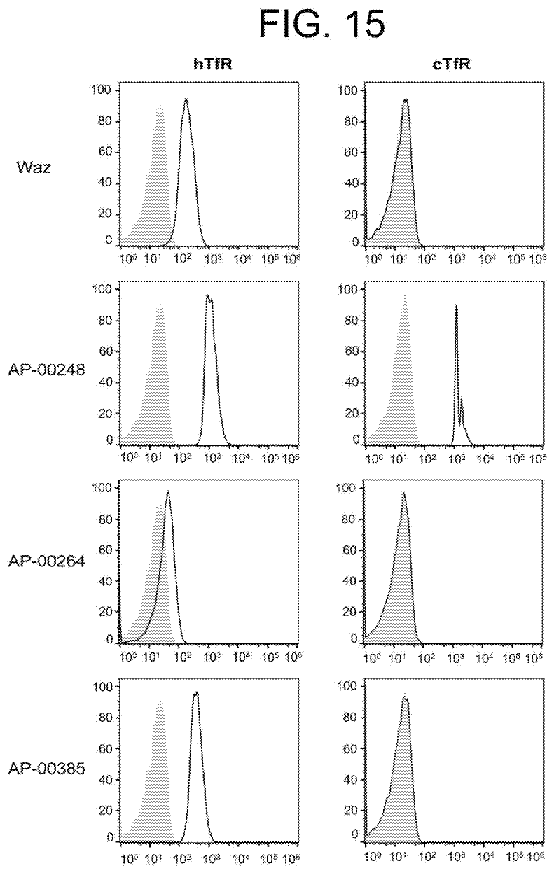

Species cross-reactivity was evaluated by flow cytometry using 5′ BP Fluor 647 labeled aptamers and recombinant His-tagged proteins immobilized on DYNABEADS® His-Tag Isolation and Pulldown beads. Amine modified aptamers were labelled as described above and flow cytometry assays were performed as described above. As shown in FIG. 15 and summarized in Table 20, under these conditions while all aptamers demonstrate binding to the human protein (hTfR), only AP-00248 demonstrates binding to protein from cynomolgus monkey (cTfR; Macaca fascicularis).

TABLE 20
Species cross-reactivity of aptamers
determined using flow cytometry

| Compound Name | SEQ ID NO | hTfR | cTfR |
|---|---|---|---|
| AP-00074 | 55 | ++ | − |
| AP-00248 | 41 | +++ | +++ |
| AP-00403 | 45 | + | − |
| AP-00385 | 53 | +++ | − |

+ >2-fold,
++ >10 fold,
+++ >50-fold over background,
− = no appreciable signal above background

### Example 11. Measurement of Aptamer Binding Affinity

Aptamer binding affinities were assessed using bio-layer interferometry (Gator® Prime, Gator Bio, Palo Alto, CA). In short, biotinylated avi-hTfR protein (Acro Biologicals) was immobilized on streptavidin probes and subsequently incubated with thermally equilibrated unlabelled aptamers in SB1T buffer. Binding constants were determined from a global fit of the data using the equation K_{D}=kₒₙ/k_{off}. Results are summarized in Table 21.

TABLE 21
Binding affinities of aptamers determined
using bio-layer interferometry

| Compound Name | SEQ ID NO | K_{D} (nM) |
|---|---|---|
| AP-00074 | 55 | 79 |
| AP-00248 | 41 | 138 |
| AP-00403 | 45 | 282 |
| AP-00385 | 53 | 9 |
| AP-00414 | 54 | 26 |

### Example 12: Aptamer Synthesis

Chemical synthesis of aptamers may be performed using standard solid phase nucleic acid synthesis chemistry, for example on an Dr. Oligo 48 (Biolytic, Fremont, CA). Phosphoramidite monomers may be purchased from ChemGenes (Wilmington, MA), Glen Research (Sterling, VA) or other sources. Inverted 3′-3′ dT columns (dT-5′-SynBase CPG) may be purchased from Link Technologies (Bellshill, United Kingdom). All other synthesis reagents may be purchased form Glen Research (Sterling, VA). Aptamers may be generated with a 3′ inverted dT residue for added serum stability and a 5′ Thiol-Modifier C6 S—S or 5′5′-Amino-Modifier C6 for later chemical conjugations. Aptamers may be synthesized with the final dimethoxytrityl group (5′ DMT) left on to facilitate purification. Following deprotection, using standard procedures, aptamers may be purified by reversed-phase high-performance liquid chromatography (HPLC), for example on a 10×50 mm Xbridge C18 column (Waters, Milford, MA) using a linear gradient of acetonitrile in 0.1 M triethylammonium acetate (TEAA) at pH 7.5 at 65° C. Following deprotection, using standard procedures, aptamers may be purified by trityl catch using a Poly-pak or GlenPak (Sterling, VA) column or cartridge. Following deprotection, using standard procedures, aptamers may be purified by ion exchange chromatography.

The identity of each synthesised aptamer can be confirmed by mass spectrometry. Aptamers may be analyzed by mass spectrometry (MS) using, for example, Waters SQD2, single quadruple, and the UPLC is Waters Aquity I-class. For MS analysis, aptamers may be resuspended at 200 μM in TEAA. 5 μL samples may be desalted using a linear gradient of MeOH in 400 mM HFIP/8 mM TEA using, for example, a Waters Xbridge C18 2.1×50 mm column.

### Example 13: Serum Stability Assay

Aptamer stability in serum may affect the ability of the aptamer to localize to tumors or other in vivo target sites. Therefore, it may be useful to determine the likely in vivo stability of the aptamers disclosed herein using serum, such as fresh mouse serum. To this end, a suitable serum stability assay is described below.

For serum stability assays, whole blood from C57BL6 mice may be collected via terminal cardiac stick and allowed to clot for 20 minutes at room temperature. Whole blood may be centrifuged with a fixed angle rotor at 2000×g for 15 minutes at 4° C. to separate and collect the serum. Labeled AF750-labeled aptamers may be prepared (for instance, as described in Example 17) as 30 μM stocks in 10 μL PBS, heated to 70° C. and allowed to incubate on the benchtop for 15 minutes prior to use. Samples may then be added to 90 μL of serum for a final concentration of 3 μM in 100 μL of 90% serum. At each time point, 10 μL of reaction may be removed to a 0.5 mL tube, and the RNA may be recovered by a methanol chloroform extraction as previously described (Semple S C, et al. (2010) Rational design of cationic lipids for siRNA delivery. Semple et al., Nat Biotechnol (2010), 28(2): 172-176). In short, samples diluted with 50 μL of PBS, 100 μL methanol, and 50 μL chloroform may be mixed after which an additional 50 μL of water and 50 μL of chloroform may be added, and the tube vortexed again. The samples may then be spun at 500×g for 20 minutes at 4° C. in a fixed angle rotor. The aqueous layer (containing the RNA) may be collected and stored at −20° C. until completion of all time points. Samples may be diluted 1:1 in RNA loading buffer (95% formamide, 10 mM EDTA, 0.05% bromophenol blue), and 4 μL loaded onto an 8%, 19:1 (acrylamide:bis-acrylamide), 7 M urea gel. Gels may be scanned on a LI-COR Odyssey scanner or similar to visualize full length labeled aptamer. Full length aptamer may be quantified in the LI-COR Imaging Suite Lite or similar and normalized to the hour zero recovered RNA. Rates of decay and serum half-lives may be determined using GraphPad Prism or similar.

In some embodiments, in this serum stability assay the aptamers disclosed herein display half-lives greater than 8 hours. In some embodiments, aptamers displaying the longest half-life display a half-life of greater than 10 hours, greater than 12 hours, greater than 18 hours or greater than 24 hours, and perhaps greater than 48 hours and greater than 96 hours.

### Example 14: Media Stability Assay

It may be useful to assess the stability of the aptamers in media to ensure that degradation of the aptamers during the course of an assay is not confounding the results of that assay, especially for assays that involve longer timepoints. A suitable media stability assay is described below.

Aptamers labeled with DyLight 650 may be diluted in DMEM with 10% FBS to a concentration of 1 μM. Diluted aptamers may then be incubated at 37° C. for 1 or 24 hours. Post incubation samples may be diluted 10-fold in a 7M urea loading dye containing 1% SDS and heat denatured by heating at 65° C. for 5 minutes. Analysis may be performed by denaturing (7M urea) gel electrophoresis on a 12% polyacrylamide gel which has been pre-run with heated TBE containing 0.1% SDS. Gels may be analyzed on a Bio-Rad ChemiDocXRS+ imaging system or similar. Band signal intensity corresponding to the full length aptamer may be quantified using Image Lab 6.0 or similar and normalized to samples in media diluted and heat inactivated in 7M urea dye without 37° C. incubation.

In some embodiments, the aptamers disclosed herein are stable when tested in this media stability assay, and show little to no degradation at 24, 48 or 96 hours (for example >95% stability at 24 hours, >95% stability at 48 hours, or >95% stability at 96 hours).

### Example 15: In Vivo NIR Imaging

Suitable assays for assessing the ability of the aptamers disclosed herein to bind the transferrin receptor in vivo are described below.

For this purpose, it is possible to utilize aptamers labeled with Alexa Fluor 750 (AF750) or similar, near infrared (NIR) imaging, and PC3-PSMA or 22Rv1 flank tumor models or similar. The PC3-PSMA cell line described in Kelly et al. (2021) was stably transfected to express PSMA and displays high expression of hTfR in culture when measured by flow cytometry (>10-fold staining as assessed by aptamer staining using flow cytometry). The parental PSMA cell line is well documented in xenograft models. 22Rv1 cells naturally express high levels of hTfR and readily form tumors. It is also possible to perform an immunohistochemical analysis of hTfR expression in tumors generated from both cell lines in athymic nude mice to confirm maintained expression of these target receptors. The use of these tumors, therefore, provides a means to test in vivo anti-hTfR aptamers identified via in vitro methods.

Mice may be injected subcutaneously with 2×10⁶ to 5×10⁶ cells 3-6 weeks prior to imaging. Tumor growth may be monitored, and animals may be used for imaging when the tumor is at least 0.5 cm but <1.5 cm in diameter (for example tumors may be grown to ~0.5-1.0 cm in diameter). Tumor bearing mice may be injected with 2 nmoles of AF750 labeled aptamer in PBS via tail vein injection. For these in vivo studies, it is possible to use IVIS imaging to look for aptamer localization to tumors following intravenous (tail vein) injection. In particular, the mice may be imaged on an IVIS Spectrum Imager or similar 3, 6, 12, and 24 h post injection. Tumors may be size matched prior to the experiment for comparison to control sequences (to minimize the potential for artifacts that may result from variation in vascularization). For these studies, the animals need not be blinded or randomized.

Some aptamers disclosed herein, when viewed 12 h post injection in the 22Rv1 model, both dorsally and laterally, demonstrate significant tumor staining.

Some aptamers disclosed herein, when viewed 12 h post injection in the PC3-PSMA tumor model, both dorsally and laterally, demonstrate significant tumor staining.

Some aptamers disclosed herein, when viewed 12 h post injection in the 22Rv1 model, both dorsally and laterally, demonstrate increased tumor staining relative to a known hTfR aptamer (such as the Waz, C2 or C2.min aptamer).

Some aptamers disclosed herein, when viewed 12 h post injection in the PC3-PSMA tumor model, both dorsally and laterally, demonstrate increased tumor staining relative to a known hTfR aptamer (such as the Waz, C2 or C2.min aptamer).

### Example 16: In Vivo NIR Imaging Using AP-00248 and AP-00264

Aptamers AP-00248 and AP-00264 were evaluated for the ability to target A431 flank tumours and epidermal cancer cell line that expresses hTfR1. For these studies, amine modified anti-TfR1 aptamers AP-00248 and AP-00264 or non-targeting control AP-00131 were labelled with IR 750 using an NHS ester and standard procedures (Table 22).

TABLE 22
IR-750-labelled compounds

| Compound | | Parent | Aptamer SEQ |
|---|---|---|---|
| Name | 5′ Dye | compound | ID NO |
| AP-00300 | IR 750 | AP-00131 | 59 |
| AP-00301 | IR 750 | AP-00248 | 41 |
| AP-00302 | IR 750 | AP-00264 | 45 |

Mice were injected subcutaneously with ~2×10⁶ cells in matrigel ~2-3 weeks prior to imaging. Tumor growth was monitored, and imaging was performed when the tumor size was ~0.45 mm³. Tumor bearing mice were injected with 4 nmoles of IR750 labelled aptamer in PBS via tail vein injection and imaged 1, 4, 8, 12, and 24 hr post dose using a Kodak, Bruker Xtreme imaging system.

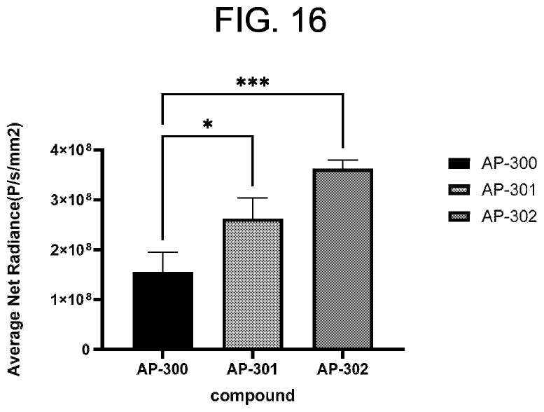

A comparison of the fluorescent signal observed in tumours extracted from mice treated with AP-00300, AP-00301 and AP-00302 24 hr post dose showed a significant increase in fluorescent signal for animals treated with hTfR1 targeting aptamers AP-00300, AP-00301 relative to animals treated with the non-targeting control AP-300 (FIG. 16).

### Example 17: Aptamer Dye Conjugation

It may be useful to conjugate a dye, such as a fluorescent dye, to the aptamers disclosed herein. A suitable aptamer dye conjugation protocol is explained below.

For this conjugation protocol, the aptamers may conveniently be synthesized with a 5′ disulfide modification, which can be used for subsequent conjugation to fluorophores. A 5′ DMT may be left on the aptamer following its synthesis to facilitate purification by reversed phase HPLC.

Dye conjugation may be performed in a manner similar to that previously described (Wilner S E, et al. (2012)). In short, individual aptamers may be suspended in 0.1 M TEAA with 10 mM TCEP and heated to 70° C. for 3 minutes. Reduction may be confirmed by analytical HPLC. Reduced aptamers may be desalted into PBS containing 2 mM EDTA using a Bio-Spin 6 desalting column (Bio-Rad) or similar. Maleimide activated dyes, for example DyLight 650 (Pierce, Rockford II) or similar for in vitro experiments or Alexa Fluor 750 (Invitrogen, Carlsbad CA) or similar for in vivo experiments, may be dissolved in DMSO and added to the reduced aptamers at a fivefold molar excess of dye. Dye reactions may be monitored by analytical HPLC and may be desalted into PBS with Bio-Spin 6 columns or similar to remove excess dye. Conjugation reactions routinely proceed to completion. The removal of free dye may be confirmed by analytical HPLC.

### Example 18: Computational Prediction of Aptamer Secondary Structure

The secondary structure of the aptamer sequences of compounds AP-248 (SEQ ID NO: 41), AP-264 (SEQ ID NO: 45) and AP-385 (SEQ ID NO: 53) were predicted using the UNAFold (Unified Nucleic Acid Folding and hybridization package, www.unafold.org), which uses the mfold software described in Zuker, 2003 for RNA folding predictions.

For all aptamer sequences, folding was performed with the percent suboptimality set to 50, which returns all predicted structures within 50% of the minimum free energy structure (MFE).

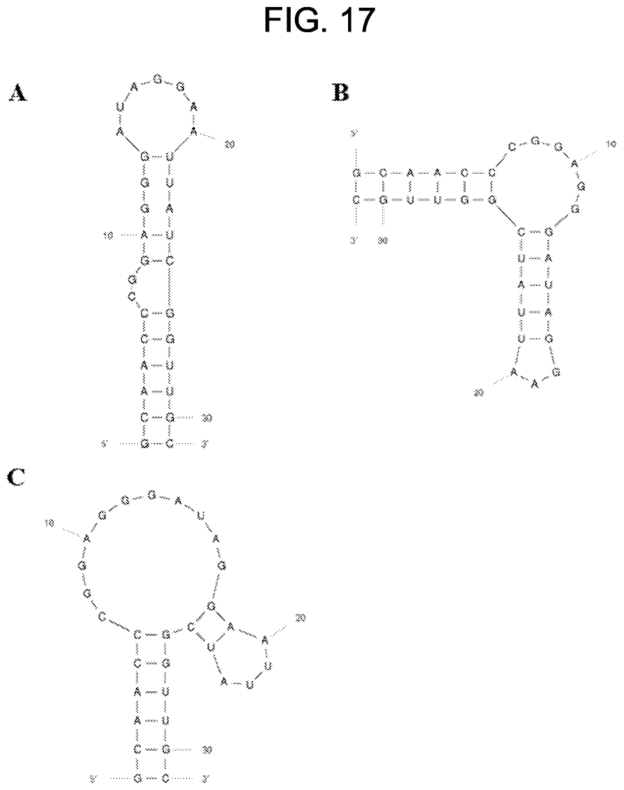

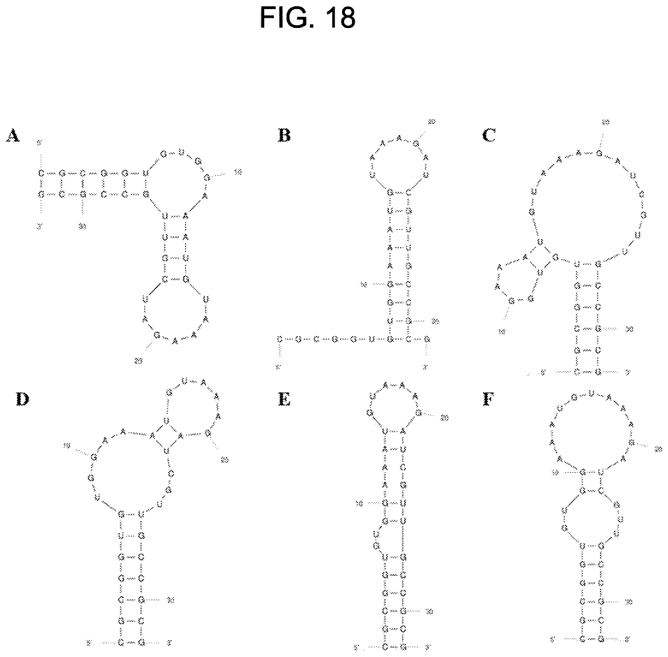

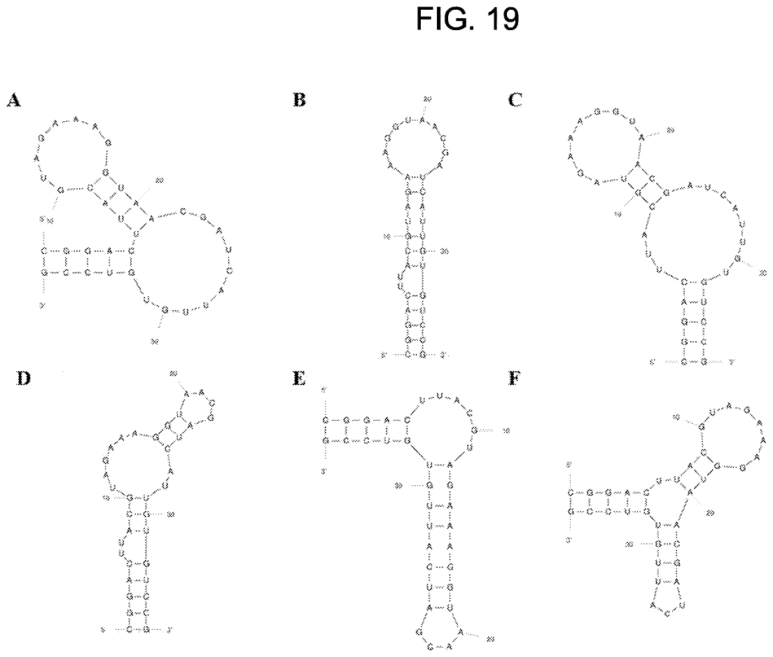

Three predicted structures were returned for AP-248 (FIG. 17), six predicted structures were returned for AP-264 (FIG. 18), and six predicted structures were returned for AP-385 (FIG. 19).

### Example 19: In Vivo ASO Delivery—DMPK (Study 1b)

Aptamer-ASO conjugates targeting the gene DMPK were generated to evaluate the potential to knockdown target gene expression in muscle using RNAseH mediated degradation. Aptamer-ASO conjugates corresponding to core aptamer sequences in AP-00248 (SEQ ID NO: 41) or AP-00385 (SEQ ID NO: 53) in which two additional base pairs were added to the closing stem (TA-00597 and TA-00596) were conjugated to the 3′ end of an ASO targeting DMPK (AA-05275 (SEQ ID NO: 74) or a non-RNAse H active variant, AA-05791 (SEQ ID NO: 75), using a hexaethylene glycol (Sp18) linker (Table 23). ASO only (AA-05275) served as a control.

TABLE 23
Aptamer-ASO conjugates

| | Compound | Aptamer | |
|---|---|---|---|
| Compound | Components | Sequence | ASO Sequence |
| Name | (5′ to 3′) | (5′ to 3′) | (5′ to 3′) |
| TA-00596 | AP-00385 | CmGCGGACUUACGUAGAAAGG | N/A |
| | variant | UAACGAUCAUUGUGUCCGCmG | |
| | | (SEQ ID NO: 72) | |
| TA-00597 | AP-00248 | CmGGCAACCCGGAGGGAUAGG | N/A |
| | variant | AAUUAUCGGUUGCCmG | |
| | | (SEQ ID NO: 73) | |
| AA-05275 | N/A | N/A | +A*+5mdC |
| | | | *+dA*dA*T*dA*dA*dA*T*dA*5mdC*5mdC |
| | | | *dG*+A*+G*+G (SEQ ID NO: 74) |
| AA-05791 | N/A | N/A | +A*+5mdC |
| | | | *dA*+A*T*dA*+A*dA*T*+A*5mdC*5mdC |
| | | | *+G*dA*+G*+G (SEQ ID NO: 75) |
| AP-00548 | AA-05275- | CmGCGGACUUACGUAGAAAGG | +A*+5mdC |
| | (Sp18)- | UAACGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| AP-00550 | AA-05275- | CmGGCAACCCGGAGGGAUAGG | +A*+5mdC |
| | (Sp18)- | AAUUAUCGGUUGCCmG | *+dA*dA*T*dA*dA*dA*T*dA*5mdC*5mdC |
| | TA-00597 | (SEQ ID NO: 73) | *dG*+A*+G*+G (SEQ ID NO: 74) |
| TA-00615 | AA-05791- | CmGCGGACUUACGUAGAAAGG | +A*+5mdC |
| | (Sp18)- | UAACGAUCAUUGUGUCCGCmG | *dA*+A*T*dA*+A*dA*T*+A*5mdC*5mdC |
| | TA-00596 | (SEQ ID NO: 72) | *+G*dA*+G*+G (SEQ ID NO: 75) |
| TA-00616 | AA-05791- | CmGGCAACCCGGAGGGAUAGG | +A*+5mdC |
| | (Sp18)- | AAUUAUCGGUUGCCmG | *dA*+A*T*dA*+A*dA*T*+A*5mdC*5mdC |
| | TA-00597 | (SEQ ID NO: 73) | *+G*dA*+G*+G (SEQ ID NO: 75) |

Where and G is 2′F; A, C and U are and mG are 2′OMe modified RNA; dA, dG, T are DNA,, 5mC is 5-methylcytidine and 5mdC is 5-methyl deoxycytidine; + denotes LNA; * is a phosphorothioate (PS) linkage and (Sp18) is hexethylene glycol

Compounds were evaluated in hTfR1 knock-in mice. AP-00548, AP-00550 and the ASO only (AA-05275) were all dosed at 1, 3, or 10 mg/kg. TA-00615 and TA-00616 were dosed at 3 mg/kg.

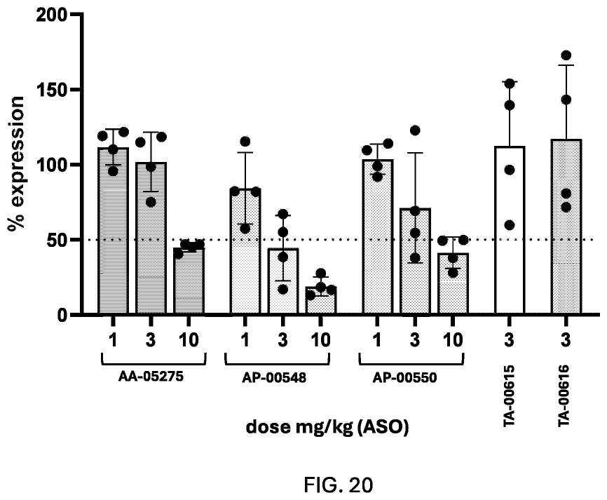

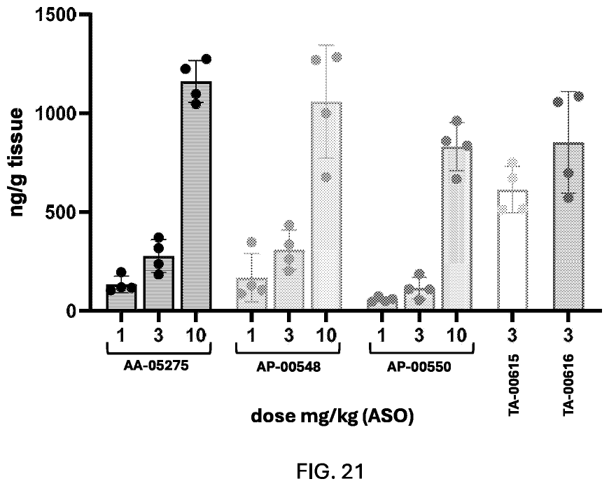

In short, animals were dosed on d1, d5 and d9 at 1, 3, or 10 mg/kg (ASO). On d14 following the initial dose, animals were euthanized, RNA was extracted from tissues using standard procedures and the expression level of DMPK RNA was determined by qPCR, with PPIB serving as reference. As shown in FIG. 20, hTfR1 targeted aptamers (AP-00548 and AP-00550) resulted in dose dependent knock down of DMPK expression in gastrocnemius calf muscle, with no knockdown observed for animals treated with the aptamer conjugates bearing a non-functional ASO (TA-00615 and TA-00616). Consistent with the literature, less knockdown was observed in animals treated with ASO only (AA-05275). Hybridization ELISA was used to evaluate tissue ASO concentrations. As shown in FIG. 21, tissue concentrations for AP-00548 and AA-05275 were roughly equivalent, whereas AP-00550 showed slightly lower tissue levels. These results suggest that targeting with TfR does not seem to significantly enhance bulk tissue concentration. The observed improvement in activity is therefore likely the result of specific localization within cells within the tissue. That is, TfR targeting improves cell uptake and improves cytosolic access leading to greater activity. Interestingly, conjugates bearing a non-functional ASO (TA-00615 and TA-00616) showed significantly higher tissue concentrations.

### Example 20: Effects of Lipid and Linker on Activity (Study 1c and Study 2)

The role of linker and lipid on the effect on TfR aptamer targeted ASO delivery was evaluated using a series of compounds based on the core aptamer AP-00385. All compounds (Table 24) were evaluated in hTfR1 knock-in mice. Compounds were dosed at 3 mg/kg on days 1, 5, and 9. Animals were euthanized on Day 16. RNA was extracted from tissues using standard procedures and the expression level of DMPK RNA was determined by qPCR, with PPIB serving as reference.

TABLE 24
Aptamer-ASO conjugates with alternative linkers and lipidation states

| | Compound | | |
|---|---|---|---|
| Compound | Components | Aptamer Sequence | |
| Name | (5′ to 3′) | (5′ to 3′) | ASO Sequence (5′ to 3′) |
| CR-AP-00548 | AA-05275- | CmGCGGACUUACGUAGAAAGGUAA | +A*+5mdC |
| | (Sp18)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00598 | L-AA-05275- | CmGCGGACUUACGUAGAAAGGUAA | +A*+5mdC |
| | (Sp18)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00599 | AA-05275- | CmGCGGACUUACGUAGAAAGGUAA | +A*+5mdC |
| | (C6)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00600 | L-AA-05275- | CmGCGGACUUACGUAGAAAGGUAA | +A*+5mdC |
| | (C6)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00601 | AA-05275- | CmGCGGACUUACGUAGAAAGGUAA | +A*+5mdC |
| | (C12)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00602 | L-AA-05275- | CmGCGGACUUACGUAGAAAGGUAA | +A*+5mdC |
| | (C12)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00603 | AA-05275- | CmGCGGACUUACGUAGAAAGGUAA | +A*+5mdC |
| | (TTT)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00604 | L-AA-05275- | CmGCGGACUUACGUAGAAAGGUAA | +A*+5mdC |
| | (TTT)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00718 | L-AA-05275- | CmGCGGACUUACGUAGAAAGGUAA | +A*+5mdC |
| | (T)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-TA-00694 | C12(OH)-AA- | CmGCGGACUUACGUAGAAAGGUAA | +A*+5mdC |
| | 05275-(Sp18)- | CGAUCAUUGUGUCCGCmG | *+dA*dA*T*dA*dA*dA*T*dA*5mdC*5mdC |
| | TA-00596 | (SEQ ID NO: 72) | *dG*+A*+G*+G (SEQ ID NO: 74) |
| CR-AP-00546 | AA-05275- | GGCGTAGTGATTATGAATCGTGTG | +A*+5mdC |
| | (Sp18)- | CTAATACACGCC | *+dA*dA*T*dA*dA*dA*T*dA*5mdC*5mdC |
| | AP-00131 | (SEQ ID NO: 172) | *dG*+A*+G*+G (SEQ ID NO: 74) |
| CR-TA-00698 | L-AA-05275- | GGCGTAGTGATTATGAATCGTGTG | +A*+5mdC |
| | (Sp18)- | CTAATACACGCC | *+dA*dA*T*dA*dA*dA*T*dA*5mdC*5mdC |
| | AP-00131 | (SEQ ID NO: 172) | *dG*+A*+G*+G (SEQ ID NO: 74) |

Where and G is 2′F; A, C and U and mG are 2′OMe modified RNA; dA, dG, T are DNA, 5mC is 5-methylcytidine and 5mdC is 5-methyl deoxycytidine; + denotes LNA; * is a phosphorothioate (PS) linkage, (Sp18) is hexethylene glycol, (C6) is 1, 6-heaxanediol, (C12) is 1, 12-dodecanediol, (C12OH) is a 5′ terminal 1, 12-dodecanediol, L is a palmityl-6-aminohexanol

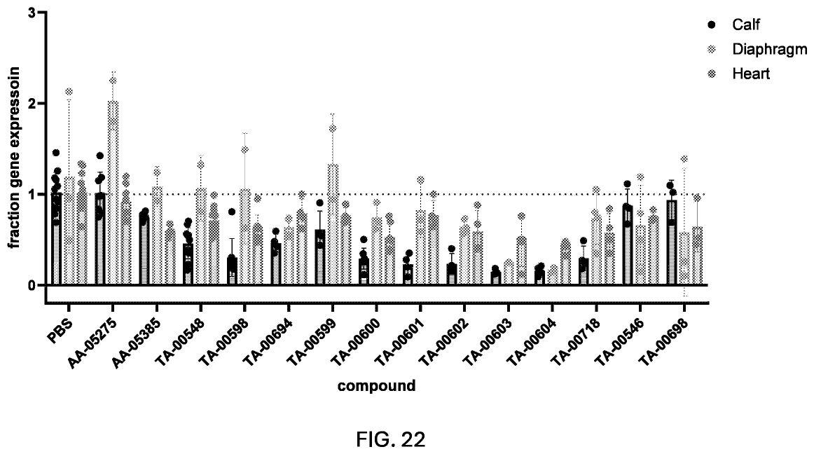

As shown in FIG. 22, the identity of the linker and the presence of lipid had varied effects on compound activity in muscle with the best performing linkers, TTT and C12 showing comparable knockdown levels regardless of lipidation state (TA-00601 v. TA-00602, TA-00603 v. TA-00604). Consistent with the literature, lipidation of the ASO alone resulted in a modest improvement in activity over the ASO alone. Lipidation did not significantly enhance the activity of the non-targeted control aptamer ASO conjugate (CR-AP-00546, CR-TA-00698).

### Example 21: Dose Response (Study 5)

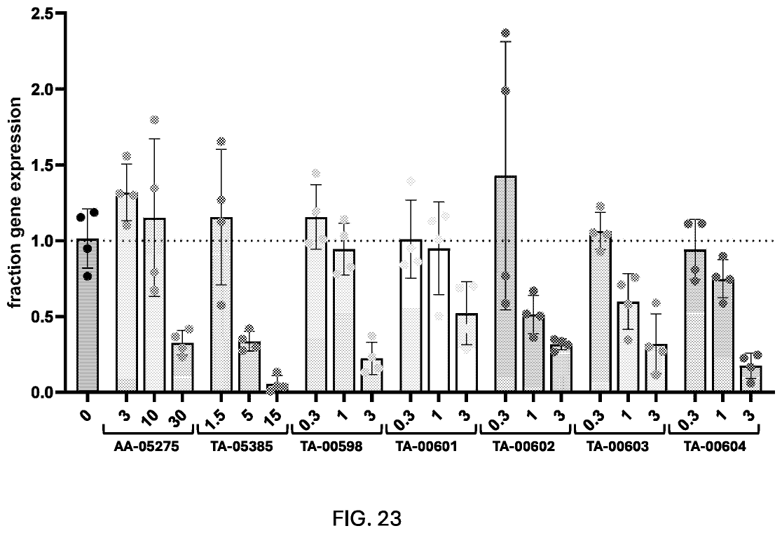

Compounds TA-00598, TA-00601, TA-00602, TA-00603 and TA-00604 were evaluated in hTfR1 knock-in mice and compared with ASO only (AA-05275) or lipidated ASO (AA-05385). Compounds were dosed as indicated in Table 25. All compounds were evaluated in hTfR1 knock-in mice and dosed on days 1, 5, and 9. Animals were euthanized on Day 16. RNA was extracted from tissues using standard procedures and the expression level of DMPK RNA was determined by qPCR, with PPIB serving as reference. As shown in FIG. 23, aptamer targeting resulted in a significant improvement of target gene knockdown vs non-targeted ASO or lipidated ASO.

TABLE 25
Dosing of select compounds

| Compound Name | Dose range (mg/kg) |
|---|---|
| AA-05275 | 3, 10, 30 |
| AA-05385 | 1.5, 5, 15 |
| TA-00598 | 0.3, 1, 3 |
| TA-00601 | 0.3, 1, 3 |
| TA-00602 | 0.3, 1, 3 |
| TA-00603 | 0.3, 1, 3 |
| TA-00604 | 0.3, 1, 3 |

Dose mass based on ASO component only

### Example 22: Duration of Action (Study 4)

Compounds TA-00598, TA-00601, TA-00602, TA-00603 and TA-00604 were evaluated in hTfR1 knock-in mice and compared with lipidated ASO (AA-05385). Compounds were dosed as indicated in Table 26.

TABLE 26
Administration and dosing of select compounds

| Compound Name | Dose range (mg/kg) | route |
|---|---|---|
| AA-05385 | 3 | iv |
| TA-00598 | 3 | iv |
| TA-00601 | 3 | iv, sc |
| TA-00602 | 3 | iv, sc |
| TA-00603 | 3 | iv, sc |
| TA-00604 | 3 | iv, sc |

Dose mass based on ASO component only

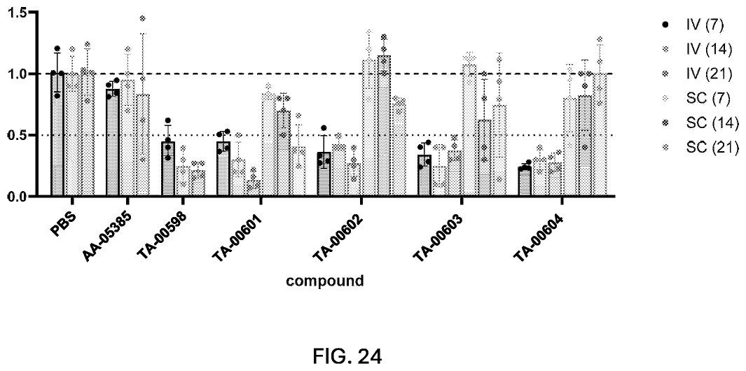

All compounds were evaluated in hTfR1 knock-in mice and dosed either via intravenous (iv) or subcutaneous (sc) administration on days 1, 5, and 9. Animal groups were euthanized on Day 16, 23 and 30 corresponding to 7, 14 and 21 days post last dose. RNA was extracted from tissues using standard procedures and the expression level of DMPK RNA was determined by qPCR, with PPIB serving as reference. As shown in FIG. 24, aptamer targeting resulted in a significant improvement of target gene knockdown vs non-targeted ASO or lipidated ASO. For animals treated by iv delivery, target knockdown was observed at the earliest time point (7 days post dose) and extended out to the last time point, 21 days post last dose. In some instances, knockdown activity appears to continue to increase over the sample period (TA-00598 and TA-00601). For animals treated by subcutaneous delivery, less activity was observed in all treatment groups at the earliest timepoints, with a progressive increase in target gene knockdown vs. time post dose for TA-00601, TA-00602 and to a lesser extent TA-00603. For TA-00601 at 21 days post last dose, a knockdown efficiency of ~60% was observed, rivalling that observed following iv dosing in other treatment groups.

### Example 23: Degenerate 385 Selection

In order to better understand the sequence and structure-function relationships within AP-00385, we performed a degenerate selection based on the sequence of AP-00385 using a synthetic library doped at 70% (Table 27).

TABLE 27
Library sequence and sequence
of oligos used to amplify the library

| | Sequence (5′ to 3′) |
|---|---|
| Library sequence | GGGAGATTCCGCAGCTAGTGTT |
| (Total library | CGGACTTACGTAGAAAGGTAAC |
| length: 81 bases) | GATCATTGTGTCCGTGCCTCTT |
| | CCTAACGGTAACCAA |
| | (SEQ ID NO: 167) |
| 385.F | AGGGAGATTCCGCAGCTAGTGT |
| | (SEQ ID NO: 168) |
| 385.R | TTGGTTACCGTTAGGAAGAGGC |
| | (SEQ ID NO: 169) |

Where G, A, T and C are deoxyribonucleotides and the 385.F primer also includes a 5′ phage polymerase promoter. Bolded positions indicate the degenerate library where the identity of each position is either 70% the base indicated and 10% the remaining 3 nucleotides or 85% the base indicated and 5% the remaining 3 nucleotides.

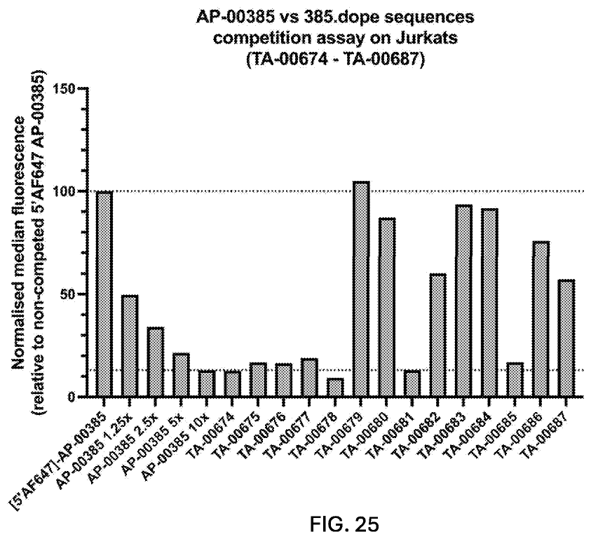

Briefly, using 50:50 mixture of fully backbone modified fGmH RNA libraries, we initiated a selection targeting hTfR1 immobilized on DYNABEADS Dynabeads™ MyOne™ Streptavidin C1 isolation and Pulldown beads (Thermofisher). The amount of target protein, input RNA, blocking agents and washing conditions varied between rounds. Following an initial 3 rounds of selection the libraries were sequenced. Individual compounds were synthesized chemically and assayed for binding activity against Jurkat cells using a flow-based competition assay. In short, unlabelled aptamer clones were premixed with 400 nM AF647-labeled AP-μ00385 in a 10-fold molar excess. Mixtures were added to Jurkat cells in full culture media supplemented with 1 mg/ml ssDNA as a blocking agent and 1 mg/ml Transferrin to investigate competition binding in the presence of the natural ligand. Following a 1 hr incubation at 37° C. the cells were washed 3× with FACS binding buffer and subsequently assayed by flow cytometry. Dead cells were eliminated by inclusion of DAPI the final resuspension buffer. As shown in FIG. 25, under these conditions, when unlabelled AP-00385 is used as a competitor, increasing concentrations results in a loss of AF647-labeled AP-000385 binding signal. The results of this competition binding assay are summarized in Table 28.

TABLE 28
Summary results from competition binding analysis

| | | | Competition |
|---|---|---|---|
| CRID | Alias | Sequence (5′ to 3′) | Assay Result |
| TA-00674 | MW_385d_ | CGGGCTTACGTAGAAAGGTAACGATCATTGTGTCCG | + |
| | R3b_2 | (SEQ ID NO: 78) | |
| TA-00675 | MW_385d_ | CGGTCTTACGTAGAAAGGTAACGATCATTGTGTCCG | − |
| | R3b_3 | (SEQ ID NO: 79) | |
| TA-00676 | MW_385d_ | CGGACTTACGTAGAAAGGAAACGATCATTGTGTCCG | − |
| | R3b_4 | (SEQ ID NO: 80) | |
| TA-00677 | MW_385d_ | CGGACTTACGTCGAAAGGTAACGATCATTGTGTCCG | − |
| | R3b_13 | (SEQ ID NO: 81) | |
| TA-00678 | MW_385d_ | CGGTCTTACGTAGAAAGGTAACGATCATTGTGGCCG | ++ |
| | R3b_16 | (SEQ ID NO: 82) | |
| TA-00679 | MW_385d_ | CGGACTTACGTAGAAAGGTAACGATCATTGT | −− |
| | R3b_22 | (SEQ ID NO: 83) | |
| TA-00680 | MW_385d_ | CGGACTTACGTAGAGAGGTAACGATCATTGTGTCCG | −− |
| | R3b_25 | (SEQ ID NO: 84) | |
| TA-00681 | MW_385d_ | CGGTCTTACGTAGAAAGGTAACGATCATTGTGACCG | + |
| | R3b_36 | (SEQ ID NO: 85) | |
| TA-00682 | MW_385d_ | CGGACTTACGTAGAAAGGCAACGATCATTGTGTCCG | − |
| | R3b_45 | (SEQ ID NO: 86) | |
| TA-00683 | MW_385d_ | CGGACTTACGTAGAAAGGTAACGATCATTTGTGTCCG | −− |
| | R3b_64 | (SEQ ID NO: 87) | |
| TA-00684 | MW_385d_ | CGGACTT_CGTAGAAAGGTAACGATCATTGTGTCCG | −− |
| | R3b_75 | (SEQ ID NO: 88) | |
| TA-00685 | MW_385d_ | CGGGCTTACGTAGAAAGGAAACGATCATTGTGTCCG | − |
| | 2 + 4 | (SEQ ID NO: 89) | |
| TA-00686 | MW_385_ | CGGACTTTACGTAGAAAGGTAACGATCATTGTGTCCG | −− |
| | UUUbulge | (SEQ ID NO: 90) | |
| TA-00687 | MW_385_ | CGGACT_ACGTAGAAAGGTAACGATCATTGTGTCCG | − |
| | Ubulge | (SEQ ID NO: 91) | |

−− = minimal binding/competition versus AP-00385 on Jurkats
− = slightly weakened binding/decreased competition versus AP-00385 on Jurkats
+ = equivalent binding/competition versus AP-00385 on Jurkats
++ = improved binding/increased competition versus AP-00385 on Jurkats

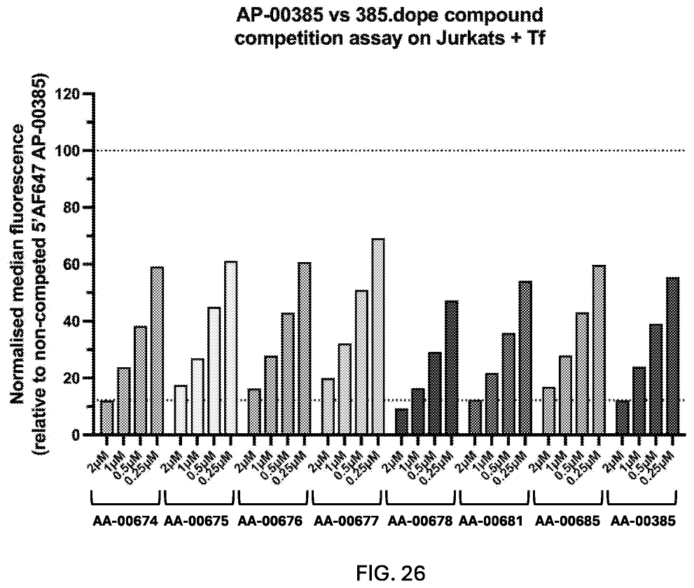

As a follow-up, selected compounds were assessed over a range of concentrations, 10×, 5×, 2.5× and 1.25×, with labelled aptamer again at 400 nM. Results shown in FIG. 26 are consistent with those in Table 28 and suggest the addition of the T:G at position 4 in TA-00678 results in a modest improvement in binding affinity.

### Example 24: Anti-TfR Aptamer Mediated In Vivo Delivery to the Brain

Transferrin receptor (TfR) is a bona fide target for the delivery of molecular cargoes to the brain. Here, we assess the ability of TfR aptamers to mediate delivery of siRNA to the brain.

Briefly, hTfR knock-in mice are treated with TA-00596 linked to siRNA that targets the house keeping gene HPRT1. The aptamer-siRNA conjugation is made by conjugating the 3′ end of the aptamer and the 5′ end of the siRNA sense strand using an SP18 linker. The aptamer-sense strand conjugate is made as a single compound and is subsequently allowed to hybridize to the antisense strand. A PEGylated version of the compound is generated by conjugating an NHS-activated 20 kDa PEG molecule to the 5′ end of the aptamer. The PEG is conjugated to the aptamer using a 5′ hexylamine modification.

hTfR knock-in mice (n=3) are dosed iv with 1 mg/kg or 10 mg/kg compound based on the weight of the siRNA. At 3 and 14 days post-dose, animals are euthanized and whole brain homogenate is extracted and assessed for target gene knockdown. Thirty percent (30%) knockdown is observed in animals in the 14-day group treated with 10 mg/kg compound.

It will be appreciated that numerous variations and/or modifications may be made to the features of the invention described and exemplified herein without departing from the spirit or scope of the invention. The features of the invention described and exemplified herein are, therefore, to be considered in all respects as illustrative and not restrictive.

All publications cited herein are incorporated by reference in their entirety.

1. A compound comprising an aptamer, wherein the aptamer comprises:
a. a core having a sequence of:
(SEQ ID NO: 100)
5′-YGURGAAAKGUAAMGAUCRUUG-3′,
(SEQ ID NO: 101)
5′-UGURGAAAKGUAAMGAUCGUUG-3′,
(SEQ ID NO: 102)
5′-CCCGGWGGGAUAGGAOUUAUCGG-3′,
or
a sequence having at least 95% identity to 105, 108, 111, 114, 117, 120, 123, 126, 129, 132, 135, 138, 141, 144, 147, 150, 153, 156, 159, 162, or 165, wherein
A is a nucleoside comprising an adenine (A) nucleobase,
C is a nucleoside comprising a cytosine (C) nucleobase,
G is a nucleoside comprising a guanine (G) nucleobase,
U is a nucleoside comprising a uracil (U) nucleobase,
Y is a nucleoside comprising a C or U nucleobase,
R is a nucleoside comprising an A or G nucleobase,
K is a nucleoside comprising a G or U nucleobase,
M is a nucleoside comprising an A or C nucleobase,
W is a nucleoside comprising an A or U nucleobase, and
O is a nucleoside comprising an A nucleobase or an absent nucleoside;
b. a 5′ stem region (X) linked to 5′-end of the core and comprising nucleosides comprising A, C, G or U nucleobases; and
c. a 3′ stem region (Z) linked to 3′-end of the core and comprising nucleosides comprising A, C, G or U nucleobases,
wherein the 5′ stem region, the 3′ stem region and optionally, one or more nucleosides in the core can interact to form a stem structure.

2. The compound of claim 1, wherein
the 5′ stem region (X) and the 3′ stem region (Z) have each independently a sequence of 4 to 25 linked nucleosides comprising A, C, G or U nucleobases.

3. (canceled)

4. The compound of claim 1, wherein one or more pyrimidine nucleosides of the aptamer comprise;
a 2′-fluoro-modified ribose sugar moiety; or
a 2′-O-methyl-modified ribose sugar moiety.

5. The compound of claim 1, wherein one or more purine nucleosides of the aptamer comprise:
a 2′-fluoro-modified ribose sugar moiety; or
a 2′-O-methyl-modified ribose sugar moiety.

6.-9. (canceled)

10. The compound of claim 1, wherein one or more guanosine nucleosides of the aptamer comprise a 2′-fluoro-modified ribose sugar moiety.

11. (canceled)

12. The compound of claim 1, wherein one or more adenosine, cytosine or uracil nucleosides of the aptamer comprise a 2′-O-methyl-modified ribose sugar moiety.

13. (canceled)

14. The compound of claim 1, wherein all of the nucleosides of the aptamer are connected by phosphodiester internucleoside linkages.

15. The compound of claim 1, wherein the aptamer comprises a core having at least 98%, 99% or 100% sequence identity to SEQ ID NO: 105, 108, 111, 114, 117, 120, 123, 126, 129, 132, 135, 138, 141, 144, 147, 150, 153, 156, 159, 162, or 165.

16. The compound of claim 1, wherein the aptamer comprises a 5′-stem region (X) and 3′-stem region (Z) pair selected from
i. SEQ ID NO: 104 and SEQ ID NO: 106;
ii. SEQ ID NO: 107 and SEQ ID NO: 109;
iii. SEQ ID NO: 110 and SEQ ID NO:112;
iv. SEQ ID NO:113 and SEQ ID NO: 115;
v. SEQ ID NO: 116 and SEQ ID NO: 118;
vi. SEQ ID NO:119 and SEQ ID NO: 121;
vii. SEQ ID NO: 122 and SEQ ID NO:124;
viii. SEQ ID NO: 125 and SEQ ID NO: 127;
ix. SEQ ID NO:128 and SEQ ID NO: 130;
x. SEQ ID NO: 131 and SEQ ID NO: 133;
xi. SEQ ID NO: 134 and SEQ ID NO: 136;
xii. SEQ ID NO: 137 and SEQ ID NO: 139;
xiii. SEQ ID NO: 140 and SEQ ID NO: 142;
xiv. SEQ ID NO: 143 and SEQ ID NO: 145;
xv. SEQ ID NO: 146 and SEQ ID NO: 148;
xvi. SEQ ID NO: 149 and SEQ ID NO: 151;
xvii. SEQ ID NO: 152 and SEQ ID NO: 154;
xviii. SEQ ID NO: 155 and SEQ ID NO: 157;
xix. SEQ ID NO: 158 and SEQ ID NO: 160;
xx. SEQ ID NO: 161 and SEQ ID NO: 163; and
xxi SEQ ID NO: 165 and SEQ ID NO: 166.

17. The compound of claim 1, wherein the 5′-stem region comprises a sequence of X₁U(2-8)X₂, wherein:
a. U(2-8) comprises 2 to 8 U nucleobases,
b. X₁ comprises one or more nucleosides comprising A, C, G or U nucleobases, and
c. X₂ comprises one or more nucleosides comprising A, C, G or U nucleobases, wherein U(2-8) can form a bulge in the stem structure.

18. The compound of claim 1, wherein the aptamer comprises a sequence having at least 95%, 98%, 99% or 100% sequence identity to SEQ ID NO: 71, 53, 56, 48, 49, 50, 54, 57, 58, 43, 45, 26, 27, 37, 44, 52, 38, 41, 15, 16, 17, 19, 39, or 40.

19. The compound of claim 1, wherein the aptamer comprises a core having the sequence of SEQ ID: 105.

20. The compound of claim 19, wherein the aptamer comprises:
(a) the sequence of linked nucleosides:
5′-CGGACUUACGUAGAAAGGUAACGAUCAUUGUGUCCG-3′ (SEQ ID NO: 53), wherein the nucleobases of the underlined nucleosides interact to form a stem structure in a computationally predicted secondary structure of the folded aptamer, wherein:
all guanines (G) comprise a 2-fluoro-modified ribose sugar moiety, and
all adenosines (A), cytosines (C) and uracils (U) of the aptamer comprise a 2′-O-methyl-modified ribose sugar moiety; or
(b) the sequence of the linked nucleosides:
(SEQ ID NO: 72)
5′-CmGCGGACUUACGUAGAAAGGUAACGAUCAUUGUGUCCGCmG-3′,
wherein:
all guanines (G) comprise a 2-fluoro-modified ribose sugar moiety (2′F), and
all adenines (A), cytosines (C), uracils (U) and methylated guanines (mG) comprise a 2′-O-methyl-modified ribose sugar moiety (2′OMe).

21. (canceled)

22. The compound of claim 20, wherein the aptamer comprises the sequence of
(SEQ ID NO: 72)
5′-CmGCGGACUUACGUAGAAAGGUAACGAUCAUUGUGUCCGCmG-3′

23.-28. (canceled)

29. The compound of claim 1, wherein the aptamer comprises any one of sequences selected from SEQ ID NO: 78-91.

30.-37. (canceled)

38. The compound of claim 1, wherein the aptamer (i) is capable of binding to hTfR1 on the surface of human cells to be subsequently internalized by the human cells; and (ii) is capable of binding to transferrin receptor 1 (TfR1) on the surface of cells of a non-human primate (NHP) to be subsequently internalized by the NHP cells.

39.-44. (canceled)

45. The compound of claim 1, wherein the compound is an aptamer conjugate in which the aptamer is conjugated to one or more of a detectable label, an antisense compound, a small organic molecule of less than 2000 Daltons (Da), polyethylene glycol (PEG), or a nanoparticle.

46.-49. (canceled)

50. The compound of claim 45, wherein the aptamer is conjugated to an antisense compound or to a lipid nanoparticle.

51. The compound of claim 50, wherein the antisense compound is a single-stranded antisense oligonucleotide or an siRNA.

52.-56. (canceled)

57. The compound of claim 50, wherein the lipid nanoparticle comprises a nucleic acid, optionally wherein the nucleic acid is a single-stranded antisense oligonucleotide, an siRNA or an mRNA.

58.-60. (canceled)

61. The compound of claim 1, comprising, from the 5′ to 3′ end or from 3′ to 5′ end:
a. a payload, a linker, and an aptamer;
b. an aptamer, a linker and a payload;
c. a payload, a linker, an aptamer and a ligand;
d. a payload, a linker, a ligand and an aptamer;
e. a ligand, a linker, an aptamer and a payload;
f. a ligand, a linker, a payload and an aptamer;
g. an aptamer, a linker, a ligand and a payload; or
h. an aptamer, a linker, a payload and a ligand.

62.-70. (canceled)

71. A pharmaceutical composition comprising the compound of claim 1 and one or more pharmaceutically acceptable carriers or diluents.

72. (canceled)

73. A method of treating a disease or condition in a mammalian subject in need thereof, the method comprising administering a therapeutically effective amount of the pharmaceutical composition of claim 71 to the subject in need thereof and thereby treating the disease or condition.

74. The or the method of claim 73, wherein the disease or condition is a disease or condition of the brain, skeletal muscle, or cardiac muscle.

75.-94. (canceled)

95. A method of delivering a payload into target tissue or target cells of a mammalian subject, wherein the target cells of the mammalian subject express a mammalian transferrin receptor on their surface, the method comprising administering a compound of claim 1 to the mammalian subject, optionally wherein following administration of the compound to the mammalian subject the aptamer binds to the transferrin receptor on the surface of the target mammalian cells and the compound is subsequently internalized by the target mammalian cells.

96.-107. (canceled)

108. The compound of claim 1, wherein the aptamer comprises
(a) the core having the sequence of CGUAGAAAGGUAACGAUCAUUG (SEQ ID NO: 105);
(b) the 5′-stem region (X) having the sequence of CGGACUUA (SEQ ID NO: 104); and
(c) the 3′-stem region (Z) having the sequence of UGUCCG (SEQ ID NO: 106);
wherein all of the nucleosides of the aptamer are connected by phosphodiester internucleoside linkages, and wherein the underlined nucleosides interact to form a stem structure.

109. The compound of claim 108, wherein the aptamer comprises the sequence of linked nucleosides 5′-CmGCGGACUUACGUAGAAAGGUAACGAUCAUUGUGUCCGCmG-3′ (SEQ ID NO: 72), wherein:
all guanines (G) comprise a 2-fluoro-modified ribose sugar moiety (2′F), and
all adenines (A), cytosines (C), uracils (U) and methylated guanines (mG) comprise a 2′-O-methyl-modified ribose sugar moiety (2′OMe).


SEQUENCE LISTING (172 sequences)

SEQ ID NO: 1 - DNA, 94 bp, synthetic construct
  source 1..94: mol_type = other DNA
  misc_difference 29..73: note = a, c, t, g, unknown or other
gggagaatgccgtagcagcgagaatagtnnnnnnnnnnnnnnnnnnnnnnnnnnnnnnnnnnnnnnnnnnnnnttatacatggcttgagccggc

SEQ ID NO: 2 - DNA, 27 bp, synthetic construct
  source 1..27: mol_type = other DNA
gggagaatgccgtagcagcgagaatag

SEQ ID NO: 3 - DNA, 20 bp, synthetic construct
  source 1..20: mol_type = other DNA
gccggctcaagccatgtata

SEQ ID NO: 4 - RNA, 94 nt, synthetic construct
  source 1..94: mol_type = other RNA
gggagaatgccgtagcagcgagaatagtatacgcaacccggtgggataggattatcggttgcataagccattgttatacatggcttgagccggc

SEQ ID NO: 5 - RNA, 94 nt, synthetic construct
  source 1..94: mol_type = other RNA
gggagaatgccgtagcagcgagaatagtcgacagacccggagggataggattatcggtcttgagatgcaaactttatacatggcttgagccggc

SEQ ID NO: 6 - RNA, 94 nt, synthetic construct
  source 1..94: mol_type = other RNA
gggagaatgccgtagcagcgagaatagtcccatagatcccggagggataggaattatcggatctaaacgtctgttatacatggcttgagccggc

SEQ ID NO: 7 - RNA, 94 nt, synthetic construct
  source 1..94: mol_type = other RNA
gggagaatgccgtagcagcgagaatagtccaatggtcaaccgaacagcggatagcattggggtagcggtcggtttatacatggcttgagccggc

SEQ ID NO: 8 - RNA, 94 nt, synthetic construct
  source 1..94: mol_type = other RNA
gggagaatgccgtagcagcgagaatagttgggcgtctcgccattatgacccggtgggataggattatcggtctttatacatggcttgagccggc

SEQ ID NO: 9 - RNA, 94 nt, synthetic construct
  source 1..94: mol_type = other RNA
gggagaatgccgtagcagcgagaatagtccggacagtccacaggtaggaaaagttgtggctaaattgtcaacgttatacatggcttgagccggc

SEQ ID NO: 10 - RNA, 94 nt, synthetic construct
  source 1..94: mol_type = other RNA
gggagaatgccgtagcagcgagaatagttctgggcgtacacagaaacctgcggactgtacgctaagatgtcggttatacatggcttgagccggc

SEQ ID NO: 11 - RNA, 94 nt, synthetic construct
  source 1..94: mol_type = other RNA
gggagaatgccgtagcagcgagaatagtggtagcaaaccggtctgatatgttgttatgcgtttgaccctgttgttatacatggcttgagccggc

SEQ ID NO: 12 - RNA, 94 nt, synthetic construct
  source 1..94: mol_type = other RNA
gggagaatgccgtagcagcgagaatagtacccgcagagtccggagtgtgtaggttagcgggcatggtatgcagttatacatggcttgagccggc

SEQ ID NO: 13 - RNA, 94 nt, synthetic construct
  source 1..94: mol_type = other RNA
gggagaatgccgtagcagcgagaatagcgcggtgtggaaatgtaaagatcgttgccgcgaacgacggttgtttttatacatggcttgagccggc

SEQ ID NO: 14 - RNA, 95 nt, synthetic construct
  source 1..95: mol_type = other RNA
gggagaatgccgtagcagcgagaatagcgcggtgtggaaatgtaaagatcgttgccgcgaacgacggttgttttttatacatggcttgagccggc

SEQ ID NO: 15 - RNA, 47 nt, synthetic construct
  source 1..47: mol_type = other RNA
tatacgcaacccggtgggataggattatcggttgcataagccattgt

SEQ ID NO: 16 - RNA, 47 nt, synthetic construct
  source 1..47: mol_type = other RNA
tcgacagacccggagggataggattatcggtcttgagatgcaaactt

SEQ ID NO: 17 - RNA, 47 nt, synthetic construct
  source 1..47: mol_type = other RNA
tcccatagatcccggagggataggaattatcggatctaaacgtctgt

SEQ ID NO: 18 - RNA, 47 nt, synthetic construct
  source 1..47: mol_type = other RNA
tccaatggtcaaccgaacagcggatagcattggggtagcggtcggtt

SEQ ID NO: 19 - RNA, 47 nt, synthetic construct
  source 1..47: mol_type = other RNA
ttgggcgtctcgccattatgacccggtgggataggattatcggtctt

SEQ ID NO: 20 - RNA, 47 nt, synthetic construct
  source 1..47: mol_type = other RNA
tccggacagtccacaggtaggaaaagttgtggctaaattgtcaacgt

SEQ ID NO: 21 - RNA, 47 nt, synthetic construct
  source 1..47: mol_type = other RNA
ttctgggcgtacacagaaacctgcggactgtacgctaagatgtcggt

SEQ ID NO: 22 - RNA, 47 nt, synthetic construct
  source 1..47: mol_type = other RNA
tggtagcaaaccggtctgatatgttgttatgcgtttgaccctgttgt

SEQ ID NO: 23 - RNA, 47 nt, synthetic construct
  source 1..47: mol_type = other RNA
tacccgcagagtccggagtgtgtaggttagcgggcatggtatgcagt

SEQ ID NO: 24 - DNA, 16 bp, synthetic construct
  source 1..16: mol_type = other DNA
  modified_base 1: mod_base = OTHER; note = Locked nucleic acid with phosphorothioate linkage
  modified_base 2: mod_base = OTHER; note = Locked nucleic acid with phosphorothioate linkage
  modified_base 3: mod_base = OTHER; note = Locked nucleic acid with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = 5-methyl deoxycytidine with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = Nucleotide with phosphorothioate linkage
  modified_base 12: mod_base = OTHER; note = Nucleotide with phosphorothioate linkage
  modified_base 13: mod_base = OTHER; note = 5-methyl deoxycytidine with phosphorothioate linkage
  modified_base 14: mod_base = OTHER; note = Locked nucleic acid with phosphorothioate linkage
  modified_base 15: mod_base = OTHER; note = Locked nucleic acid with phosphorothioate linkage
  modified_base 16: mod_base = OTHER; note = Locked 5-methylcytidine
gcattctaatagcagc

SEQ ID NO: 25 - RNA, 96 nt, synthetic construct
  source 1..96: mol_type = other RNA
gggagaatgccgtagcagcgagaatagcgcggtgtggaaatgtaaagatcgttgccgcgaacgacggttgtttttttatacatggcttgagccggc

SEQ ID NO: 26 - RNA, 47 nt, synthetic construct
  source 1..47: mol_type = other RNA
cgcggtgtggaaatgtaaagatcgttgccgcgaacgacggttgtttt

SEQ ID NO: 27 - RNA, 49 nt, synthetic construct
  source 1..49: mol_type = other RNA
cgcggtgtggaaatgtaaagatcgttgccgcgaacgacggttgtttttt

SEQ ID NO: 28 - DNA, 76 bp, synthetic construct
  source 1..76: mol_type = other DNA
  misc_difference 21..55: note = a, c, t, g, unknown or other
gggagagtcggtagcagtctnnnnnnnnnnnnnnnnnnnnnnnnnnnnnnnnnnntctatgtggaaatggcgctgt

SEQ ID NO: 29 - DNA, 19 bp, synthetic construct
  source 1..19: mol_type = other DNA
gggagagtcggtagcagtc

SEQ ID NO: 30 - DNA, 20 bp, synthetic construct
  source 1..20: mol_type = other DNA
acagcgccatttccacatag

SEQ ID NO: 31 - RNA, 76 nt, synthetic construct
  source 1..76: mol_type = other RNA
gggagagtcggtagcagtctgtagaaaggtaacgatcgttggactcactgtaatctctatgtggaaatggcgctgt

SEQ ID NO: 32 - RNA, 76 nt, synthetic construct
  source 1..76: mol_type = other RNA
gggagagtcggtagcagtcttggtggtcccagggttgttggttgctcgtcccgtatctatgtggaaatggcgctgt

SEQ ID NO: 33 - RNA, 76 nt, synthetic construct
  source 1..76: mol_type = other RNA
gggagagtcggtagcagtctgcaggtgcctgcggctgttgcttgctttgtcgttttctatgtggaaatggcgctgt

SEQ ID NO: 34 - RNA, 76 nt, synthetic construct
  source 1..76: mol_type = other RNA
gggagagtcggtagcagtcttggccgcagggttgttgtttgctcttccgtagttctctatgtggaaatggcgctgt

SEQ ID NO: 35 - RNA, 76 nt, synthetic construct
  source 1..76: mol_type = other RNA
gggagagtcggtagcagtctttggcgaaatcaggttctgttgtttgctttacccgtctatgtggaaatggcgctgt

SEQ ID NO: 36 - RNA, 76 nt, synthetic construct
  source 1..76: mol_type = other RNA
gggagagtcggtagcagtctgctgcaggtaactgttgtcctgttgtttgttctcctctatgtggaaatggcgctgt

SEQ ID NO: 37 - RNA, 56 nt, synthetic construct
  source 1..56: mol_type = other RNA
gggagagtcggtagcagtctgtagaaaggtaacgatcgttggactcactgtaatct

SEQ ID NO: 38 - RNA, 33 nt, synthetic construct
  source 1..33: mol_type = other RNA
  misc_difference 1..5: note = This region may encompass 2-5 nucleotides
  misc_difference 21: note = a or absent
  misc_difference 29..33: note = This region may encompass 2-5 nucleotides
nnnnncccggwgggatagganttatcggnnnnn

SEQ ID NO: 39 - RNA, 30 nt, synthetic construct
  source 1..30: mol_type = other RNA
gcaacccggtgggataggattatcggttgc

SEQ ID NO: 40 - RNA, 30 nt, synthetic construct
  source 1..30: mol_type = other RNA
gcaacccggagggataggattatcggttgc

SEQ ID NO: 41 - RNA, 31 nt, synthetic construct
  source 1..31: mol_type = other RNA
gcaacccggagggataggaattatcggttgc

SEQ ID NO: 42 - RNA, 28 nt, synthetic construct
  source 1..28: mol_type = other RNA
gcgcccggtgggataggattatcggcgc

SEQ ID NO: 43 - RNA, 32 nt, synthetic construct
  source 1..32: mol_type = other RNA
  misc_difference 1..5: note = This region may encompass 4-5 nucleotides
  misc_difference 28..32: note = This region may encompass 4-5 nucleotides
nnnnntgtrgaaakgtaamgatcgttgnnnnn

SEQ ID NO: 44 - RNA, 39 nt, synthetic construct
  source 1..39: mol_type = other RNA
cgcggtgtggaaatgtaaagatcgttgccgcgaacgacg

SEQ ID NO: 45 - RNA, 32 nt, synthetic construct
  source 1..32: mol_type = other RNA
cgcggtgtggaaatgtaaagatcgttgccgcg

SEQ ID NO: 46 - DNA, 95 bp, synthetic construct
  source 1..95: mol_type = other DNA
gggagcgtagctagtatccgggagagtcggtagcagtctgtagaaaggtaacgatcgttggactcactgtaatctctatgtggaaatggcgctgt

SEQ ID NO: 47 - DNA, 19 bp, synthetic construct
  source 1..19: mol_type = other DNA
gggagcgtagctagtatcc

SEQ ID NO: 48 - RNA, 56 nt, synthetic construct
  source 1..56: mol_type = other RNA
cggagactcggtagacttacgtagaaaggtaacgatcattgtgtcgtatcgattcg

SEQ ID NO: 49 - RNA, 56 nt, synthetic construct
  source 1..56: mol_type = other RNA
ggcagagtcggtgggcttacgtagaaaggtaacgatcattgtgctcactgtcatgt

SEQ ID NO: 50 - RNA, 56 nt, synthetic construct
  source 1..56: mol_type = other RNA
tgaaaaattagtcggcttacgtagaaaggtaacgatcattgtgctgactgaagtct

SEQ ID NO: 51 - RNA, 57 nt, synthetic construct
  source 1..57: mol_type = other RNA
agttccttacgtagctcttgacgtaaaagatcacgattgtgggagtgacaggaaccg

SEQ ID NO: 52 - RNA, 56 nt, synthetic construct
  source 1..56: mol_type = other RNA
cgtagacgatttaccggtccgtagaaatgtaaagatcgttggactcggtgtctact

SEQ ID NO: 53 - RNA, 36 nt, synthetic construct
  source 1..36: mol_type = other RNA
cggacttacgtagaaaggtaacgatcattgtgtccg

SEQ ID NO: 54 - RNA, 38 nt, synthetic construct
  source 1..38: mol_type = other RNA
cgcgacttacgtagaaaggtaacgatcattgtgtcgcg

SEQ ID NO: 55 - RNA, 48 nt, synthetic construct
  source 1..48: mol_type = other RNA
gggttctacgataaacggttaatgatcagcttatggctggcagttccc

SEQ ID NO: 56 - RNA, 44 nt, synthetic construct
  source 1..44: mol_type = other RNA
  misc_difference 1..9: note = This region may encompass 3-9 nucleotides
  misc_difference 36..44: note = This region may encompass 3-9 nucleotides
nnnnnnnnnttnygtrgaaakgtaamgatcrttgnnnnnnnnnn

SEQ ID NO: 57 - RNA, 32 nt, synthetic construct
  source 1..32: mol_type = other RNA
gacttacgtagaaaggtaacgatcattgtgtc

SEQ ID NO: 58 - RNA, 38 nt, synthetic construct
  source 1..38: mol_type = other RNA
  misc_difference 1..3: note = This region may encompass 2-3 nucleotides
  misc_difference 36..38: note = This region may encompass 2-3 nucleotides
sssgacttacgtagaaaggtaacgatcattgtgtcsss

SEQ ID NO: 59 - RNA, 36 nt, synthetic construct
  source 1..36: mol_type = other RNA
ggcgtagtgattatgaatcgtgtgctaatacacgcc

SEQ ID NO: 60 - RNA, 48 nt, synthetic construct
  source 1..48: mol_type = other RNA
gggttctacgataaacggttaatgaccagcttatggctggcagttccc

SEQ ID NO: 61 - RNA, 21 nt, synthetic construct
  source 1..21: mol_type = other RNA
  modified_base 1: mod_base = OTHER; note = 2'-fluoro modified nucleotide
  modified_base 3: mod_base = OTHER; note = 2'-fluoro modified nucleotide
  modified_base 5: mod_base = OTHER; note = 2'-fluoro modified nucleotide
  modified_base 7: mod_base = OTHER; note = 2'-fluoro modified nucleotide
  modified_base 9: mod_base = OTHER; note = 2'-fluoro modified nucleotide
  modified_base 11: mod_base = OTHER; note = 2'-fluoro modified nucleotide
  modified_base 13: mod_base = OTHER; note = 2'-fluoro modified nucleotide
  modified_base 15: mod_base = OTHER; note = 2'-fluoro modified nucleotide
  modified_base 17: mod_base = OTHER; note = 2'-fluoro modified nucleotide
  modified_base 19: mod_base = OTHER; note = 2'-fluoro modified nucleotide
  modified_base 21: mod_base = OTHER; note = 2'-fluoro modified nucleotide
tcctatgactgtagattttat

SEQ ID NO: 62 - RNA, 23 nt, synthetic construct
  source 1..23: mol_type = other RNA
  modified_base 1: mod_base = OTHER; note = 5'-phosphate modified nucleotide
  modified_base 2: mod_base = OTHER; note = 2'-fluoro modified nucleotide
  modified_base 4: mod_base = OTHER; note = 2'-fluoro modified nucleotide
  modified_base 6: mod_base = OTHER; note = 2'-fluoro modified nucleotide
  modified_base 8: mod_base = OTHER; note = 2'-fluoro modified nucleotide
  modified_base 10: mod_base = OTHER; note = 2'-fluoro modified nucleotide
  modified_base 12: mod_base = OTHER; note = 2'-fluoro modified nucleotide
  modified_base 14: mod_base = OTHER; note = 2'-fluoro modified nucleotide
  modified_base 16: mod_base = OTHER; note = 2'-fluoro modified nucleotide
  modified_base 18: mod_base = OTHER; note = 2'-fluoro modified nucleotide
  modified_base 20: mod_base = OTHER; note = 2'-fluoro modified nucleotide
  modified_base 22: mod_base = OTHER; note = 2'-fluoro modified nucleotide
ataaaatctacagtcataggaat

SEQ ID NO: 63 - DNA, 16 bp, synthetic construct
  source 1..16: mol_type = other DNA
  modified_base 1: mod_base = OTHER; note = 2'-O-methyl modified nucleotide attached to 20 kilodalton polyethylene glycol
  modified_base 15: mod_base = OTHER; note = 2'-O-methyl modified nucleotide
  misc_feature 1..3: note = RNA
  misc_feature 4..13: note = DNA
  misc_feature 14..16: note = RNA
gctgctattagaatgc

SEQ ID NO: 64 - RNA, 45 nt, synthetic construct
  source 1..45: mol_type = other RNA
tcggtagacttacgtagaaaggtaacgatcattgtgtcgtatcga

SEQ ID NO: 65 - RNA, 50 nt, synthetic construct
  source 1..50: mol_type = other RNA
tcaaacatctcacagatcaatccaagggacctcgttaaaggacgactccc

SEQ ID NO: 66 - RNA, 43 nt, synthetic construct
  source 1..43: mol_type = other RNA
gggggatcaatccaagggacccggaaacgctcccttacacccc

SEQ ID NO: 67 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 68 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 69 - protein, 760 aa, Macaca fascicularis
MMDQARSAFSNLFGGEPLSYTRFSLARQVDGDNSHVEMKLGVDEEENTDNNTKANGTKPKRCGGNICYGTIAVIIFFLIGFMIGYLGYCKGVEPKTECERLAGTESPAREEPEEDFPAAPRLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 70 - protein, 698 aa, Macaca fascicularis
MRLAVGALLACAVLGLCLAVPEKSVRWCAVSEHEATKCQSFRDHMKSVLPSDGPSVACVKKASHLDCIRAIAANEADAVTLDAGLVYDAYLAPNNLKPVVAEFYGSKEDPQTFYYAVAVVKKESDFQMNQLRGRKSCHTGLGRSAGWNIPIGLLYCDLPEPRKPLEKAVANFFSGSCVPCADGTDFPQLCQLCPGCGCSTLNQYFSYSGAFKCLKDGAGDVAFVKHSTIFENLANKADRDQYELLCLDNTRKSVDEYKDCHLARVPSHTVVARSVGGKEDLIWELLNQAQEHFGKDKSKEFQLFSSPHGKDLLFKDSAHGFLKVPPRMDAKMYLGYEYITAIRNLREGTCPEAPTDECKPVKWCALSHHERLKCDEWSVNSAGKIECESAETTEDCIAKIMNGEADAMSLDGGFVYIAGMCGLVPVLAENYNTPNPNCENTAEEGYFAVAVVKSSSDLTWDNLKGKKSCHTAVGRTAGWNIPMGLLYSKINHCRFDEFFSEGCAPGSEKNSSLCKLCMGPSPNLCEPNNKEGYYGYTGAFRCLVEKGDVAFVKHQTVPQNTGGNNPDAWAKNLKEEDYELLCLDGSRKSVQEPANCHLARAPNHAVVARKDKADCVQTLLLDQQRMFGSSVTDCSSNFCLFESKTKDLLFRDDTVCLAKLHDRNTYEKYLGEEYVKAVTNLRKCSTSPLLEACTFHRA

SEQ ID NO: 71 - RNA, 46 nt, synthetic construct
  source 1..46: mol_type = other RNA
  misc_difference 1..12: note = This region may encompass 4-12 nucleotides
  misc_difference 35..46: note = This region may encompass 4-12 nucleotides
nnnnnnnnnnnnygtrgaaakgtaamgatcrttgnnnnnnnnnnnn

SEQ ID NO: 72 - RNA, 40 nt, synthetic construct
  source 1..40: mol_type = other RNA
  modified_base 2: mod_base = OTHER; note = 2'-O-methyl modified nucleotide
  modified_base 40: mod_base = OTHER; note = 2'-O-methyl modified nucleotide
cgcggacttacgtagaaaggtaacgatcattgtgtccgcg

SEQ ID NO: 73 - RNA, 35 nt, synthetic construct
  source 1..35: mol_type = other RNA
  modified_base 2: mod_base = OTHER; note = 2'-O-methyl modified nucleotide
  modified_base 35: mod_base = OTHER; note = 2'-O-methyl modified nucleotide
cggcaacccggagggataggaattatcggttgccg

SEQ ID NO: 74 - DNA, 16 bp, synthetic construct
  source 1..16: mol_type = other DNA
  modified_base 1: mod_base = OTHER; note = Locked nucleic acid with phosphorothioate linkage
  modified_base 2: mod_base = OTHER; note = Locked 5-methyl deoxycytidine with phosphorothioate linkage
  modified_base 3: mod_base = OTHER; note = Locked nucleic acid with phosphorothioate linkage
  modified_base 4: mod_base = OTHER; note = Nucleotide with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Nucleotide with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Nucleotide with phosphorothioate linkage
  modified_base 11: mod_base = OTHER; note = 5-methyl deoxycytidine with phosphorothioate linkage
  modified_base 12: mod_base = OTHER; note = 5-methyl deoxycytidine with phosphorothioate linkage
  modified_base 13: mod_base = OTHER; note = Nucleotide with phosphorothioate linkage
  modified_base 14: mod_base = OTHER; note = Locked nucleic acid with phosphorothioate linkage
  modified_base 15: mod_base = OTHER; note = Locked nucleic acid with phosphorothioate linkage
  modified_base 16: mod_base = OTHER; note = Locked nucleic acid
acaataaataccgagg

SEQ ID NO: 75 - DNA, 16 bp, synthetic construct
  source 1..16: mol_type = other DNA
  modified_base 1: mod_base = OTHER; note = Locked nucleic acid with phosphorothioate linkage
  modified_base 2: mod_base = OTHER; note = Locked 5-methyl deoxycytidine with phosphorothioate linkage
  modified_base 3: mod_base = OTHER; note = Nucleotide with phosphorothioate linkage
  modified_base 4: mod_base = OTHER; note = Locked nucleic acid with phosphorothioate linkage
  modified_base 5: mod_base = OTHER; note = Nucleotide with phosphorothioate linkage
  modified_base 6: mod_base = OTHER; note = Nucleotide with phosphorothioate linkage
  modified_base 7: mod_base = OTHER; note = Locked nucleic acid with phosphorothioate linkage
  modified_base 8: mod_base = OTHER; note = Nucleotide with phosphorothioate linkage
  modified_base 9: mod_base = OTHER; note = Nucleotide with phosphorothioate linkage
  modified_base 10: mod_base = OTHER; note = Locked nucleic acid with phosphorothioate linkage
  modified_base 11: mod_base = OTHER; note = 5-methyl deoxycytidine with phosphorothioate linkage
  modified_base 12: mod_base = OTHER; note = 5-methyl deoxycytidine with phosphorothioate linkage
  modified_base 13: mod_base = OTHER; note = Locked nucleic acid with phosphorothioate linkage
  modified_base 14: mod_base = OTHER; note = Nucleotide with phosphorothioate linkage
  modified_base 15: mod_base = OTHER; note = Locked nucleic acid with phosphorothioate linkage
  modified_base 16: mod_base = OTHER; note = Locked nucleic acid
acaataaataccgagg

SEQ ID NO: 76-77 - skipped (2 entries)

SEQ ID NO: 78 - DNA, 36 bp, synthetic construct
  source 1..36: mol_type = other DNA
cgggcttacgtagaaaggtaacgatcattgtgtccg

SEQ ID NO: 79 - DNA, 36 bp, synthetic construct
  source 1..36: mol_type = other DNA
cggtcttacgtagaaaggtaacgatcattgtgtccg

SEQ ID NO: 80 - DNA, 36 bp, synthetic construct
  source 1..36: mol_type = other DNA
cggacttacgtagaaaggaaacgatcattgtgtccg

SEQ ID NO: 81 - DNA, 36 bp, synthetic construct
  source 1..36: mol_type = other DNA
cggacttacgtcgaaaggtaacgatcattgtgtccg

SEQ ID NO: 82 - DNA, 36 bp, synthetic construct
  source 1..36: mol_type = other DNA
cggtcttacgtagaaaggtaacgatcattgtggccg

SEQ ID NO: 83 - DNA, 31 bp, synthetic construct
  source 1..31: mol_type = other DNA
cggacttacgtagaaaggtaacgatcattgt

SEQ ID NO: 84 - DNA, 36 bp, synthetic construct
  source 1..36: mol_type = other DNA
cggacttacgtagagaggtaacgatcattgtgtccg

SEQ ID NO: 85 - DNA, 36 bp, synthetic construct
  source 1..36: mol_type = other DNA
cggtcttacgtagaaaggtaacgatcattgtgaccg

SEQ ID NO: 86 - DNA, 36 bp, synthetic construct
  source 1..36: mol_type = other DNA
cggacttacgtagaaaggcaacgatcattgtgtccg

SEQ ID NO: 87 - DNA, 37 bp, synthetic construct
  source 1..37: mol_type = other DNA
cggacttacgtagaaaggtaacgatcatttgtgtccg

SEQ ID NO: 88 - DNA, 35 bp, synthetic construct
  source 1..35: mol_type = other DNA
cggacttcgtagaaaggtaacgatcattgtgtccg

SEQ ID NO: 89 - DNA, 36 bp, synthetic construct
  source 1..36: mol_type = other DNA
cgggcttacgtagaaaggaaacgatcattgtgtccg

SEQ ID NO: 90 - DNA, 37 bp, synthetic construct
  source 1..37: mol_type = other DNA
cggactttacgtagaaaggtaacgatcattgtgtccg

SEQ ID NO: 91 - DNA, 35 bp, synthetic construct
  source 1..35: mol_type = other DNA
cggactacgtagaaaggtaacgatcattgtgtccg

SEQ ID NO: 92-99 - skipped (8 entries)

SEQ ID NO: 100 - RNA, 22 nt, synthetic construct
  source 1..22: mol_type = other RNA
ygtrgaaakgtaamgatcrttg

SEQ ID NO: 101 - RNA, 22 nt, synthetic construct
  source 1..22: mol_type = other RNA
tgtrgaaakgtaamgatcgttg

SEQ ID NO: 102 - RNA, 23 nt, synthetic construct
  source 1..23: mol_type = other RNA
  misc_difference 16: note = a or absent
cccggwgggatagganttatcgg

SEQ ID NO: 103-104 - skipped (2 entries)

SEQ ID NO: 105 - RNA, 22 nt, synthetic construct
  source 1..22: mol_type = other RNA
cgtagaaaggtaacgatcattg

SEQ ID NO: 106-107 - skipped (2 entries)

SEQ ID NO: 108 - RNA, 22 nt, synthetic construct
  source 1..22: mol_type = other RNA
ygtrgaaakgtaamgatcrttg

SEQ ID NO: 109 - skipped

SEQ ID NO: 110 - RNA, 19 nt, synthetic construct
  source 1..19: mol_type = other RNA
cggagactcggtagacttc

SEQ ID NO: 111 - RNA, 22 nt, synthetic construct
  source 1..22: mol_type = other RNA
agtagaaaggtaacgatcattg

SEQ ID NO: 112 - RNA, 15 nt, synthetic construct
  source 1..15: mol_type = other RNA
tgtcgtatcgattcg

SEQ ID NO: 113 - RNA, 19 nt, synthetic construct
  source 1..19: mol_type = other RNA
ggcagagtcggtgggctta

SEQ ID NO: 114 - RNA, 22 nt, synthetic construct
  source 1..22: mol_type = other RNA
cgtagaaaggtaacgatcattg

SEQ ID NO: 115 - RNA, 15 nt, synthetic construct
  source 1..15: mol_type = other RNA
tgctcactgtcatgt

SEQ ID NO: 116 - RNA, 20 nt, synthetic construct
  source 1..20: mol_type = other RNA
tgaaaaattagtcggcttac

SEQ ID NO: 117 - RNA, 21 nt, synthetic construct
  source 1..21: mol_type = other RNA
gtagaaaggtaacgatcattg

SEQ ID NO: 118 - RNA, 15 nt, synthetic construct
  source 1..15: mol_type = other RNA
tgctgactgaagtct

SEQ ID NO: 119 - skipped

SEQ ID NO: 120 - RNA, 22 nt, synthetic construct
  source 1..22: mol_type = other RNA
cgtagaaaggtaacgatcattg

SEQ ID NO: 121-122 - skipped (2 entries)

SEQ ID NO: 123 - RNA, 22 nt, synthetic construct
  source 1..22: mol_type = other RNA
cgtagaaaggtaacgatcattg

SEQ ID NO: 124-125 - skipped (2 entries)

SEQ ID NO: 126 - RNA, 22 nt, synthetic construct
  source 1..22: mol_type = other RNA
cgtagaaaggtaacgatcattg

SEQ ID NO: 127-128 - skipped (2 entries)

SEQ ID NO: 129 - RNA, 22 nt, synthetic construct
  source 1..22: mol_type = other RNA
tgtggaaatgtaaagatcgttg

SEQ ID NO: 130-131 - skipped (2 entries)

SEQ ID NO: 132 - RNA, 22 nt, synthetic construct
  source 1..22: mol_type = other RNA
tgtggaaatgtaaagatcgttg

SEQ ID NO: 133 - RNA, 20 nt, synthetic construct
  source 1..20: mol_type = other RNA
ccgcgaacgacggttgtttt

SEQ ID NO: 134 - skipped

SEQ ID NO: 135 - RNA, 22 nt, synthetic construct
  source 1..22: mol_type = other RNA
tgtggaaatgtaaagatcgttg

SEQ ID NO: 136 - RNA, 22 nt, synthetic construct
  source 1..22: mol_type = other RNA
ccgcgaacgacggttgtttttt

SEQ ID NO: 137 - RNA, 19 nt, synthetic construct
  source 1..19: mol_type = other RNA
gggagagtcggtagcagtc

SEQ ID NO: 138 - RNA, 22 nt, synthetic construct
  source 1..22: mol_type = other RNA
tgtagaaaggtaacgatcgttg

SEQ ID NO: 139 - RNA, 15 nt, synthetic construct
  source 1..15: mol_type = other RNA
gactcactgtaatct

SEQ ID NO: 140 - skipped

SEQ ID NO: 141 - RNA, 21 nt, synthetic construct
  source 1..21: mol_type = other RNA
gtggaaatgtaaagatcgttg

SEQ ID NO: 142 - RNA, 12 nt, synthetic construct
  source 1..12: mol_type = other RNA
ccgcgaacgacg

SEQ ID NO: 143 - RNA, 19 nt, synthetic construct
  source 1..19: mol_type = other RNA
cgtagacgatttaccggtc

SEQ ID NO: 144 - RNA, 22 nt, synthetic construct
  source 1..22: mol_type = other RNA
cgtagaaatgtaaagatcgttg

SEQ ID NO: 145 - RNA, 15 nt, synthetic construct
  source 1..15: mol_type = other RNA
gactcggtgtctact

SEQ ID NO: 146 - skipped

SEQ ID NO: 147 - RNA, 23 nt, synthetic construct
  source 1..23: mol_type = other RNA
cccggagggataggaattatcgg

SEQ ID NO: 148-149 - skipped (2 entries)

SEQ ID NO: 150 - RNA, 22 nt, synthetic construct
  source 1..22: mol_type = other RNA
cccggtgggataggattatcgg

SEQ ID NO: 151 - RNA, 16 nt, synthetic construct
  source 1..16: mol_type = other RNA
ttgcataagccattgt

SEQ ID NO: 152 - skipped

SEQ ID NO: 153 - RNA, 22 nt, synthetic construct
  source 1..22: mol_type = other RNA
cccggagggataggattatcgg

SEQ ID NO: 154 - RNA, 17 nt, synthetic construct
  source 1..17: mol_type = other RNA
tcttgagatgcaaactt

SEQ ID NO: 155 - RNA, 10 nt, synthetic construct
  source 1..10: mol_type = other RNA
tcccatagat

SEQ ID NO: 156 - RNA, 23 nt, synthetic construct
  source 1..23: mol_type = other RNA
cccggagggataggaattatcgg

SEQ ID NO: 157 - RNA, 14 nt, synthetic construct
  source 1..14: mol_type = other RNA
atctaaacgtctgt

SEQ ID NO: 158 - RNA, 21 nt, synthetic construct
  source 1..21: mol_type = other RNA
ttgggcgtctcgccattatga

SEQ ID NO: 159 - RNA, 22 nt, synthetic construct
  source 1..22: mol_type = other RNA
cccggtgggataggattatcgg

SEQ ID NO: 160-161 - skipped (2 entries)

SEQ ID NO: 162 - RNA, 21 nt, synthetic construct
  source 1..21: mol_type = other RNA
cccggtgggataggattatcg

SEQ ID NO: 163-164 - skipped (2 entries)

SEQ ID NO: 165 - RNA, 22 nt, synthetic construct
  source 1..22: mol_type = other RNA
cccggagggataggattatcgg

SEQ ID NO: 166 - skipped

SEQ ID NO: 167 - DNA, 81 bp, synthetic construct
  source 1..81: mol_type = other DNA
gggagattccgcagctagtgttcggacttacgtagaaaggtaacgatcattgtgtccgtgcctcttcctaacggtaaccaa

SEQ ID NO: 168 - DNA, 22 bp, synthetic construct
  source 1..22: mol_type = other DNA
agggagattccgcagctagtgt

SEQ ID NO: 169 - DNA, 22 bp, synthetic construct
  source 1..22: mol_type = other DNA
ttggttaccgttaggaagaggc

SEQ ID NO: 170 - RNA, 40 nt, synthetic construct; identical to SEQ ID NO: 72
  source 1..40: mol_type = other RNA

SEQ ID NO: 171 - RNA, 48 nt, synthetic construct; identical to SEQ ID NO: 55
  source 1..48: mol_type = other RNA
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-fluoro modified nucleotide
  modified_base 9: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 16: mod_base = OTHER; note = 2'-fluoro modified nucleotide
  modified_base 19: mod_base = OTHER; note = 2'-fluoro modified nucleotide
  modified_base 20: mod_base = OTHER; note = 2'-fluoro modified nucleotide
  modified_base 23: mod_base = OTHER; note = 2'-fluoro modified nucleotide
  modified_base 26: mod_base = OTHER; note = 2'-fluoro modified nucleotide
  modified_base 27: mod_base = OTHER; note = 2'-fluoro modified nucleotide
  modified_base 30: mod_base = OTHER; note = 2'-fluoro modified nucleotide
  modified_base 31: mod_base = OTHER; note = 2'-fluoro modified nucleotide
  modified_base 32: mod_base = OTHER; note = 2'-fluoro modified nucleotide
  modified_base 34: mod_base = OTHER; note = 2'-fluoro modified nucleotide
  modified_base 37: mod_base = OTHER; note = 2'-fluoro modified nucleotide
  modified_base 38: mod_base = OTHER; note = 2'-fluoro modified nucleotide
  modified_base 41: mod_base = OTHER; note = 2'-fluoro modified nucleotide
  modified_base 44: mod_base = OTHER; note = 2'-fluoro modified nucleotide
  modified_base 45: mod_base = OTHER; note = 2'-fluoro modified nucleotide
  modified_base 46: mod_base = OTHER; note = 2'-fluoro modified nucleotide
  modified_base 47: mod_base = OTHER; note = 2'-fluoro modified nucleotide
  modified_base 48: mod_base = OTHER; note = 2'-fluoro modified nucleotide

SEQ ID NO: 172 - DNA, 36 bp, synthetic construct
  source 1..36: mol_type = other DNA
ggcgtagtgattatgaatcgtgtgctaatacacgcc